(19) United States
(12) Patent Application Publication
(10) Pub. No.: US 2014/0295448 A1
(43) Pub. Date: Oct. 2, 2014

(54) SUPPRESSION OF TLA2-CpFTSY GENE EXPRESSION FOR IMPROVED SOLAR ENERGY CONVERSION EFFICIENCY AND PHOTOSYNTHETIC PRODUCTIVITY IN ALGAE

(71) Applicant: THE REGENTS OF THE UNIVERSITY OF CALIFORNIA, Oakland, CA (US)

(72) Inventors: Anastasios Melis, El Cerrito, CA (US); Henning Kirst, Burgwedel (DE)

(73) Assignee: THE REGENTS OF THE UNIVERSITY OF CALIFORNIA, Oakland, CA (US)

(21) Appl. No.: 14/353,593

(22) PCT Filed: Oct. 24, 2012
(86) PCT No.: PCT/US2012/061555

§ 371 Date: Apr. 23, 2014

Related U.S. Application Data
(60) Provisional application No. 61/550,872, filed on Oct. 24, 2011.

(51) Int. Cl. C12N 15/113; C12P 5/00; C12P 7/02; C12Q 1/68; C12P 7/64

(52) U.S. Cl. CPC C12N 15/113; C12Q 1/686; C12P 7/649; C12P 7/02; C12P 5/002; C12P 5/007
USPC 435/6.12; 435/134; 435/155; 435/166; 435/167; 435/257.1; 435/257.3; 435/257.5; 435/257.6; 204/456

(57) ABSTRACT

The invention provides method and compositions to minimize the chlorophyll antenna size of photosynthesis by decreasing TLA2 gene expression, thereby improving solar conversion efficiencies and photosynthetic productivity in green microalgae, under bright sunlight conditions.

### CROSS-REFERENCE TO RELATED APPLICATIONS

This application claims priority benefit of U.S. provisional application No. 61/550,872. filed Oct. 24, 2011, which application is herein incorporated by reference.

### STATEMENT AS TO RIGHTS TO INVENTIONS MADE UNDER FEDERALLY SPONSORED RESEARCH AND DEVELOPMENT

This invention was made with Government support under Grant No. DE-FG36-05GO15041 awarded by the Department of Energy. The Government has certain rights in this invention.

### BACKGROUND OF THE INVENTION

Photosynthesis depends on the absorption of sunlight by chlorophyll (Chl) molecules in photosystem-I (PSI) and photosystem-II (PSII). In higher plants and green algae, a completely functional but minimal PSI unit encompasses 95 Chl a molecules, while PSII functions with a minimal number of 37 Chl a molecules (Glick & Melis. Biochim Biophys Acta 934: 151-155, 1988; Jordan et al., Nature 411(6840): 909-917, 2001: Zouni et al., Nature 409: 739-743, 2001; Ruban et al., Nature 421: 648-652, 2003). Increasing the number of light-harvesting pigments associated with each reaction center, upon the addition of peripheral chlorophyll a and b molecules, is thought to afford a competitive advantage to the organism in an environment where sunlight is often limiting (Kirk, Light and photosynthesis in aquatic ecosystems, 2nd edn. Cambridge University Press, Cambridge, England. 1994). Photosynthetic organisms evolved a variety of strategies and pigment-containing protein complexes associated peripherally with PSI and PSII. In higher plants and algae, these are referred to as Chl a-b LHC-I and LHC-II for PSI and PSII, respectively. Photosystem-peripheral LHCs serve as auxiliary antennae for the collection of sunlight energy and as a conducting medium for excitation energy migration towards a photochemical reaction center (Smith et al., 1990). The Chl a-b LHCs increase the number of pigment molecules that are associated with the reaction centers, normally up to 250 for PSI and 300 for PSII (Ley and Mauzerall, 1982; Melis and Anderson. 1983; Smith et al. 1990. Melis, 1991).

The Chl antenna size of the photosystems is not fixed but is regulated by the level of irradiance seen by the photosynthetic apparatus (Smith et al., 1990; Melis, 1991; Ballottari et al., 2007). However. genes that direct a large size for the Chl antenna, and those that regulate the assembly of the LHCs are not well understood. In the green algae three genes are known to influence the accumulation of light-harvesting complexes in the thylakoid membrane, namely ALB3.1, TLA1 and NAB1 (Bellafiore et al., 2002; Polle et al., 2003; Mussgnug et al., 2005; Tetali et al, 2007; Mitra and Melis, 2010). The nucleic acid binding protein NAB1 binds to the mRNA of the major Lhcb genes and thereby represses their translation (Mussgnug et al., 2005). Consequently, a deletion of the NAB1 gene de-represses Lhcb translation, leading to a larger Chl antenna size phenotype in NAB1-minus mutants. Plants exhibiting suppression of the TLA1 gene have a truncated light-harvesting Chl antenna size for both photosystems (e.g., U.S. Pat. No. 7,745,696). The highly conserved among eukaryotes TLA1 protein was postulated to help define the relationship between nucleus and organelle by an as yet unknown mechanism (Tetali et al., 2007; Mitra and Melis, 2010). ALB3, the product of the AlB3.1 gene, is a homologue of YidC of E. coli, which is an inner membrane protein that facilitates incorporation of transmembrane proteins by the so-called signal recognition particle (SRP) (Yi and Dalbey, 2005). In Chlamydomonas reinhardtii, ALB3 is nuclear encoded but targeted to the chloroplast. It is important for the incorporation of the peripheral light-harvesting complexes into the thylakoid membrane of photosynthesis (Bellafiore et al., 2002). The ALB3 protein is also known in Arabidopsis thaliana but its function appears to extend beyond the transmembrane integration of light-harvesting complexes, as it appears to also be needed for the assembly of PSI and PSII in the thylakoid membrane (Asakura et al., 2008).

The chloroplast signal recognition particle (SPR) is defined as a collection of four proteins that work together, including CpSRP54, CpSRP43, CpFTSY and ABL3 (recent review: Aldridge et al., 2009). It is postulated that CpSRP54 and CpSRP43 operate in the chloroplast stroma, where they bind to proteins targeted for insertion in the thylakoid membrane. The receptor CpFTSY protein recognizes the CpSRP54-CpSRP43-target protein complex and guides the complex to the integral thylakoid membrane protein ABL3. The latter facilitates the incorporation of the target protein into the thylakoid membrane. A Zea mays null cpftsy mutation caused the loss of various LHC complexes and the thylakoid-bound photosynthetic enzyme complexes and was seedling lethal (Asakura et al. 2004). An Arabidopsis thaliana knockout mutant of CpFTSY was missing most of the light harvesting proteins, but was also deficient in PSI and PSTI core proteins from the thylakoid membrane (Asakura et al., 2008). The cpftsy mutant of Arabidopsis was also seedling lethal. A similar conclusion was reached for the alb3 mutant of Arabidopsis (Asakura et al., 2008). The CpSRP component proteins in higher plants, namely CpSRP54 and CpSRP43, are postulated to be involved in the proper folding of light-harvesting proteins and targeting to the thylakoid membrane, thereby facilitating the biogenesis and assembly of the photosystem holocomplexes (Pilgrim et al., 1998; Klimyuk et al. 1999).

### BRIEF SUMMARY OF THE INVENTION

The current invention is based, in part, on the discovery that suppression of the gene Tla2 results in reduced chlorophyll antenna size in green microalgae. Thus, in one aspect, the invention relates to a method of decreasing chlorophyll antenna size in a green microalgae, the method comprising: inhibiting expression of a Tla2 nucleic acid in the green microalgae by introducing into the plant an expression cassette comprising a promoter operably linked to a polynucleotide, or a complement thereof, that specifically hybridizes to a nucleic acid encoding SEQ ID NO:2 or to a nucleic acid that has at least 70% identity, often at least 80%, 90%, or 95% identity, to at least 200 contiguous, or at least 500 contiguous nucleotides or at least 1,000 contiguous nucleotides of a sequence encoding SEQ ID NO:2; and selecting a green microalgae with decreased chlorophyll antenna size compared to a green microalgae in which the expression cassette has not been introduced. The promoter may be inducible or constitutive. In some embodiments the polynucleotide is operably linked to the promoter in the antisense orientation; in other embodiments, the polynucleotide is operably linked to the promoter in the sense orientation.

In some embodiment, the polynucleotide introduced into a green microalgae, is an siRNA. In other embodiments, the polynucleotide is an antisense RNA.

The nucleic acid to which the polynucleotide hybridizes can encode a polypeptide of SEQ ID NO:2. In particular embodiments, the nucleic acid is SEQ ID NO:1 or SEQ ID NO:3.

In some embodiments the green microalgae into which the nucleic acid is introduced is selected from Chlamydomonas reinhardtii, Scenedesmus obliquus, Chlorella, Nannochloropsis, Botryococcus, including Botryococcus braunii and Botryococcus sudeticus, Dunaliella salina, or Haematococcus pluvialis.

In another aspect, the invention also relates to a green microalgae that contains an expression cassette comprising a heterologous polynucleotide, or a complement thereof, that specifically hybridizes to a nucleic acid that encodes SEQ ID NO:2 or to a nucleic acid that has at least 70% percent identity, often at least 80%, 90%, or 95% identity, to at least 200 contiguous nucleotides, or at least 500 contiguous nucleotides or at least 1,000 contiguous nucleotides of a sequence encoding SEQ ID NO:2. In some embodiments, the plant is a green microalgae selected from Chlamydomonas reinhardtii, Scenedesmus obliquus, Chlorella, Nannochloropsis, Botryococcus, including Botryococcus braunii and Botryococcus sudeticus, Dunaliella salina, or Haematococcus pluvialis.

The invention additionally relates to a method of enhancing yields of photosynthetic productivity under high-density growth conditions, the method comprising cultivating a Tla2-suppressed green algae of the invention, such as Chlamydomonas reinhardtii, Scenedesmus obliquus, Chlorella, Nannochloropsis, Botryococcus, including Botryococcus braunii and Botryococcus sudeticus, Dunaliella salina, or Haematococcus pluvialis under bright sunlight and high density growth conditions.

Additionally, the invention relates to a method of enhancing H₂ production, the method comprising suppressing Tla2 gene expression in a green microalgae, e.g., Chlamydomonas reinhardtii, Scenedesmus obliquus, Chlorella sp. to be used for H₂ production; and cultivating the algae under conditions in which H₂ is produced.

The invention further relates to a method of enhancing bio-oil or bio-diesel production, the method comprising suppressing Tla2 gene expression in a green microalgae, e.g. Chlorella, Nannochloropsis, and Botryococcus sp. such as Botryococcus braunii or Botryococcus sudeticus, to be used for bio-oil or bio-diesel production; and cultivating the algae under conditions in which bio-oil or bio-diesel is produced.

Further, the invention relates to a method of enhancing beta-carotene, lutein or zeaxanthin production, the method comprising suppressing Tla2 gene expression in a green microalgae, e.g., Dunaliella salina, to be used for beta-carotene. lutein or zeaxanthin production; and cultivating the algae under conditions in which beta-carotene, lutein or zeaxanthin is produced.

In other embodiments, the invention relates to a method of enhancing astaxanthin production, the method comprising suppressing Tla2 gene expression in a green microalgae, e.g., Haematococcus pluvialis, to be used for astaxanthin production; and cultivating the algae under conditions in which astaxanthin is produced.

In another aspect, the invention relates to a method of screening for green microalgae that show enhanced yield of photosynthetic productivity, the method comprising: introducing a mutation into a population of green microalgae; and screening for inhibition of Tla2 gene expression, wherein inhibition of Tla2 gene expression is determined by measuring the level of Tla2 mRNA or Tla2 protein. In some embodiments, the green microalgae are selected from Chlamydomonas reinhardtii, Scenedesmus obliquus, Chlorella, Nannochloropsis, Botryococcus, including Botryococcus braunii and Botryococcus sudeticus, Dunaliella salina, or Haematococcus pluvialis.

### BRIEF DESCRIPTION OF THE DRAWINGS

FIG. 1: Single-cell colonies of Chlamydomonas reinhardtii wild type and tla2 mutant grown on agar. The wild type strains have a darker coloration, as compared to the light coloration of the tla2 mutant.

FIG. 2: Light-saturation curves of photosynthesis obtained with the C. reinhardtii wild type (solid squares) and the tla2 mutant (open circles). The initial slopes of both curves are similar, indicating equal quantum yield of the photosynthesis. The light-saturated rate Pₘₐₓ was greater in the tla2 mutant than in the wild type, indicating a greater productivity on a per Chl basis in the tla2 than in the wild type.

FIG. 3: Southern blot analysis to define copy number and integrity of inserted pJD67 plasmid into the genomic DNA of Chlamydomonas reinhardtii insertional transformant. Wild type, tla2, and tla3 (an independent truncated antenna mutant) strains were used in this analysis. A Schematic map of pJD56. Dark gray boxes indicate the promoter and terminator region of the ARG7 gene. These regions and the ARG7 gene are not plasmid specific but are also present in the host strain. Isolated genomic DNA was digested by BanI, PstI, NcoI and SmaI. The location of probes 1-6, used for the Southern blot analysis, are marked with black lines. B Hybridized Southern blot results. Each column indicates the probe used for hybridization and consist of a set of four genomic DNA digests, BanI and PstI in the upper row, NcoI and SmaI in the lower row. The digested genomic DNA was loaded as follows: 1: tla2; 2: CC425; 3: positive control (tla3). Marker sizes indicate electrophoretic mobility in kb.

FIG. 4: DNA insertional mutagenesis-induced reorganization of the genomic DNA in the tla2 strain. A. Schematic comparing the genomic DNA on chromosome 5 in wild type (upper map) and tla2 mutant (lower map). Arrows mark genes and their orientation. Dashed lines indicate the rearrangement of the genomic DNA in the tla2 mutant. Genes that are deleted in the tla2 mutant are indicated. Probes used for hybridization are marked with bars: white bar—5′ probe, hatched bar—3′ probe and black bar—deletion probe. The expected size of fragments generated upon digestion with FspI and SacI are marked in kb. B. Southern hybridized blots. Probes used for hybridization are indicated on top of each panel. Restriction enzymes used for digestion and the genomic DNA sample are indicated in the bottom of the blot. The marker on the left indicates the electrophoretic mobility in kb.

FIG. 5: Genetic cross analysis of tla2 with AG1x3.24 (arg2) strain. One representative tetrad from a single cross is shown, plated on non-selective TAP+ARG media (top panel) or selective TAP-only media (middle panel). The Chl a/Chl b ratio of these progeny is shown at the top of the panels. The lower panel shows the result of PCR reactions, two lanes per progeny: the PCR reaction using an insertion specific primer-set was loaded on lanes 1, 3, 5, 7, and a positive control PCR on lanes 2, 4, 6, 8.

FIG. 6: (Upper) Amino acid sequence of the Chlamydomonas reinhardtii FTSY protein. Domains of the CrCpFtsY protein are as follows: Amino acids 1-36: Transit peptide; Amino acids 66-147: Helical bundle domain (Pfam), SRP54-type-protein; Amino acids 162-370: GTPase domain (Pfam), SRP54-type protein; Amino acids 164-183: P-loop nucleotide binding motif, (pre); Amino acids 170-176, 258-262 & 322-325: Homolgous nucleotide binding: (Lower) Domain presentation of the CrCpFTSY protein. CpTP: chloroplast transit peptide. HB: Helical bundle domain. GTPase: GTPas domain.

FIG. 7: Western blot analysis of the light-harvesting antenna proteins of PSII in wild type and the tla2 mutant. A. Immuno-detection of proteins with specific polyclonal antibodies against the light harvesting proteins Lhcb1/Ihcb2. Lhcb3. Lhcb4 and Lhcb5, the PSII reaction center protein D2, the PSI reaction center protein PsaL, RuBisCo and the β subunit of the ATP synthase are shown. B. Coomassie-blue stained SDS-PAGE analysis of the samples shown in A.

FIG. 8: Western blot analysis of C. reinhardtii total cell protein extracts isolated from wild type, the tla2 mutant strain, and tla2 lines C1, C2, C3, C4 complemented with a wild type copy of the CrCpFTSY gene. A. Immuno-detection of CrCpFTSY and specific thylakoid membrane proteins was performed with polyclonal antibodies against PSII subunits CP43 and PsbO, Photosystem I subunit PsaL and LHCII subunit Lhcb1. Loading of lanes was based on Chl and corrected for the Chl content per cell so as to load proteins on an equal cells basis. B. Coomassie-blue stained SDS-PAGE analysis of the samples shown in A.

FIG. 9: Cell fractionation and localization of the CpFTSY protein. A Immunoblot analysis of wild-type total cell protein extract (1.5 μg Chl loaded), total membrane extract (1.5 μg Chl loaded), total soluble fraction (75 μg of protein loaded), and isolated chloroplast extract (1.5 μg Chl loaded). Western blot analysis was conducted with specific polyclonal antibodies raised against the CrCpFTSY, CrCpSRP54, PsbO or D2 proteins. B Coomassie-blue stained SDS-PAGE analysis of the samples shown in A.

FIG. 10: Analysis of photosynthetic complexes from thylakoid membranes, resolved by non-denaturing deriphat PAGE and denaturing second dimension electrophoresis. Samples tested were from wild type, tla2 mutant, and tla2 lines C1, C2, C3, C4 complemented with a wild type copy of the CrCpFTSY gene. A. Pigment-protein complexes resolved by non-denaturing deriphat-PAGE. Protein complexes were identified by their molecular mass of the first non-denaturing and second denaturing dimension. Masses of the marker on the left are given in kD. B Silver nitrate stained second denaturing dimension from wild type and tla2. 1: PSI reaction center proteins PsaA and PsaB dimer, 2: LHCI proteins, 3: PSII reaction center proteins CP43 and CP47, 4: PSII reaction center proteins D1 and D2, 5: LCHII proteins, 6: α and β subunit of the ATP-synthase. Molecular size markers are given in kD.

FIG. 11: Example of a working model of the function of the CrCpSRP transmembrane complex assembly system in the model green algae C. reinhardtii. Precursor light-harvesting proteins (LHC-protein) are targeted to the chloroplast via the transit peptide and the heat shock protein HSP70, which functions as a molecular chaperon to prevent aggregation of the pre-assembled proteins. Chloroplast protein import is facilitated by the envelope-localized TOC and TIC complexes, which catalyze protein import through the outer and inner envelope membranes of the chloroplast. The transit peptide is cleaved off and the molecular chaperon CpSRP43 binds to the incoming light-harvesting protein to prevent its aberrant misfolding. CpSRP54 and CpFTSY guide this CpSRP43-LHC complex to the membrane-bound translocase ALB3. Upon integration of the light-harvesting protein into the nascent thylakoid membrane, the LHC-CPSRP43-CPSRP54-CpFTSY complex disassembles, making the SRP subunits available for another carry-and-assembly cycle.

FIG. 12: Phylogenetic relationships of the TLA2-CpFTSY protein in photosynthetic eukaryotes. The maximum likelihood method was used to construct the phylogenetic tree displaying the evolutionary relationship of the TLA2-CpFTSY protein in photosynthetic eukaryotes based on their amino acid sequences. Evolutionary distance is given in the form of “changes per position”.

### DETAILED DESCRIPTION OF THE INVENTION

### Definitions

The terms “Tla2” and “CpFTSY” are used interchangeably to refer to a polynucleotides and polypeptides that are encoded by a locus that encodes for one of the components of the chloroplast Signal Recognition Particle (SRP), namely the nuclear-encoded and chloroplast-localized FTSY protein.

In the context of the present invention, a “Tla2 polynucleotide” is a nucleic acid sequence substantially similar to SEQ ID NO:1 or SEQ ID NO:3, or that encodes a polypeptide that is substantially similar to SEQ ID NO:2. Tla2 polynucleotides may comprise (or consist of) a region of about 15 to about 1,000 or more nucleotides, sometimes from about 20, or about 50, to about 1,100 nucleotides and sometimes from about 200 to about 600 nucleotides, which hybridizes to SEQ ID NO:1 or SEQ ID NO:3, or the complements thereof, under stringent conditions, or which encodes a Tla2 polypeptide or fragment of at least 15 amino acids thereof. Tla2 polynucleotides can also be identified by their ability to hybridize under low stringency conditions (e.g., Tm ˜40° C.) to nucleic acid probes having the sequence of SEQ ID NO:1 or SEQ ID NO:3. Such Tla2 nucleic acid sequence can have, e.g., about 25-30% base pair mismatches or less relative to the selected nucleic acid probe. SEQ ID NOs: 1 and 3 are examples of Tla2 polynucleotide sequences. The term “Tla2 polynucleotide” encompasses antisense as well as sense nucleic acids.

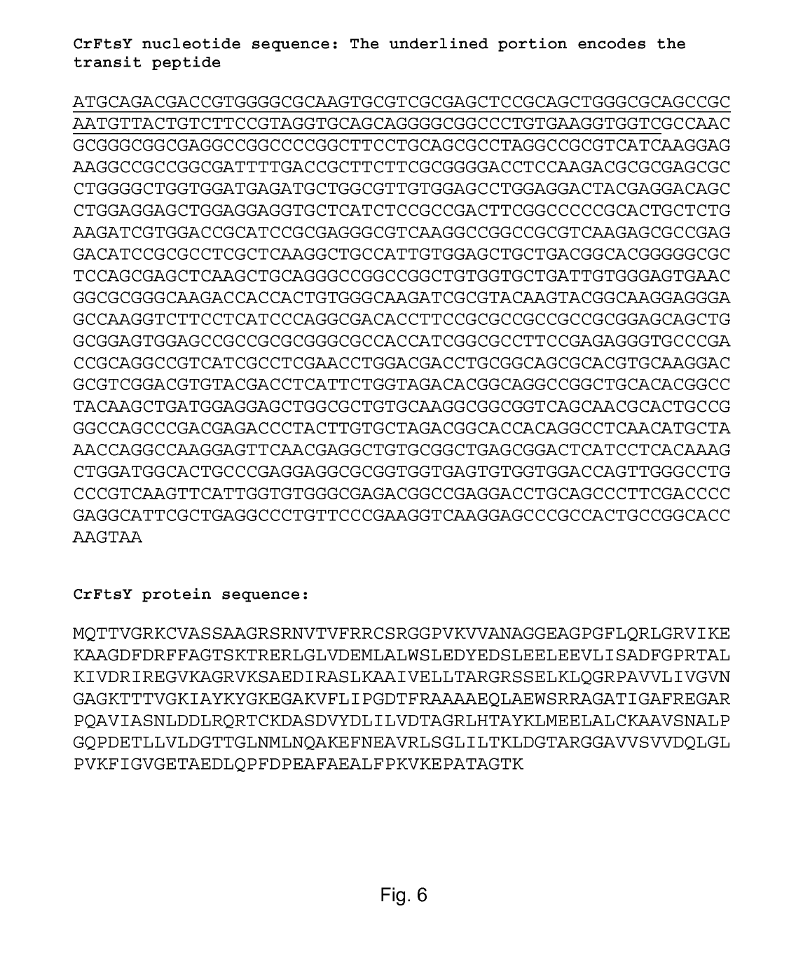

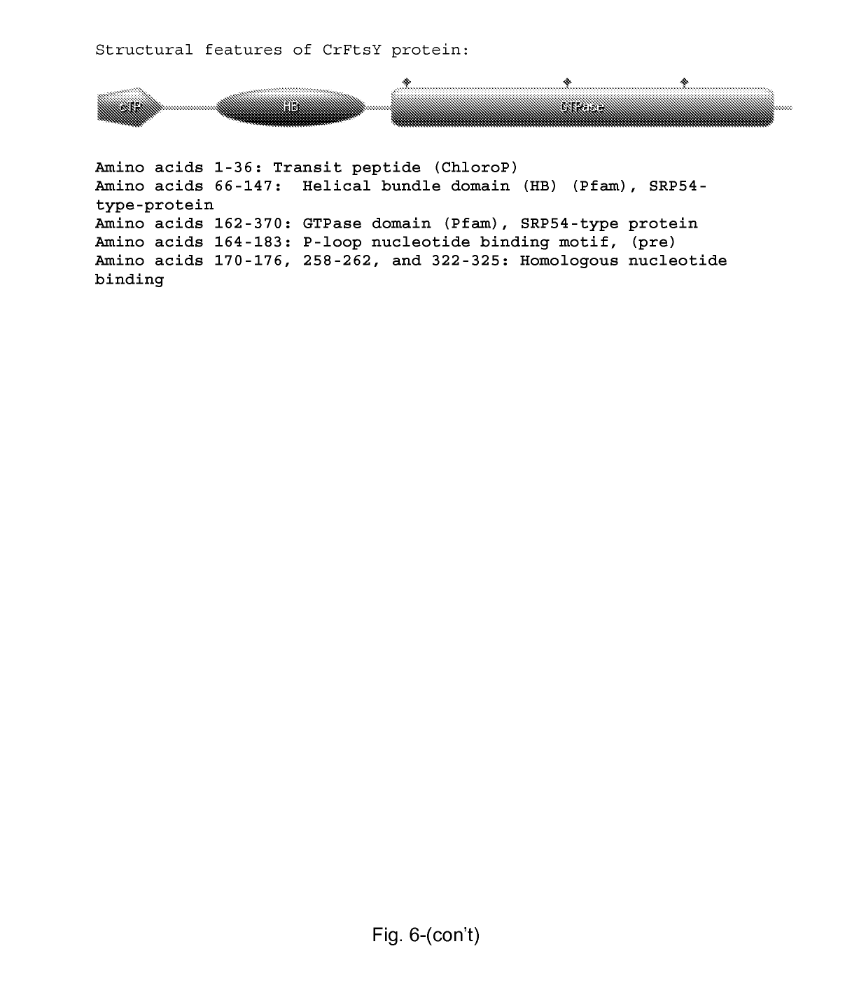

A “Tla2 polypeptide” is an amino acid sequence that is substantially similar to SEQ ID NO:2. or a fragment or domain thereof. A full-length Tla2 protein from the green microalgae Chlamydomonas reinhardtii is 381 amino acids. The domain structure of Tla2 protein based on SEQ ID NO:2 is shown in FIG. 6. The domains are highly conserved in green microalgae.

As used herein, a homolog or ortholog of a particular Tla2 gene (e.g., SEQ ID NO:3) is a second gene in the same green microalgae species or in a different species which has a polynucleotide sequence of at least 50 contiguous nucleotides, typically at least 100, 500, 1000, or 2,000 or more contiguous nucleotides that are substantially identical (determined as described below) to a sequence in the first gene.

The terms “nucleic acid” and “polynucleotide” are used synonymously and refer to a single or double-stranded polymer of deoxyribonucleotide or ribonucleotide bases read from 10 the 5′ to the 3′ end. A nucleic acid of the present invention will generally contain phosphodiester bonds, although in some cases, nucleic acid analogs may be used that may have alternate backbones, comprising, e.g., phosphoramidate, phosphorothioate, phosphorodithioate, or O-methylphosphoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, and non-ribose backbones. Thus, nucleic acids or polynucleotides may also include modified nucleotides, that permit correct read through by a polymerase. “Polynucleotide sequence” or “nucleic acid sequence” may include both the sense and antisense strands of a nucleic acid as either individual single strands or in a duplex. As will be appreciated by those in the art, the depiction of a single strand also defines the sequence of the complementary strand; thus the sequences described herein also provide the complement of the sequence. Unless otherwise indicated, a particular nucleic acid sequence also implicitly encompasses conservatively modified variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc

The phrase “nucleic acid sequence encoding” refers to a nucleic acid that codes for an amino acid sequence of at least 5 contiguous amino acids within one reading frame. The amino acid need not necessarily be expressed when introduced into a cell or other expression system, but may merely be determinable based on the genetic code. Thus, a polynucleotide may encode a polypeptide sequence that comprises a stop codon or contains a changed frame so long as at least 5 contiguous amino acids within one reading frame. The nucleic acid sequences may include both the DNA strand sequence that is transcribed into RNA and the RNA sequence. The nucleic acid sequences include both the full-length nucleic acid sequences as well as fragments from the full-length sequences. It should be further understood that the sequence includes the degenerate codons of the native sequence or sequences, which may be introduced to provide codon preference in a specific host cell.

The term “promoter” or “regulatory element” refers to a region or sequence determinants located upstream or downstream from the start of transcription that are involved in recognition and binding of RNA polymerase and other proteins to initiate transcription. A “plant promoter” is a promoter capable of initiating transcription in plant cells. Such promoters need not be of plant origin, for example, promoters derived from plant viruses, such as the CaMV35S promoter, can be used in the present invention.

As used herein, the term “algal regulatory element” or “algae promoter” refers to a nucleotide sequence that, when operatively linked to a nucleic acid molecule, confers e expression upon the operatively linked nucleic acid molecule in unicellular green algae, which are also referred to herein as “green microalgae”. It is understood that limited modifications can be made without destroying the biological function of a regulatory element and that such limited modifications can result in algal regulatory elements that have substantially equivalent or enhanced function as compared to a wild type algal regulatory element. These modifications can be deliberate, as through site-directed mutagenesis, or can be accidental such as through mutation in hosts harboring the regulatory element. All such modified nucleotide sequences are included in the definition of an algal regulatory element as long as the ability to confer expression in unicellular green algae is substantially retained.

The term “suppressed” or “decreased” encompasses the absence of Tla2 protein in a green microalgae as well as protein expression that is present but reduced in amount as compared to the level of Tla2 protein expression in a wild type green microalgae. The term “suppressed” also encompasses an amount of Tla2 protein that is equivalent to wild type levels, but where the protein has a reduced level of activity in comparison to wild type green microalgae. Generally, at least a 20% decrease in Tla2 activity, amount, chlorophyll antenna size or the like is preferred, with at least about 50% or at least about 75% being particularly preferred.

A polynucleotide sequence is “heterologous to” a second polynucleotide sequence if it originates from a foreign species, or, if from the same species, is modified by human action from its original form. For example, a promoter operably linked to a heterologous coding sequence refers to a coding sequence from a species different from that from which the promoter was derived, or, if from the same species, a coding sequence which is different from any naturally occurring allelic variants.

An “expression cassette” refers to a nucleic acid construct, which when introduced into a host cell, results in transcription and/or translation of a RNA or polypeptide, respectively. Antisense constructs or sense constructs that are not or cannot be translated are expressly included by this definition.

In the case of both expression of transgenes and inhibition of endogenous genes (e.g., by antisense, or sense suppression) one of skill will recognize that the inserted polynucleotide sequence need not be identical and may be “substantially identical” to a sequence of the gene from which it was derived. As explained below, these variants are specifically covered by this term.

In the case where the inserted polynucleotide sequence is transcribed and translated to produce a functional polypeptide, one of skill will recognize that because of codon degeneracy a number of polynucleotide sequences will encode the same polypeptide. These variants are specifically covered by the term “polynucleotide sequence from” a Tla2 gene. In addition, the term specifically includes sequences (e.g., full length sequences) substantially identical (determined as described below) with a Tla2 gene sequence. A “polynucleotide sequence from” a Tla2 gene can encode a protein that retains the function of a Tla2 polypeptide in contributing to chlorophyll antenna size.

In the case of polynucleotides used to inhibit expression of an endogenous gene, the introduced sequence need not be perfectly identical to a sequence of the target endogenous gene. The introduced polynucleotide sequence will typically be at least substantially identical (as determined below) to the target endogenous sequence. Thus, an introduced “polynucleotide sequence from” a Tla2 gene may not be identical to the target Tla2 gene to be suppressed, but is functional in that it is capable of inhibiting expression of the target Tla2 gene.

Two nucleic acid sequences or polypeptides are said to be “identical” if the sequence of nucleotides or amino acid residues, respectively, in the two sequences is the same when aligned for maximum correspondence as described below. The term “complementary to” is used herein to mean that the sequence is complementary to all or a portion of a reference polynucleotide sequence.

Optimal alignment of sequences for comparison may be conducted by the local homology algorithm of Smith and Waterman Add. APL. Math. 2:482 (1981). by the homology alignment algorithm of Needle man and Wunsch J. Mol. Biol. 48:443 (1970), by the search for similarity method of Pearson and Lipman Proc. Natl. Acad. Sci. (U.S.A.) 85: 2444 (1988), by computerized implementations of these algorithms (GAP, BESTFIT, BLAST, FASTA. and TFASTA in the Wisconsin Genetics Software Package. Genetics Computer Group (GCG), 575 Science Dr., Madison, Wis.), or by inspection.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” in the context of polynucleotide sequences means that a polynucleotide comprises a sequence that has at least 50% sequence identity. Alternatively, percent identity can be any integer from 40% to 100%. Exemplary embodiments include at least: 50%, 55%, 60%, 65%, 70%, 75%, 80%, 85%, 90%, 95%, or 99% compared to a reference sequence using the programs described herein; preferably BLAST using standard parameters, as described below. Accordingly, Tla2 sequences of the invention include nucleic acid sequences that have substantial identity, e.g., at least 50%, 55%, 60%, 65%, 70%, 75%. 80%, 85%, 90%, 95%, or 99% identity to SEQ ID NO: 1 or to SEQ ID NO:3 or to the coding region of SEQ ID NO:3.

Another indication that nucleotide sequences are substantially identical is if two molecules hybridize to each other, or a third nucleic acid, under stringent conditions. Stringent conditions are sequence dependent and will be different in different circumstances. Generally, stringent conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Typically, stringent conditions will be those in which the salt concentration is about 0.02 molar at pH 7 and the temperature is at least about 60° C.

In the present invention, mRNA encoded by Tla2 genes of the invention can be identified in Northern blots under stringent conditions using cDNAs of the invention or fragments of at least about 100 nucleotides. For the purposes of this disclosure, stringent conditions for such RNA-DNA hybridizations are those which include at least one wash in 0.2×SSC at 63° C. for 20 minutes, or equivalent conditions. Genomic DNA or cDNA comprising genes of the invention can be identified using the same cDNAs (or fragments of at least about 100 nucleotides) under stringent conditions, which for purposes of this disclosure, include at least one wash (usually 2) in 0.2×SSC at a temperature of at least about 50° C., usually about 55° C., for 20 minutes, or equivalent conditions.

A Tla2 gene for use in the invention can also be amplified using PCR techniques. For example, a Tla2 gene of the invention may be amplifiable using primers shown in the EXAMPLES section.

Tla2 polypeptide sequences of the invention include polypeptide sequences having substantial identify to SEQ ID NO:2. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 50%. Preferred percent identity of polypeptides can be any integer from 50% to 100%, e.g., at least 50%, 55%. 60%, 65%, 70%, 75%, 80%, 85%, 90%, 95%, or 99%, an sometimes at least 61%, 62%, 63%. 64%, 65%, 66%, 67%, 68%, 69%, 70%, 71%, 72%, 73%, 74% and 75%. In typical embodiments, a Tla2 polypeptide sequence has at least 70%, 75%, 80%, 85%, 90%, 95%, or 99%, identity to SEQ ID NO:2. Polypeptides that are “substantially similar” share sequences as noted above except that residue positions that are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, aspartic acid-glutamic acid, and asparagine-glutamine.

The term “isolated”, when applied to a nucleic acid or protein, denotes that the nucleic acid or protein is essentially free of other cellular components with which it is associated in the natural state. It is preferably in a homogeneous state and may be in either a dry or aqueous solution. Purity and homogeneity are typically determined using analytical chemistry techniques such as polyacrylamide gel electrophoresis or high performance liquid chromatography. A protein which is the predominant species present in a preparation is substantially purified. In particular, an isolated gene is separated from open reading frames which flank the gene and encode a protein other than the gene of interest.

### Introduction

The present invention relates to methods of generating green microalgae in which Tla2 gene expression is suppressed and uses of such green microalgae for various purposes. Plants having suppressed Tla2 gene expression exhibit decreases in the size of chlorophyll antenna. Surprisingly, contrary to the observation of seedling-lethal cpftsy mutants in higher plants, tla2 suppressed green microalgae grow well photoautotrophically with a quantum yield of photosynthesis similar to that of the wild type. Thus, green microalgal strains in which Tla2 is suppressed are useful for many purposes, e.g., for mass culture for production of various nutrients or pharmaceuticals, for production of H₂, for production of lipid/hydrocarbons, for carbon sequestration. for wastewater treatment and aquatic pollution amelioration, for atmospheric pollution amelioration, for biomass generation, and for other purposes.

A Tla2 nucleic acid that is targeted for suppression in this invention encodes a Tla2 protein that is substantially identical to SEQ ID NO:2, or a fragment thereof. For example, such Tla2 proteins have one or more conserved domains, designated with reference to SEQ ID NO:2. These domains include the Transit peptide (ChloroP) (amino acids 1-36 with reference to SEQ ID NO:2; the Helical bundle domain (Pfam), SRP54-type-protein (amino acids 66-147 with reference to SEQ ID NO:2; a GTPase domain (Pfam), SRP54-type protein (amino acids 162-370 with reference to SEQ ID NO:2); and a P-loop nucleotide binding motif, (pre) (amino acids 164-183 with reference to SEQ ID NO:2). The helical bundle and GTPase domains are universally conserved in SRP-type receptor proteins, indicating a protein-binding function for the TLA2-CpFTSY protein. Upon GTP hydrolysis at the GTPase domain, the light-harvesting protein is integrated into the thylakoid membrane. GTP energy is required to reconstitute the soluble phase of light-harvesting chlorophyll protein transport into the thylakoid membrane. The P-loop nucleotide-binding motif contains the GTP binding motif, which is found in many nucleotide-binding proteins.

Other examples of Tla2 sequences include those from maize (GenBank Accession No. AJ549215) and Arabidopsis (GenBank Accession Nos. AY051026; AF360125). The Tla2 gene in Chlamydomonas was annotated as the FtsY gene based on sequence similarity (see, e.g., NW_{—}001843769.1: the website http followed by genome.jgi-psf.org/cgi-bin/dispTranscript?db-Chlre3&table=protein&id=105469&useCoords=1&withTranslation=1&dispRuler=1&width=90&padding=200; and the www site ncbi.nlm.nih.gov/nuccoreNW_{—}001843769.1?report=graph&from=6259&to=13510&strand=true&tracks).

The invention employs various routine recombinant nucleic acid techniques. Generally, the nomenclature and the laboratory procedures in recombinant DNA technology described below are those well known and commonly employed in the art. Many manuals that provide direction for performing recombinant DNA manipulations are available, e.g., Sambrook & Russell, Molecular Cloning, A Laboratory Manual (3rd Ed, 2001); and Current Protocols in Molecular Biology (Ausubel et al., eds., 1994-2011).

### TLA2 Nucleic Acid Sequences

Isolation or generation of Tla2 polynucleotide sequence can be accomplished by a number of techniques. For instance, oligonucleotide probes based on the sequences disclosed here can be used to identify the desired polynucleotide in a cDNA or genomic DNA library from a desired plant species. Such a cDNA or genomic library can then be screened using a probe based upon the sequence of a cloned Tla2 gene, e.g., SEQ ID NO:1 or 3. Probes may be used to hybridize with genomic DNA or cDNA sequences to isolate homologous genes in the same or different plant species.

Alternatively, the nucleic acids of interest can be amplified from nucleic acid samples using amplification techniques. For instance, PCR may be used to amplify the sequences of the genes directly from mRNA, from cDNA, from genomic libraries or cDNA libraries. PCR and other in vitro amplification methods may also be useful, for example, to clone nucleic acid sequences that code for proteins to be expressed, to make nucleic acids to use as probes for detecting the presence of the desired mRNA in samples, for nucleic acid sequencing, or for other purposes.

Appropriate primers and probes for identifying a Tla2 gene from plant cells, e.g., algae, can be generated from comparisons of the sequences provided herein. For a general overview of PCR see PCR Protocols: A Guide to Methods and Applications. (Innis, M, Gelfand, D., Sninsky, J. and White, T., eds.), Academic Press, San Diego (1990). Examples of primers are provided in the Examples section. Illustrative amplification reaction conditions are: 20 mM Tris HCl, pH 8.4, 50 mM potassium chloride. 2.5 mM magnesium chloride, 0.25 mM dATP, 0.25 mM dCTP. 0.25 mM dGTP, 0.25 mM dTTP, 0.6 M primers, and 2.5 units Taq polymerase/PCR reaction. An illustrative thermal cycling program is 94° C. for 3 min., 35 cycles of 95° C. for 45 sec, 55° C.-59° C. for 30 sec, 72° C. for 130 sec. followed by 72° C. for 10 min.

The genus of Tla2 nucleic acid sequences for use in the invention includes genes and gene products identified and characterized by techniques such as hybridization and/or sequence analysis using reference nucleic acid sequences, e.g., SEQ ID NOs: 1 and 3, and protein sequences, e.g., SEQ ID NO:2.

### Preparation of Recombinant Vectors

Recombinant DNA vectors suitable for transformation of green microalgae cells are employed in the methods of the invention. Preparation of suitable vectors and transformation methods are well known in the art. For example, a DNA sequence encoding a sequence to suppress Tla2 expression (described in further detail below), will preferably be combined with transcriptional and other regulatory sequences which will direct the transcription of the sequence from the gene in the intended cells of the transformed plant.

Regulatory sequences include promoters, which may be either constitutive or inducible. In some embodiments, a promoter can be used to direct expression of Tla2 nucleic acids under the influence of changing environmental conditions. Examples of environmental conditions that may effect transcription by inducible promoters include anaerobic conditions, elevated temperature, or the presence of light. Promoters that are inducible upon exposure to chemicals reagents are also used to express Tla2 nucleic acids. Other useful inducible regulatory elements include copper-inducible regulatory elements (Mett et al., Proc. Natl. Acad. Sci. USA 90:4567-4571 (1993): Furst et al., Cell 55:705-717 (1988)); tetracycline and chlor-tetracycline-inducible regulatory elements (Gatz et al., Plant J. 2:397-404 (1992); Röder et al., Mol. Gen. Genet. 243:32-38 (1994): Gatz, Meth. Cell Biol. 50:411-424 (1995)); ecdysone inducible regulatory elements (Christopherson et al., Proc. Natl. Acad. Sci. USA 89:6314-6318 (1992); Kreutzweiser et al., Ecotoxicol. Environ. Safety 28:14-24 (1994)); heat shock inducible regulatory elements (Takahashi et al., Plant Physiol. 99:383-390 (1992); Yabe et al., Plant Cell Physiol. 35:1207-1219 (1994); Ueda et al., Mol. Gen. Genet. 250:533-539 (1996)); and lac operon elements, which are used in combination with a constitutively expressed lac repressor to confer, for example, IPTG-inducible expression (Wilde et al., EMBO J. 11:1251-1259 (1992)). An inducible regulatory element also can be, for example, a nitrate-inducible promoter, e.g., derived from the spinach nitrite reductase gene (Back et al., Plant Mol. Biol. 17:9 (1991)), or a light-inducible promoter, such as that associated with the small subunit of RuBP carboxylase or the LHCP gene families (Feinbaum et al., Mol. Gen. Genet. 226:449 (1991); Lam and Chua, Science 248:471 (1990)), or a light.

In one example, a promoter sequence that is responsive to light may be used to drive expression of a Tla2 nucleic acid construct that is introduced into Chlamydomonas that is exposed to light (e.g., Hahn, Curr Genet 34:459-66, 1999; Loppes, Plant Mol Biol 45:215-27, 2001; Villand, Biochem J 327:51-7), 1997. Other light-inducible promoter systems may also be used, such as the phytochrome/PIF3 system (Shimizu-Sato, Nat Biotechnol 20):1041-4, 2002). Further, a promoter can be used that is also responsive to heat can be employed to drive expression in green microalgae such as Chlamydomonas (Muller, Gene 111:165-73, 1992; von Gromoff, Mol Cell Biol 9:3911-8, 1989). Additional promoters for expression in green microalgae include the RbcS2 and PsaD promoters (see, e.g., Stevens et al. Mol. Gen. Genet. 251: 23-30, 1996; Fischer & Rochaix, Mol Genet Genomics 265:888-94, 2001).

In some embodiments, the promoter may be from a gene associated with photosynthesis in the species to be transformed or another species. or example such a promoter from one species may be used to direct expression of a protein in transformed algal cells or cells of another photosynthetic marine organism. Suitable promoters may be isolated from or synthesized based on known sequences from other photosynthetic organisms. Preferred promoters are those for genes from other photosynthetic species that are homologous to the photosynthetic genes of the algal host to be transformed. For example, a series of light harvesting promoters from the fucoxanthing chlorophyll binding protein have been identified in Phaeodactylum tricornutum (see, e.g., Apt, et al. Mol Gen. Genet. 252:572-579, 1996). In other embodiments, a carotenoid chlorophyll binding protein promoter, such as that of peridinin chlorophyll binding protein, can be used.

In some embodiments, a promoter used to drive expression of a heterologous Tla2 gene is a constitutive promoter. Examples of constitutive strong promoters for use in green microalgae include, e.g., the promoters of the atpA, atpB, and rbcL genes. Other promoters that are operative in plants, e.g., promoters derived from plant viruses, such as the CaMV35S promoters, can also be employed in green microalgae algae.

In some embodiments, promoters are identified by analyzing the 5′ sequences of a genomic clone corresponding to the Tla2 genes described here. Sequences characteristic of promoter sequences can be used to identify the promoter. Sequences controlling eukaryotic gene expression have been extensively studied and include basal elements such as CG-rich regions, TATA consensus sequences etc. In plants, further upstream, there is typically a promoter element with a series of adenines surrounding the trinucleotide G (or T) N G. J. Messing et al., in GENETIC ENGINEERING IN PLANTS, pp. 221-227 (Kosage, Meredith and Hollaender, eds. (1983)).

A number of methods are known to those of skill in the art for identifying and characterizing promoter regions in plant genomic DNA (see, e.g., Jordano, et al., Plant Cell, 1: 855 866 (1989); Bustos, et al., Plant Cell, 1:839 854 (1989); Green, et al., EMBO J. 7, 4035 4044 (1988); Meier, et al., Plant Cell, 3, 309 316 (1991); and Zhang, et al., Plant Physiology 110: 1069 1079 (1996)). A promoter can be additionally evaluated by testing the ability of the promoter to drive expression in green microalgae cells in which it is desirable to introduce a Tla2 expression construct.

A vector comprising Tla2 nucleic acid sequences will typically comprise a marker gene that confers a selectable phenotype on algae cells. Such markers are known. For example, the marker may encode biocide resistance, particularly antibiotic resistance, such as resistance to zeocin, kanamycin, G418, bleomycin, hygromycin, or herbicide resistance, such as resistance to chlorosluforon or Basta. In some embodiments, selectable markers for use in Chlamydomonas can be markers that provide spectinomycin resistance (Fargo, Mol Cell Biol 19:6980-90, 1999), kanamycin and amikacin resistance (Bateman, Mol-Gen Genet 263:404-10, 2000), zeomycin and phleomycin resistance (Stevens, Mol Gen Genet 251:23-30, 1996), and paramomycin and neomycin resistance (Sizova, Gene 277:221-9, 2001).

A variety of different expression constructs, such as expression cassettes and vectors suitable for transformation of plant cells can be prepared. Techniques for transforming green microalgae are well known and described in the technical and scientific literature. The nuclear, mitochondrial, and chloroplast genomes of green microalgae can be transformed through a variety of known methods (see, e.g., Kindle, J Cell Biol 109:2589-601. 1989; Kindle, Proc Natl Acad Sci USA 87:1228-32, 1990; Kindle, Proc Natl Acad Sci USA 88:1721-5, 1991; Shimogawara, Genetics 148:1821-8, 1998; Boynton, Science 240:1534-8, 1988; Boynton, Methods Enzymol 264:279-96, 1996; Randolph-Anderson, Mol Gen Genet 236:235-44, 1993).

### Suppression of Tla2 Expression

The invention provides methods for generating a green microalgae having a reduced chlorophyll antenna size by suppressing expression of a nucleic acid molecule encoding Tla2. In a transgenic green microalgae of the invention, a nucleic acid molecule, or antisense constructs thereof, encoding a Tla2 gene product can be operatively linked to an exogenous regulatory element. The invention provides, for example, a transgenic green microalgae characterized by reduced chlorophyll antenna size having an expressed nucleic acid molecule encoding a Tla2 gene product, or antisense construct thereof, that is operatively linked to an exogenous constitutive regulatory element. In one embodiment, the invention provides a transgenic green microalgae that is characterized by small chlorophyll antenna size due to suppression of a nucleic acid molecule encoding a Tla2 polypeptide. Such a plant typically comprises an expression cassette stably transfected into the plant cell, such that that Tla2 polypeptide expression is inhibited constitutively or under certain conditions, e.g., when an inducible promoter is used.

Tla2 nucleic acid sequences can be used to prepare expression cassettes useful for inhibiting or suppressing Tla2 expression. A number of methods can be used to inhibit gene expression in green microalgae. For instance, siRNA, antisense, or ribozyme technology can be conveniently used. For example, in Chlamydomonas, antisense inhibition can be used to decrease expression of a targeted gene (e.g., Schroda, Plant Cell 11:1165-78, 1999). Alternatively, an RNA interference construct can be used (e.g., Schroda, Curr Genet. 49:69-84, 2006, Epub 2005 Nov. 25).

For antisense expression, a nucleic acid segment from the desired Tla2 gene is cloned and operably linked to a promoter such that the antisense strand of RNA will be transcribed. The expression cassette is then transformed into green microalgae and the antisense strand of RNA is produced. The antisense nucleic acid sequence transformed into plants will be substantially identical to at least a portion of the endogenous gene or genes to be repressed. The sequence, however, does not have to be perfectly identical to inhibit expression. Thus, an antisense or sense nucleic acid molecule encoding only a portion of Tla2 can be useful for producing a green microalgae in which Tla2 expression is suppressed. The vectors of the present invention can be designed such that the inhibitory effect applies to other proteins within a family of genes exhibiting homology or substantial homology to the target gene.

For antisense suppression, the introduced sequence also need not be full length relative to either the primary transcription product or fully processed mRNA. Generally, higher homology can be used to compensate for the use of a shorter sequence. Furthermore, the introduced sequence need not have the same intron or exon pattern, and homology of non-coding segments may be equally effective. Normally, a sequence of between about 30 or 40 nucleotides and about full length nucleotides should be used, though a sequence of at least about 100 nucleotides is preferred, a sequence of at least about 200 nucleotides is more preferred, and a sequence of at least about 500 nucleotides is especially preferred. Sequences can also be longer, e.g., 1000 or 2000 nucleotides are greater in length.

Catalytic RNA molecules or ribozymes can also be used to inhibit expression of Tla2 genes. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA cleaving activity upon them, thereby increasing the activity of the constructs.

A number of classes of ribozymes have been identified. One class of ribozymes is derived from a number of small circular RNAs that are capable of self-cleavage and replication in plants. Ribozymes, e.g., Group I introns, have also been identified in the chloroplast of green algae (see, e.g., Cech, Annu Rev Biochem 59:543-568, 1990; Bhattacharya, Molec Biol and Evol 13: 978-989, 1996; Erin, et al., Amer J Botany, 90:628-633, 2003; Turmel, et al., Nucl Acids Res. 21:5242-5250, 1993.; and Van Oppen et al., Molec Biol and Evol 10:1317-1326, 1993). The design and use of target RNA-specific ribozymes is described, e.g., in Haseloffer al. Nature, 334:585-591 (1988).

Another method of suppression is sense suppression (also known as co-suppression). Introduction of expression cassettes in which a nucleic acid is configured in the sense orientation with respect to the promoter has been shown to be an effective means by which to block the transcription of target genes. For an example of the use of this method to modulate expression of endogenous genes see, Napoli et al., The Plant Cell 2:279-289 (1990); Flavell, Proc. Natl. Acad. Sci., USA 91:3490-3496 (1994); Kooter and Mol, Current Opin. Biol. 4:166-171 (1993); and U.S. Pat. Nos. 5,034,323, 5,231,020. and 5,283.184.

Generally, where inhibition of expression is desired, some transcription of the introduced sequence occurs. The effect may occur where the introduced sequence contains no coding sequence per se, but only intron or untranslated sequences homologous to sequences present in the primary transcript of the endogenous sequence. The introduced sequence generally will be substantially identical to the endogenous sequence intended to be repressed. This minimal identity will typically be greater than about 65%, but a higher identity might exert a more effective repression of expression of the endogenous sequences. Substantially greater identity of more than about 80% is preferred, though about 90% or 95% to absolute identity would be most preferred. As with antisense regulation, the effect should apply to any other proteins within a similar family of genes exhibiting homology or substantial homology.

For sense suppression, the introduced sequence in the expression cassette, needing less than absolute identity, also need not be full length, relative to either the primary transcription product or fully processed mRNA. This may be preferred to avoid concurrent production of some plants that are overexpressers. A higher identity in a shorter than full length sequence compensates for a longer, less identical sequence. Furthermore, the introduced sequence need not have the same intron or exon pattern, and identity of non-coding segments will be equally effective. Normally, a sequence of the size ranges noted above for antisense regulation is used.

Endogenous gene expression may also be suppressed by means of RNA interference (RNAi), which uses a double-stranded RNA having a sequence identical or similar to the sequence of the target TLA2 gene. RNAi is the phenomenon in which when a double-stranded RNA having a sequence identical or similar to that of the target gene is introduced into a cell, the expressions of both the inserted exogenous gene and target endogenous gene are suppressed. The double-stranded RNA may be formed from two separate complementary RNAs or may be a single RNA with internally complementary sequences that form a double-stranded RNA. The introduced double-stranded RNA is initially cleaved into small fragments, which then serve as indexes of the target gene in some manner, thereby degrading the target gene. RNAi is known to be also effective in plants (see, e.g., Chuang, C. F. & Meyerowitz, E. M. Proc. Natl. Acad. Sci. USA 97: 4985 (2000); Waterhouse et al., Proc. Natl. Acad. Sci. USA 95:13959-13964 (1998): Tabara et al. Science 282:430-431 (1998)). For example, to achieve suppression of the expression of a DNA encoding a protein using RNAi, a double-stranded RNA having the sequence of a DNA encoding the protein, or a substantially similar sequence thereof (including those engineered not to translate the protein) or fragment thereof, is introduced into a plant of interest, e.g. green algae. The resulting plants may then be screened for a phenotype associated with the target protein and/or by monitoring steady-state RNA levels for transcripts encoding the protein. Although the genes used for RNAi need not be completely identical to the target gene, they may be at least 70%. 80%, 90%, 95% or more identical to the target gene sequence. See, e.g., U.S. Patent Publication No. 2004/0029283. The constructs encoding an RNA molecule with a stem-loop structure that is unrelated to the target gene and that is positioned distally to a sequence specific for the gene of interest may also be used to inhibit target gene expression. See, e.g., U.S. Patent Publication No. 2003/0221211.

The RNAi polynucleotides may encompass the full-length target RNA or may correspond to a fragment of the target RNA. In some cases, the fragment will have fewer than 100, 200, 300, 400, 500 600, 700, 800, 900 or 1,000 nucleotides corresponding to the target sequence. In addition, in some embodiments, these fragments are at least, e.g., 15, 20, 25, 30, 50, 100, 150, 200, or more nucleotides in length. In some cases, fragments for use in RNAi will be at least substantially similar to regions of a target protein that do not occur in other proteins in the organism or may be selected to have as little similarity to other organism transcripts as possible, e.g., selected by comparison to sequences in analyzing publicly-available sequence databases. Thus, RNAi fragments may be selected for similarity or identity with the N terminal region of the Tla2 sequences of the invention (i.e., those sequences lacking significant homology to sequences in the databases) or may be selected for identity or similarity to conserved regions of Tla2 proteins, e.g., the hydrophobic region.

Expression vectors that continually express siRNA in transiently- and stably-transfected cells have been engineered to express small hairpin RNAs, which get processed in vivo into siRNAs molecules capable of carrying out gene-specific silencing (Brummelkamp et al., Science 296:550-553 (2002), and Paddison, et al., Genes & Dev. 16:948-958 (2002)). Post-transcriptional gene silencing by double-stranded RNA is discussed in further detail by Hammond et al. Nature Rev Gen 2: 110-119 (2001), Fire et al. Nature 391: 806-811 (1998) and Timmons and Fire Nature 395: 854 (1998).

One of skill in the art will recognize that using technology based on specific nucleotide sequences (e.g., antisense or sense suppression technology), families of homologous genes can be suppressed with a single sense or antisense transcript. For instance, if a sense or antisense transcript is designed to have a sequence that is conserved among a family of genes, then multiple members of a gene family can be suppressed. Conversely, if the goal is to only suppress one member of a homologous gene family, then the sense or antisense transcript should be targeted to sequences with the most variation between family members.

### Screening for Plants Having Suppressed Tla2 Expression

The invention also provides methods of screening green microalgae having reduced Tla2 gene expression. Such plants can be generated using the techniques described above to target Tla2 genes. In other embodiments, mutagenized algae can be screened for reduced Tla2 gene expression.

Methods for introducing genetic mutations into green microalgae genes and selecting algae with desired traits are well known. For instance, green microalgae cells can be treated with a mutagenic chemical substance, according to standard techniques. Such chemical substances include, but are not limited to, the following: diethyl sulfate, ethylene imine, ethyl methanesulfonate and N-nitroso-N-ethylurea. Alternatively, ionizing radiation from sources such as, X-rays or gamma rays can be used. In other embodiments, insertional mutagenesis can be performed (see, e.g., Polle et al., Planta 217:49-59, 2003).

In other embodiments, insertional mutagenesis can be used to mutagenize a population of green algae that can subsequently be screened.

Green microalgae with mutations can be screened for decreased Tla2 gene expression. Such decreases are determined by examining levels of Tla2 gene or protein expression. Techniques for performing such an analysis are readily known in the art and include quantitative RT-PCR, northern blots, immunoassays, and the like. Tla2 expression can also be evaluated by analyzing a phenotypic endpoint such as chlorophyll antenna size and selecting plants having a smaller, or truncated, chlorophyll antenna size relative to normal.

### Uses of Tla2 Suppressed Algae

In the present invention, Tla2 is suppressed in algae. Algae, alga or the like, refer to plants belonging to the subphylum Algae of the phylum Thallophyta. The algae are unicellular, photosynthetic, anoxygenic algae and are non-parasitic plants without roots, stems or leaves; they contain chlorophyll and have a great variety in size, from microscopic to large seaweeds. Green algae (also referred to herein as green microalgae) are single cell eukaryotic organisms of oxygenic photosynthesis endowed with chlorophyll a and chlorophyll b belonging to Eukaryota—Viridiplantae—Chlorophyta—Chlorophyceae. Thus. for example, in some embodiments, Tla2 expression can be suppressed in C reinhardtii, which is classified as Volvocales—Chlamydomonadaceae. Other green microalgae that can be engineered to suppress Tla2 expression include Scenedesmus obliquus, Nannochloropsis, Chlorella, Botryococcus braunii, Botryococcus sudeticus, Dunaliella salina, and Haematococcus pluvialis.

Green microalgae can be used in high density photobioreactors (see, e.g., Lee et al., Biotech. Bioengineering 44:1161-1167, 1994; Chaumont, J Appl. Phycology 5:593-604, 1990), bioreactors for sewage and waste water treatments (e.g., Sawayama et al., Appl. Micro. Biotech., 41:729-731, 1994; Lincoln, Bulletin De L'institut Oceangraphique (Monaco), 12:109-115, 1993), elimination of heavy metals from contaminated water (e.g., Wilkinson, Biotech. Letters, 11:861-864, 1989), the production of β-carotene (e.g., Yamaoka, Seibutsu-Kogaku Kaishi, 72:111-114, 1994), the production of hydrogen (e.g., U.S. Patent Application Publication No. 20030162273), and pharmaceutical compounds (e.g., Cannell, 1990), as well as nutritional supplements for both humans and animals (Becker, 1993, “Bulletin De L'institut Oceanographique (Monaco), 12, 141-155) and for the production of other compounds of nutritional value.

Green microalgae that are engineered to suppress Tla2 expression in accordance with the invention may also be genetically modified with respect to other genes. For example, in some embodiments, the green microalgae may also comprises a heterologous isoprene synthase gene operably linked to a promoter (see, e.g., U.S. Pat. No. 7,947,478; WO 2008/003078) or to produce another product, e.g., that can be used to enhance production of ethanol or butanol.

Conditions for growing Tla2-suppressed algae for the purposes illustrated above are known in the art (see, e.g., the illustrative references cited herein).

Additional Plants that can be Tarted.

In a further aspect, the invention provides methods and compositions for suppressing Tla2 expression in other eukaryotic green plants where it is desirable to reduce the rate of light absorption. For example, crop plants, such as tobacco, soybeans, barley, maize, and others (see, e.g., Okabe, et al., J Plant Physiol. 60: 150-156, 1977; Melis & Thielen, Biochim. Biophys. Acta 589: 275-286, 1980; Ghirardi et al., Biochim. Biophys. Acta 851: 331-339, 1986; Ghirardi & Melis, Biochim. Biophys. Acta 932: 130-137, 1988; Droppa, et al., Biochim. Biophys. Acta 932: 138-145, 1988; and Greene. et al., Plant Physiol. 87: 365-370, 1988). As understood in the art, in such embodiments Tla2 expression is reduced. e.g., to a level of less than about 90%, 80%, 70%, 60%, 50%, 40%, 30%, or 20%, in genetically modified plants in which Tla2 is suppressed in comparison to unmodified plants, rather than eliminated. Methodology for reducing the level of expression and vectors that can be employed for this purpose are well known in the art, including using antisense, siRNA and other inhibitory methods as described, e.g., in U.S. Pat. No. 7,745,696.

### EXAMPLES

### Example 1

### Isolation of C. reinhardtii Strains with a Truncated Light-Harvesting Antenna Size (Tla Mutants)

A library of over 15,000 transformant stains was generated via DNA insertional mutagenesis of C. reinhardtii strain CC425 with linearized pJD67 plasmid (Davies et al., 1994). Exogenous DNA insertion into the genomic DNA of C. reinhardtii occurs randomly, occasionally interrupting nuclear-encoded genes, thus causing mutations. Transformant strains were initially isolated as arginine autotrophs, a property conferred upon transformation with pJD67 plasmid, as it contains a functional ARG7 gene (Davies et al., 1996). Arginine autotroph strains were screened as previously described (Polle et al., 2003). and also by measuring the Chl a/Chl b ratio of colonies in order to identify putative truncated light-harvesting antenna (tla) mutants. Out of the initial 15,000 transformants, six strains displayed a substantially higher Chl a/Chl b ratio than the corresponding parental wild type, indicating a putative smaller light-harvesting antenna size. One of these mutants, termed tla2, was selected for further analysis.

### Example 2

### Characterization of the Tla2 Mutant: Pigment Content and Composition

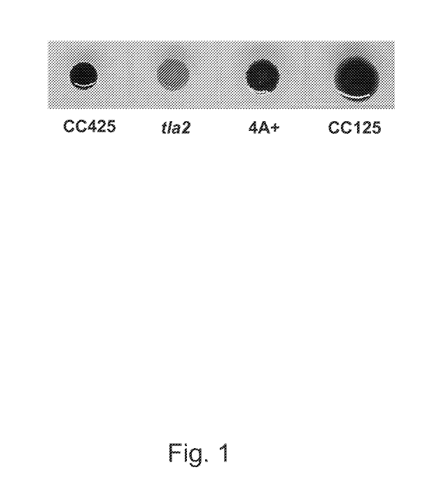

Cells of the tla2 strain, when cultivated as single cell colonies on agar, displayed lighter green coloration than their wild type counterparts (FIG. 1). Biochemical analysis showed that, on a per cell basis, the tla2 strain accumulated only about 20-25% of the chlorophyll present in the wild type. It also showed an elevated Chl a/Chl b ratio, suggesting lower amounts of the Chl a-b light-harvesting complex in the mutant (Table 1). The cellular content of Chl in wild type and tla2 was measured upon growth under two different light conditions: low light (30 μmol photons m⁻² s⁻) and medium light (450 μmol photons m⁻² s⁻¹) (Table I). Four wild type strains were used as controls for this analysis. Strain CC125 (ARG7, CW⁺) is the parental wild type strain of CC425 (arg2, cw15). Strain 4A+ (arg2, CW⁺) was used for backcrosses with the tla2 mutant. Strain CC503 (ARG7. cw⁻), was also employed, as this was applied by the JGI to the C. reinhardtii genome sequencing (Merchant et al., 2007). All wild type controls contained about 2.5 fmol Chl per cell under low light, and had a Chl a, Chl b ratio ranging between 2.7 and 3.0. The wild type Chl content per cell was lower when grown under medium light. In the wild type strains, it was about 1.7 fmol Chl per cell. A lower Chl/cell under medium light growth conditions is a compensatory response of the photosynthetic apparatus to the level of irradiance, seeking to balance the light and carbon reaction of photosynthesis (Greene et al., 1988: Smith et al. 1990).

TABLE I
Chlorophyll and carotenoid content and pigment ratios for wild type, tla2 mutant,
and tla2-complemented strains of Chlamydomonas reinhardtii (n = 3-5; means ± SD).
Low light [80 μmol photons m⁻²s⁻¹]

| | Chl/Cell | | Car/Cell | |
|---|---|---|---|---|
| Strain | [fmol] | Chl a/Chl b | [fmol] | Car/Chl |
| 4A+ | 2.57 ± 0.43 | 2.72 ± 0.07 | 1.07 ± 0.17 | 0.42 ± 0.00 |
| CC125 | 2.66 ± 0.13 | 3.00 ± 0.03 | 1.11 ± 0.06 | 0.42 ± 0.00 |
| CC504 | 2.36 ± 0.05 | 2.73 ± 0.05 | 0.93 ± 0.04 | 0.39 ± 0.01 |
| CC425 | 2.33 ± 0.10 | 2.86 ± 0.04 | 0.95 ± 0.04 | 0.41 ± 0.01 |
| C1 | 1.93 ± 0.17 | 2.87 ± 0.02 | 0.67 ± 0.16 | 0.42 ± 0.00 |
| C2 | 1.55 ± 0.04 | 3.01 ± 0.03 | 0.67 ± 0.00 | 0.43 ± 0.01 |
| C3 | 1.06 ± 0.01 | 3.35 ± 0.16 | 0.54 ± 0.01 | 0.51 ± 0.00 |
| C4 | 0.61 ± 0.09 | 3.92 ± 0.09 | 0.42 ± 0.05 | 0.68 ± 0.02 |
| tla2 | 0.46 ± 0.04 | 9.60 ± 0.98 | 0.38 ± 0.00 | 0.82 ± 0.06 |

Medium light [450 μmol photons m⁻²s⁻¹]

| | Chl/Cell | | Car/Cell | |
|---|---|---|---|---|
| Strain | [fmol] | Chl a/b | [fmol] | Car/Chl |
| 4A+ | 1.66 ± 0.37 | 2.45 ± 0.09 | 0.85 ± 0.17 | 0.51 ± 0.01 |
| CC125 | 1.85 ± 0.49 | 2.75 ± 0.14 | 1.01 ± 0.33 | 0.54 ± 0.04 |
| CC504 | 1.68 ± 0.33 | 3.08 ± 0.12 | 0.84 ± 0.13 | 0.50 ± 0.03 |
| CC425 | 1.35 ± 0.19 | 2.85 ± 0.04 | 0.74 ± 0.10 | 0.55 ± 0.01 |
| C1 | 1.03 ± 0.04 | 2.71 ± 0.09 | 0.56 ± 0.03 | 0.54 ± 0.02 |
| C2 | 0.71 ± 0.07 | 3.62 ± 0.11 | 0.52 ± 0.03 | 0.74 ± 0.03 |
| C3 | 0.51 ± 0.07 | 4.36 ± 1.05 | 0.43 ± 0.07 | 0.85 ± 0.07 |
| C4 | 0.35 ± 0.05 | 6.49 ± 0.56 | 0.34 ± 0.04 | 0.97 ± 0.02 |
| tla2 | 0.33 ± 0.04 | 7.92 ± 0.83 | 0.30 ± 0.03 | 0.90 ± 0.01 |

The tla2 mutant displayed a substantially lower Chl content per cell under both irradiance-growth conditions, which was equal to about 20% of that in the corresponding wild type controls: under low-light growth, it was about 0.5 fmol Chl/cell and under medium-light it was 0.3 fmol Chl/cell. The Chl a/Chl b ratio in the tla2 mutant was substantially greater than that of the wild type, and in the range of (8-10):1, reflecting absence of the auxiliary Chl b and possibly of a truncated light-harvesting Chl antenna size in this strain. The total carotenoid (Car) content in the tla2 mutant was lower relative to that in the wild type, albeit not in proportion to that of Chl. Consequently, the Car/Chl ratio was about 0.4-05:1 in the wild type strains and 0.8-0.9:1 in the tla2 mutant.

### Example 3

### Functional Properties and Chl Antenna Size Analysis of Wild Type and Tla2 Mutant

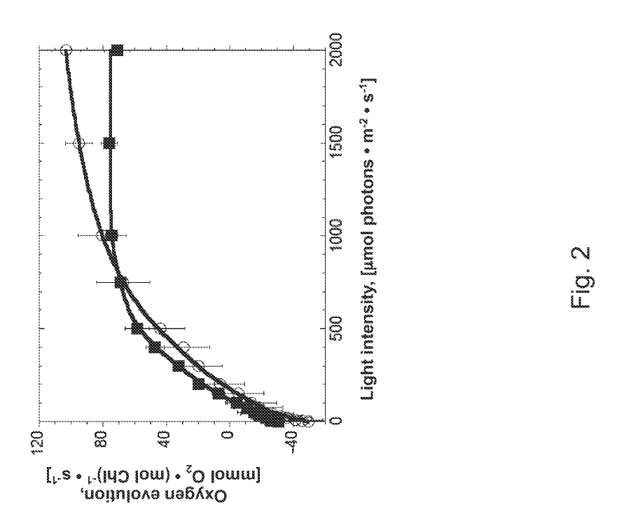

The functional properties of photosynthesis and the Chl antenna size of the tla2 mutant were assessed from the light-saturation curve of photosynthesis, i.e., from the relationship between light intensity and photosynthetic activity measured under in vivo conditions (Melis et al. 199; Polle et al., 2000; Polle et al., 2003). Light saturation curves of photosynthesis were measured with wild type and tla2 following cell acclimation to photoautotrophic growth at medium irradiance (growth at 450 μmol photons m⁻² s⁻). At zero incident intensity (in the dark) the rate of oxygen evolution was negative (FIG. 2). reflecting oxygen consumption by the process of cellular respiration (absence of photosynthesis). Measured on a per Chl basis, the rate of dark respiration of the tla2 mutant was about 50% greater than that of the wild type (FIG. 2). This higher rate of respiration is partially due to the lower Chl content per cell in the mutant. However, rates of respiration on a per cell basis were lower in the mutant, down to about 30% of those of the wild type (Table II).

TABLE II
Photosynthesis, respiration, and photochemical apparatus characteristics
of wild type (WT) and the tla2 mutant of Chlamydomonas reinhardtii grown
photo-autotrophically under medium light [450 μmol photons m⁻²s⁻¹]
conditions. Photosystem Chl antenna size and reaction center concentrations
were measured spectrophotometrically (Melis, 1998). (n = 3; means ± SD).

| Parameter measured | WT | tla2 |
|---|---|---|
| Respiration [mmol O_{2 ·} (mol Chl)⁻¹ _{·} s⁻¹] | 30.2 ± 11.9 | 49.1 ± 15.2 |
| Respiration [amol O_{2 ·} cell⁻¹ _{·} s⁻¹] | 55.8 ± 26.3 | 16.2 ± 5.4 |
| Quantum yield, relative units | 100 ± 25 | 108 ± 17 |
| Pₘₐₓ [mmol O_{2 ·} (mol Chl)⁻¹ _{·} s⁻¹] | 106.3 ± 12.8 | 152.3 ± 18.0 |
| Pₘₐₓ [amol O_{2 ·} cell⁻¹ _{·} s⁻¹] | 196.2 ± 46.2 | 50.3 ± 7.3 |
| Pₘₐₓ Respiration, relative units | 3.5 ± 1.9 | 3.1 ± 1.1 |
| Half-saturation intensity, | 210 | 380 |
| [μmol photons m⁻²s⁻¹] | | |
| Functional PSIIα Chl antenna size | 249 ± 27 | 160 ± 7 |
| Functional PSIIβ Chl antenna size | 90 ± 30 | 90 ± 12 |
| Fraction of PSIIα [%] | 61 ± 1 | 46 ± 1 |
| Average PSII Chl antenna size | 190 ± 20 | 120 ± 9 |
| Functional PSI Chl antenna size | 180 ± 9 | 123 ± 5 |

In the light-intensity region of 0-400 μmol photons m⁻² s⁻, the rate of photosynthesis increased as a linear function of light intensity, both in the wild type and tla2 mutant (FIG. 2). These linear portions of the light saturation curves were about parallel to one-another, suggesting similar quantum yields of photosynthesis for the two strains. This is an important consideration, as it shows that the tla2 mutation did not interfere with the high innate quantum yield of photosynthesis. The rate of photosynthesis in the wild type saturated at about 500 μmol photons m⁻² s⁻¹ (FIG. 2. solid circles), whereas that of the mutant continued to increase with light intensity through the 2,000 μmol photons m⁻² s⁻¹ level. From the average of several measurements, the light-saturated rate (Pₘₐₓ) for the wild type was about 100 mmol O₂ (mol Chl)⁻¹ s⁻¹, whereas Pₘₐₓ for the tla2 mutant was about 150 mmol O₂ (mol Chl)⁻¹ s⁻¹.

The light-saturated rate of photosynthesis is a measure of the overall photosynthetic capacity (Pₘₐₓ) (Powless and Critchley, 1980). A large wild-type light-harvesting Chl antenna causes saturation of photosynthesis at about 500 μmol photons m⁻² s⁻¹ (FIG. 2). A much higher light intensity of bright sunlight, >2000 μmol photons m⁻² s⁻¹, was needed to saturate photosynthesis in the tla2 mutant. Of note in the context of this experiment is the light intensity required to bring about the rate of photosynthesis to the half saturation point. The half-saturation intensity for the wild type was measured to be about 210 μmol photons m⁻²s⁻¹, while for the tla2 mutant it was 380 μmol photons m⁻²s⁻¹. As there is a reciprocal relationship between the half-saturation intensity of photosynthesis and the Chl antenna size. it can be concluded that photosystems (PSII & PSI) in the tla2 mutant collectively possess only about 55% the Chl antenna size found in the corresponding wild type. Such differences in the half-saturation intensity and Pₘₐₓ are typical among fully pigmented and truncated Chl antenna microalgae (Melis et al., 1999; Polle et al., 2000; Polle et al., 2003).

A more precise determination of the functional Chl antenna size of PSI and PSII units in wild type and the tla2 mutant was conducted by the spectrophotometric and kinetic method developed in this lab (Melis, 1989). The number of Chl molecules associated with each photosystem is given in Table II, measured in photoautotrophically grown cells under 450 μmol photons m⁻²s⁻¹. The number of Chl molecules of PSII_{α} and PSII_{β} was determined to be 250 and 90 for the wild type, respectively. These numbers were lowered to 160 and 90 for the tla2 mutant. The proportional abundance of PSII_{α} and PSII_{β} changed as a result of the mutation from 60:40 (PSII_{α}:PSII_{β}) in the wild type to 45:55 in the mutant. Thus, an average of 190 Chl molecules is associated with the reaction centers of PSII in the wild type, while the average PSII antenna size of the tla2 mutant was lowered to 120 Chl molecules (63%). The number of Chl molecules associated with a PSI reaction center was determined to be 210 for the wild type and 120 for the tla2 mutant. Thus, the PSI antenna size of the tla2 mutant was only about 60% of that in the wild type.

To investigate if the loss of photosystems and light-harvesting antenna affects photoautotrophic growth, the doubling time of tla2 in comparison with the wild type under medium-light (450 μmol photons m⁻² s⁻¹) conditions was measured. The doubling time of the wild type at this light intensity was determined to be 6.3±0.1 h. whereas the tla2 mutant doubled every about 7.2±0.3 h. This difference is consistent with the difference in the rate of oxygen evolution between the two strains at 450 μmol photons m⁻²s⁻¹ (FIG. 2).

The above functional and antenna analysis provided a foundation upon which the tla2 strain was deemed to be a good candidate for the identification of gene(s) impacting the Chl antenna size of the photosystems. Accordingly, a detailed molecular and genetic analysis was undertaken to map the plasmid insertion site and to test for plasmid and lesion co-segregation in the tla2 mutant, prior to gene cloning.

### Example 4

### Southern Blot Analysis of Wild Type and Tla2 Mutant

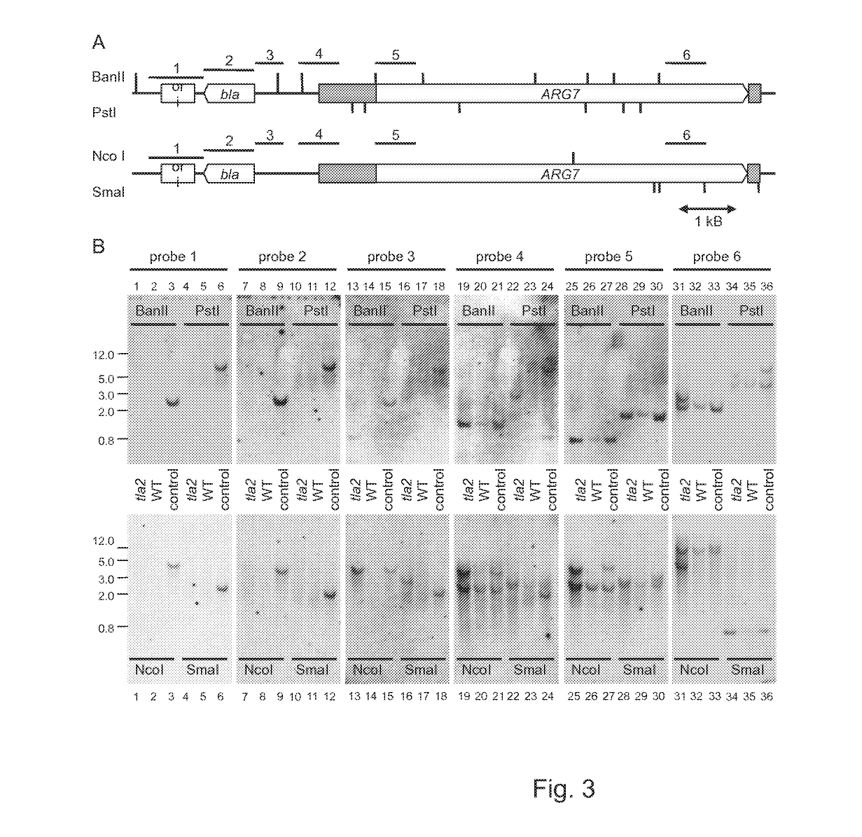

To determine the copy number of pJD67 insertions and their integrity in the tla2 mutant, Southern blot analysis of the tla2 genomic DNA was undertaken. A map of the linearized pJD67 plasmid. restriction sites on the pJD67 vector that were employed for the Southern blot analysis, the position of probes and their DNA hybridization regions are shown in FIG. 3A. Probes (1 through 6) were designed to various parts of the inserted pJD67 vector, as shown in FIG. 3A, and used to probe genomic DNA samples of tla2, its host wild type strain CC425, and a positive control (pJD67 transformant tla3 strain). Probes were selected for their specificity, with probe 1 being specific to the origin of replication of the pJD67 vector. Probe 2 covered the antibiotic resistance bla gene. Probe 3 was designed to hybridize to the intergenic region between the 3′ end of the bla gene and the ARG7 promoter region. Probe 4 covered both a plasmid specific sequence and the 5′ end of the ARG7 promoter region. The latter is present in both, the transforming plasmid and in the genomic DNA of the parental CC425 strain, as part of the endogenous inactive ARG7 gene. Probe 5 was designed to the 5′ coding region of the ARG7 gene. whereas probe 6 is specific to the 3′ end of the ARG7 gene. While Probes 1, 2 and 3 are specific to the pJD67 sequence, probes 4, 5 and 6 contain sequences that are also present in the C. reinhardtii host strain CC425 and thus at least one hybridization signal is expected to be generated by these latter probes.

Genomic C. reinhardtii DNA was digested with various restriction enzymes and size fractionated via agarose gel electrophoresis. Transfer to a positive-charged nylon membrane and hybridization reactions were carried out as shown in FIG. 3B. When tested with probe 1 or probe 2, tla2 genomic DNA digests did not generate a hybridization signal (FIG. 3B, lane 1 and 7). Absence of the ori and bla regions of the pJD67 plasmid from the tla2 DNA is consistent with the notion that the 5′ end of the inserted pJD67 plasmid is missing from the tla2 mutant. When tested with probe 3, a single hybridization band was detected, indicating a single insertion of the pJD67 vector into the genomic DNA of tla2 (FIG. 3B, lane 13 and 16). This finding was confirmed by using probes 4, 5 and 6. In theory, if 15 tla2 genomic DNA is hybridized with probes 4, 5 or 6, two distinct hybridization bands should be generated: one band originating from the exogenous pJD67 plasmid and one from the endogenous inactive ARG7 gene. This was found to be the case for digests with NcoI and SmaI using probe 4 and 5 (FIG. 3B, lanes 19 and 22 lower panel), and for BanII, PstI and NcoI digests upon using probe 6 (FIG. 3B, lanes 31 and 34). When probes 4 and 5 were used on BanII and PstI genomic DNA digests, only one hybridization band was detected. This is because these restriction enzymes generate fragments of about the same molecular weight from both the endogenous DNA sequence and the exogenously inserted pJD67 plasmid sequence (FIG. 3A). The corresponding restriction fragments using probe 5 were found to be 0.8 kb for the BanII digest and 1.6 kb for the PstI digest. The same applies to the fragment generated upon SmaI digest (0.8 kb) using probe 6. Using Probe 4 on BanII digests creates a fragment that is 1316 bp in size for the pJD67 insertion, and 1307 bp for the endogenous ARG7 gene sequence, as determined by a virtual digest of the published genome sequence (Merchant et al., 2007). These similar sizes could not be resolved on the agarose gel used for the Southern blot analysis, thus only a single hybridization band could be detected. In conclusion, the Southern blot analysis conducted here showed that genomic DNA of the tla2 mutant contains a single copy of the pJD67 inserted into the genomic DNA. and that about 2.5 kB of the pJD67 5′ end, including the ori and bla regions, have been deleted.

### Example 5

### Mapping the pJD67 Insertion Site in the Tla2 Genomic DNA

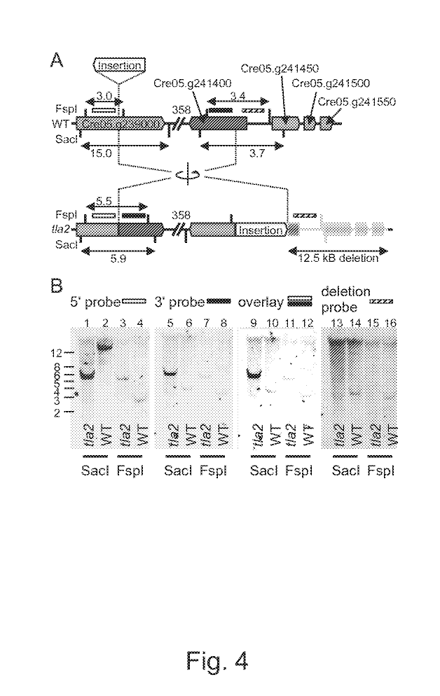

The Southern blot analysis revealed that the plasmid specific ori and bla loci at the 5′ end of the pJD67 plasmid are not present in the tla2 mutant. However, the downstream 5′ plasmid specific sequence was retained in the insertion site of the tla2 mutant, based on the fact that probe 3 generated a signal in the Southern blot of tla2 genomic DNA (FIG. 3B, lanes 13 and 16). By PCR analysis, using the same fixed-position reverse primer and shifted forward primer, it could be determined to a 20 bp accuracy, how much of the pJD67 vector has been retained in the tla2 mutant insert site. Subsequently, TAIL-PCR (Liu et al., 1995) was employed to amplify the genomic DNA flanking sequence on the 5′ of the insertion. The locus of the insertion was found to be within the coding sequence of a predicted gene Cre05.g239000 on the C. reinhardtii chromosome #5. Efforts to complement the mutant with BAC clones containing Cre05.g239000. namely BAC 28L08 and 21D17 failed: consequently the pJD67 insertion site in the tla2 mutant was further investigated. Analysis by TAIL-PCR, starting amplification upstream of the insertion site in the genomic DNA of the tla2 mutant and towards the insertion indicated that a 358 kb genomic DNA fragment flipped in orientation by 180 degrees (5′ to 3′) in the tla2 mutant. This unexpected finding was confirmed by Southern blot analyses using various restriction enzymes and two probes on both sides of the 5′ and 3′ of the flipped genomic DNA region (FIG. 4A, white and black bars). Restriction enzymes and probes were selected so that both probes would hybridize to DNA fragments of different sizes in genomic DNA digests of the wild type but would hybridize on the exact same fragment using genomic DNA digests of the tla2 mutant (FIG. 4A). In this approach, wild-type genomic DNA digests were expected to generate 3.7 and 15.0 kb (SacII), 3.4 and 3.0 kb (FspI) DNA fragments using the 5′ (FIG. 4A, white bar) and 3′ probe (FIG. 4A, black bar) for hybridization respectively. It was expected that use of these probes on tla2 mutant genomic DNA digests would generate hybridization signals from the exact same DNA fragment, i.e., 5.9 and 5.5 kb upon digestion with SacII and FspI, respectively.

Southern blots with the 5′ and 3′ end probe generated a single band in each lane of the expected size (FIG. 4B, lanes 1-8). The overlay of both blots (FIG. 4B, lanes 9-12) showed that the hybridization signals for the tla2 genomic digests are indeed at the exact same spot for both blots, while the wild type genomic DNA digests with SacI and FspI showed two separate hybridization signals. These results provide evidence that a 358 kb fragment of genomic DNA was broken off and subsequently reinserted in the opposite orientation (180 degree flip) during the process of the pJD67 insertion.

Further genomic DNA PCR analysis using various primers downstream from the flipped genomic DNA region revealed that a stretch 12.5 kb was deleted in the tla2 mutant (FIG. 4A). Included in this region were three predicted genes, namely Cre05.g241450, Cre05.g241500 and Cre05.g241550. To strengthen this finding, a probe in the putative deleted region was designed and was used in Southern blot DNA hybridization reactions (FIG. 4B, lanes 13-16). The probe clearly hybridized to fragments of wild type genomic DNA digested at expected sizes, 3.7 and 3.4 kb for SacI (FIG. 4B, lane 14) and FspI (FIG. 4B, lane 16), respectively. However, it failed to generate hybridization signals with the tla2 genomic DNA digests (FIG. 4B, lanes 13 and 15).

This detailed PCR and Southern blot analysis revealed that, in addition to the 180 degree flip of a 358 kb genomic DNA piece, four predicted genes in the tla2 mutant are affected either because of disruption or deletion, namely Cre05.g239000, Cre05.g241450, Cre05.g241500, and Cre05.g241550.

### Example 6

### Point of pJD67 Insertion is Linked with the Tla2 Phenotype

Genetic crosses were used to test if the point of pJD67 insertion is directly responsible for the tla2 phenotype. This is an important consideration, as the tla2 lesion may have occurred inadvertently in a locus distinct and far away from the pJD67 insertion site. To eliminate background mutations that do not contribute to the phenotype of tla2, progeny of the fourth cross of the original tla2 strain with arginine-requiring strain AG1-3.24 (arg2) were used in the below genetic crosses and PCR analysis.

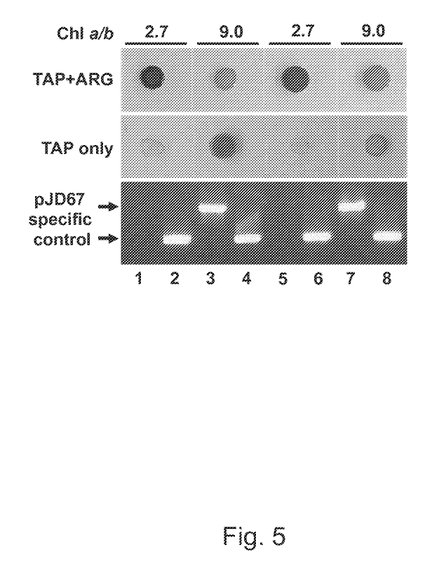

Ten complete tetrads were plated on non-selective media containing arginine (TAP+ARG) and on plates selective for the presence of a functional ARG7 gene within the insertion (TAP-only). FIG. 5 shows one typical tetrad analysis from such genetic crosses. When daughter cells were grown on TAP+ARG plates, the tetrad included two viable dark-green and two viable pale-green colonies (FIG. 5, upper panel). The dark green daughter cell colonies had a wild type Chl a/Chl b ratio (Chl a/Chl b=−2.7:1). A high Chl a/Chl b ratio (˜9:1) was measured for the pale green daughter cell colonies. A 2:2 wild type to tla2 phenotype distribution was found among the progeny of all tetrads that were tested, providing strong evidence that a single genetic locus is causing the tla2 phenotype. When plated on TAP-only agar plates (absence of arginine), the dark green daughter cells could not grow, apparently because they lacked a functional ARG7 gene and, therefore, lack arginine autotrophy (FIG. 5, middle panel). Cells able to grow on selective media were exclusively pale green progeny, indicating a linkage between the tla2 phenotype and the inserted pJD67 plasmid.

To further test whether the insertion locus is co-segregating with the tla2 phenotype, we used genomic DNA PCR analysis. A forward primer in predicted Cre05.g2390000RF and a reverse primer in the pJD67 sequence were employed. This combination of primers would generate a product only in the daughter cells of a genetic cross that actually carried the pJD67 insertion. As a positive control, a set of primers was used from a genomic DNA region of C. reinhardtii not affected by the insertion. FIG. 5 (lower panel) shows that dark green daughter cells failed to generate a pJD67-specific product (lanes 1 and 5) whereas they generated a product from the control primers (lanes 2 and 6). Pale green daughter cells, on the other hand, generated both an insertion-specific (lanes 3 and 7) and a control-specific product (lanes 4 and 8). These findings were observed with all tetrads examined. In conclusion, results of the PCR analysis are consistent with the genetic analysis and strongly suggest that a single locus is causing the tla2 phenotype and, furthermore, that it is linked with the pJD67 insertion site.

### Example 7

### Cloning of the TLA2 Gene

Using information from the sequenced C. reinhardtii genome and the BAC-end DNA we searched for BAC clones comprising the five deleted genes in the tla2 mutant. Two BAC clones, namely 28L06 and 21D17, were identified and shown to contain Cre05.g239000. Two other BAC clones, namely 08N24 and 36L15 were identified and shown to contain the genes Cre05.g241400 and Cre05.g241450. We could not identify a BAC clone that comprises genes Cre05.g241500 and Cre05.g241550. Each of the four identified BAC clones was used along with pBC1 (conferring paromomycin resistance) in a co-transformation approach to complement the tla2 strain. Transformants that grew on a paromomycin plate were screened for strains with a complemented tla2 phenotype. This was done upon measurement of the Chl/cell and the Chl a/Chl b ratio of the transformant colonies. Transformation with BAC clones 28L06 and 21D17 failed to generate any complemented strains. However, BAC clones 08N24 and 36L15 both successfully complemented the tla2 phenotype in about 50% of the co-transformed algae. The latter showed a dark green coloration and a low Chl a/Chl b ratio phenotype. BAC clones 08N24 and 36L15 contain two predicted C. reinhardtii genes, Cre05.g241400 and Cre05.g241450. These two genes were tested separately, as cDNA constructs, for their ability to complement the tla2 phenotype. For this purpose, the corresponding start and stop codon of the full length mRNA of both genes was identified by 5′ and 3′ RACE. Both cDNAs were then cloned separately into pSL18 (confers paromomycin resistance) for transformation of the tla2 mutant. Transformation with Cre05.g241400 cDNA did not yield any complemented strains, while the cDNA construct of gene Cre05.g241450 yielded complements in which the wild type phenotype was recovered to various degrees. These results suggested that deletion of gene Cre05.g241450 is responsible for the tla2 phenotype. Gene Cre05.g241450 is predicted to encode a putative FTSY precursor protein with a chloroplast-targeted transit peptide. A putative CpFTSY protein has not been previously reported or characterized in Chlamydomonas reinhardtii.

The CpFTSY gene of C. reinhardtii is 6578 bp long and consists of 13 exons and 12 introns (FIG. 6). The CpFTSY mRNA is 1814 bp in length with a 5′ and 3′ UTR of 189 and 479 bp, respectively (FIG. 6). The gene encodes for a protein of 381 amino acids including a putative 36 amino acid long chloroplast target peptide as determined by ChloroP (website www.cbs.dtu.dk/services/ChloroP/) and TargetP (website www.cbs.dtu.dk/servicesTargetP/) (FIG. 6). The mature protein of 345 amino acids with a molecular weight of 38.2 kD shares significant sequence homology with the SRP54_N helical bundle domain from amino acid 33-105 and the SRP54 type GTPase domain (amino acids: 126-333) as determined by Pfam (website pfam.sanger.ac.uk) (FIG. 6). These domains are universally conserved in SRP receptor proteins (Luirink and Sinning, 2004) indicating that Cre05.g241450 encodes for the CpFTSY protein.

### Example 8

### Complementation of the Tla2 Strain with the CpFTSY cDNA

Complementation of the tla2 strain with a wild type CrCpFTSY cDNA resulted in the isolation of several transformant lines, which showed various degrees of wild type recovery in their phenotype. The phenotypic complementation ranged anywhere between that of wild type and tla2 mutant. Some successfully transformed lines failed to rescue the mutation altogether. This variable effectiveness of the tla2 complementation is attributed to cDNA insertions in different regions of the chromosomal DNA in Chlamydomonas, many of which are either slow transcription zones, or are subject to epigenetic silencing. Four tla2-complemented lines were chosen for further detailed characterization, namely C1, C2, C3 and C4. Of those, C1 had a phenotype closest to the wild type, both in terms of the Chl/cell and Chl a/Chl b ratio (Table I). It was the best-complemented line out of the four lines investigated. It had a Chl a/Chl b ratio of 2.7-2.9 under either low or medium light conditions, which is in the same range as that of the wild type. The Chl/cell content of C1 was slightly lower under low light compared to the wild type strains with about 1.9 fmol Chl per cell. Under medium light this difference was exacerbated, with C1 cells containing about 1.0 fmol Chl, i.e., only about 60% of that in the wild type. However, under both low-light and medium-light growth conditions the Chl/cell in C1 was substantially greater than that in the tla2 mutant. C2, C3 and C4 lines were shown to be partially complemented strains of the tla2 mutant, with C2 having the highest and C4 the lowest Chl/cell, while the numbers for C3 were found to be in-between those of C2 and C4. This gradient of complementation from C1 to C4 was true for all photochemical apparatus parameters measured (Table I).

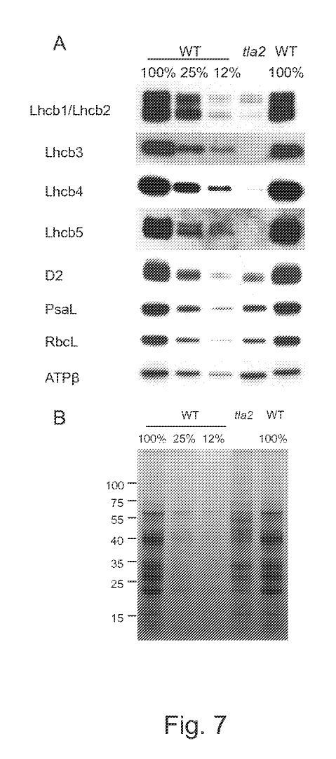

To further characterize the phenotype of the tla2 mutation in relation to wild type. Western blot analyses with specific PSII and LHC-II antibodies were performed (FIG. 7) using cells grown photoautotrophically under medium light. Lanes were loaded on an equal cell basis, except for the wild type where 25% (about equal reaction centers) and 12% cells were loaded additionally. All LHC-II proteins were either substantially lowered or not detected in the tla2 mutant. Lhcb1 and Lhcb2 were lowered the most to about 10% of wild type levels, while Lhcb3 was not detectable in the tla2 mutant. The minor antenna protein Lhcb4 was reduced to less then 5% of the wild type levels and no cross-reaction could be detected using an antibody raised against Lhcb5. The PSII reaction center protein D2 also showed a lower abundance on a per cell basis, down to about 20-25% of the wild type. However, loss of the peripheral Chl a-b antenna binding proteins is proportionally higher than the lowering of the photosystem reaction center proteins, consistent with the notion of a truncated light-harvesting antenna phenotype in the tla2 mutant. FIG. 7 further shows that the PSI reaction center protein PsaL is also lowered to about the same level as D2 (down to about 25% of that in the wild type). The same outcome pertains to the large subunit of RuBisCo (RbcL). The β subunit of the ATP-synthase (ATPβ) on the other hand was affected to a lesser extend by the loss of the CpFTSY protein in the tla2 mutant (FIG. 7).

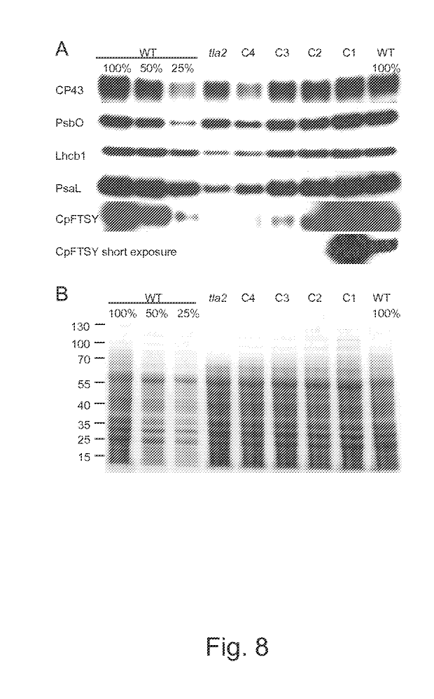

To test the level of CpFTSY protein expression in the wild type and tla2 complemented lines, Western blot analyses were conducted with specific polyclonal antibodies, directed against the recombinant CpFTSY protein of C. reinhardtii. No cross-reaction between CpFTSY-specific antibodies and a protein band at around 39 kD could be detected in the tla2 cell extracts, proving that tla2 is a knock-out mutant of CpFTSY (FIG. 8A). In the C4 complement, levels of the CpFTSY protein content were below 10% of those in the wild type, while C3 and C2 contained about 25% and 50% of the wild type CpFTSY protein, respectively. The C1 complemented line was found to substantially over-express the CpFTSY protein, as evident by the sizable dark band, seen even after a short film exposure in FIG. 8A. It was estimated that cells of the C1 complemented line accumulate more than a 5-fold CpFTSY protein than the wild type. However, this over-expression of the CpFTSY protein in the C1 complemented line did not increase the pigmentation of the cells in this strain, nor did it lower the Chl a/Chl b ratio to a value less than that of the wild type. This finding suggests that wild type levels of the CpFTSY protein are sufficient to meet all needs of the C. reinhardtii chloroplast and that levels of the CpFTSY protein in the wild type are not the limiting step in either the accumulation of Chl/cell or enhancement of the PS Chl antenna size.

Intermediate between wild type and tla2 values of the Chl/cell and the Chl a/Chl b ratio were observed in the C2-C4 complemented strains. These intermediate values correlated with the level of expression of the CpFTSY protein in these complemented lines. Accordingly, levels of expression for the CpFTSY protein, the Chl/cell and the reciprocal of the Chl a/Chl b ratio were in the order C1>C2>C3>C4 (see FIG. 8A and Table I).

Under low-light heterotrophic growth, the PSII reaction center proteins CP43 and PsbO accumulated in the tla2 mutant to about 50% of the wild type level, while the major PSII Chl a-b light-harvesting antenna protein Lhcb1 was lowered to a mere 10% of the wild type (FIG. 8A). The latter is consistent with the low pigmentation and also with the high Chl a/Chl b ratio of the tla2 mutant. The PSI reaction center protein PsaL was also found to be lower in abundance, down to about 10% in the tla2 mutant relative to the wild type. The different relative abundance of PSI and PSII in the tla2 mutant compared to the wild type under heterotrophic, low light growth versus photoautotrophic, medium light growth can be explained as a consequence of the changed light and growth conditions that prevail, rather than to a direct consequence of the tla2 mutation. The level of these proteins was restored in the C1 complemented line, while the other complemented lines showed intermediate protein contents directly correlating with the CpFTSY expression in these strains.

### Example 9

### CpFTSY is Localized in the Chloroplast Stroma

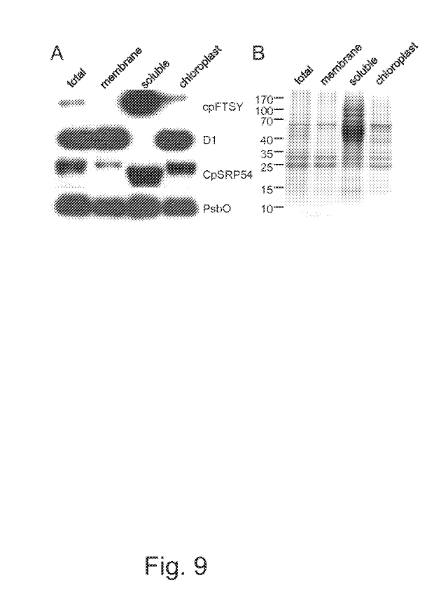

Two protein-targeting programs, namely TargetP and ChloroP, predicted chloroplast targeting of the precursor CpFTSY protein. The analysis with TargetP included a reliability score, which was rather low in the case of the CpFTSY apo-protein indicating a weak prediction. However, both programs predicted that the first 36 amino acids probably act as the chloroplast transit peptide. To investigate if the CpFTSY protein is indeed chloroplast localized, an intact chloroplast-enriched fraction was isolated from C. reinhardtii and probed by Western blot analysis (FIG. 9). Included in this analysis were proteins from total cell extract, thylakoid membrane fraction, soluble fraction of whole cells, and intact chloroplasts. For the total cell extract and the chloroplast-enriched fraction, equal amounts of chlorophyll was loaded. The Western blot analysis results showed that the amount of the CpFTSY protein was about the same in total cell and chloroplast extracts (FIG. 9A). CpFTSY antibody cross-reaction could be detected in the membrane fraction, whereas a strong CpFTSY cross-reaction was noted with proteins in the soluble extracts of C. reinhardtii. These results are consistent with predictions made on the basis of bioinformatic analysis (DAS, HMMTOP, PredictProtein, SOSUI, TMHMM, TMpred and TopPred software) assigning soluble properties to the CpFTSY protein. A similar profile was noted in Western blots of the above-mentioned protein extracts, probed with specific polyclonal antibodies raised against the CpSRPS4 protein of C. reinhardtii (FIG. 9A). The latter is postulated to function in tandem with the CpFTSY protein (see Discussion section). FIG. 9A also shows Western blot analysis results of the above mentioned protein extracts with specific polyclonal antibodies raised against the D1 and PsbO proteins of PSII, the latter serving as controls for the purity of the fractions that were employed in the localization of the CpFTSY protein.

### Example 10

### Chlorophyll-Protein Analysis of Wild Type and Tla2 Mutant by Non-Denaturing Deriphat-PAGE

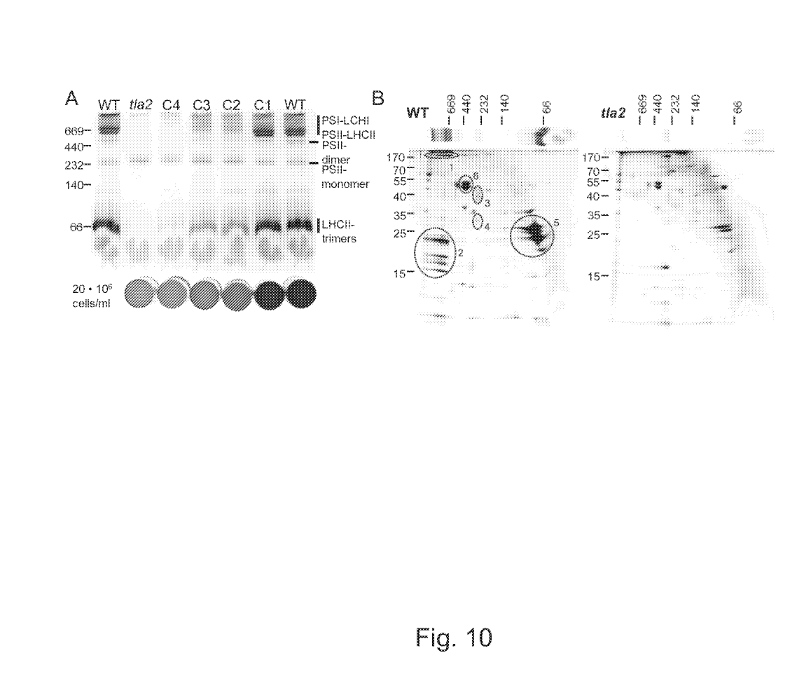

The pale green phenotype of the tla2 mutant, its low Chl content per cell, the higher than wild type Chl a/Chl b ratio, and the lower content of thylakoid membrane proteins (PSII-RC, Lhcb1, PSI-RC) all indicate alterations in the organization of the photosynthetic apparatus and in the light harvesting antenna of this strain. Further insight of these changes was afforded by non-denaturing Deriphat-PAGE analysis. In photosynthetic organisms the photosystems are organized in large complexes (holocomplexes) with the PS-core and LHC tightly coupled and integral to the thylakoid membrane. However, the subcomplexes can be separated by non-denaturing deriphat PAGE (Peter and Thornber, 1991). This method was used with thylakoid membrane preparations from tla2, its complemented C1-C4 lines and a wild type control. Four different pigment-containing protein complexes could be distinguished in the PAGE analysis of the wild type: large complexes, migrating to about 660 kD, PSI and PSII complexes, including their light harvesting antennae, PSII dimers (˜500 kD), PSII monomers (˜250 kD) and LHC-II trimers at around 70 kD (FIG. 10A). In the tla2 mutant most of these Chl-protein native bands were substantially reduced or absent. PSII-LHC-II super-complexes and PSII dimers could not be detected on the green native gels. On the other hand, the intensity of the PSII monomer band did not change significantly in the tla2 mutant relative to the wild type (lanes loaded on a per cell basis). In the tla2 complemented lines the green band attributed to PSII monomers stayed at about the same level, while all other green bands increased in their intensity in parallel with the degree of tla2-CpFTSY complementation.

A two-dimensional analysis of the protein complexes resolved by the native page was also undertaken (FIG. 10B). Putative proteins were identified based on their molecular weight in the 2-dimensional SDS-PAGE. Results obtained from the two-dimensional denaturing SDS-PAGE analysis were consistent with the notion of substantial depletion of the LHC from the tla2 mutant. The analysis further revealed that the abundance of the ATP synthase in tla2 thylakoids was also slightly lowered by the mutation since the alpha and beta subunits of this complex were not as abundant as those in the wild type (FIG. 10B).

### Example 11

### Phylogenetic Relationship

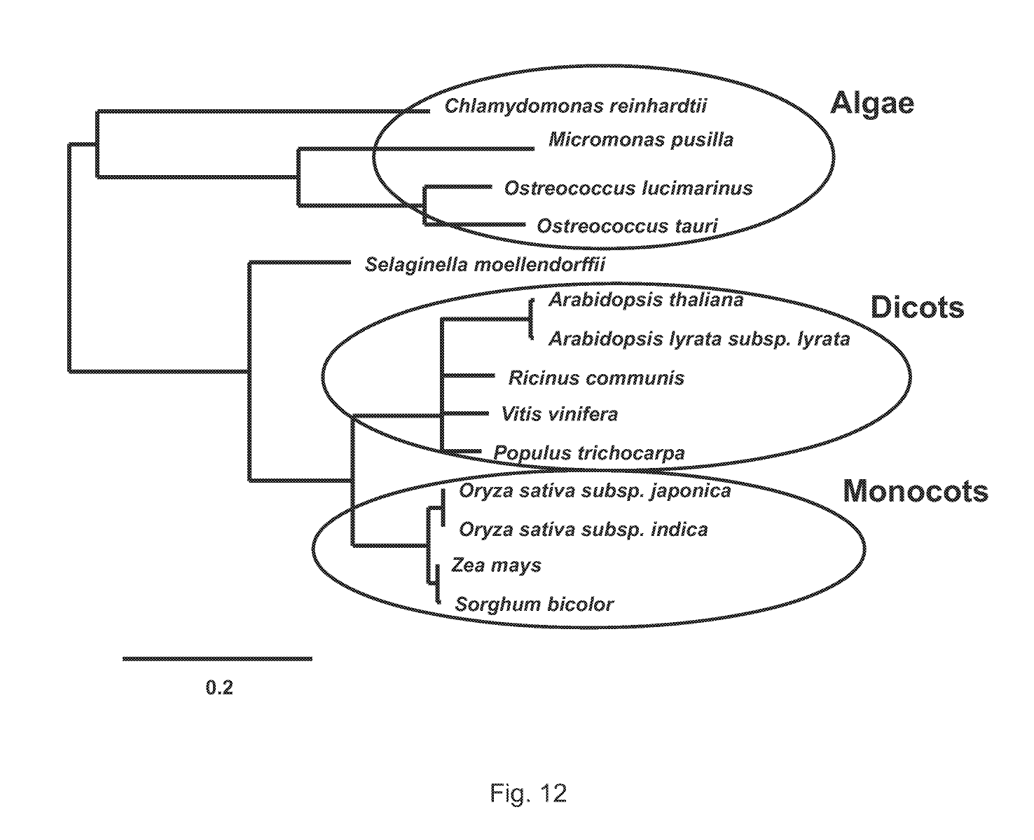

The maximum likelihood method was used to construct a phylogenetic tree displaying the evolutionary relationship of the TLA2-CpFTSY protein in photosynthetic eukaryotes based on their amino acid sequences (FIG. 12). The TLA2-CpFTSY proteins of algae form a distinct clade that is separate and apart from that of dicots (Arabidopsis through Populus) and monocots (Oryza through Sorghum), underlining the divergent functions of the TLA2-CpFTSY between algae and higher plants. Mosses (Selaginella) are intermediate to algae and plants.

### Discussion

The Chlamydomonas reinhardtii tla2 locus encodes for one of the components of the chloroplast Signal Recognition Particle (SRP), the nuclear-encoded and chloroplast-localized FTSY protein. This conclusion is based on the successful complementation of the tla2 mutant with a cDNA construct of the newly cloned CrCpFTSY gene. The product of the CrCpFTSY gene shares a sequence identity of about 54% with the CpFTSY protein of Arabidopsis thaliana and Zea mays, while the sequence identity of CpFTSY of these two plant species to each other is even greater, at 77%. This is not surprising considering the evolutionary distance between higher plants and green algae, but can explain differences in the plant versus algal phenotype of CpFTSY-deletion mutants. Earlier work in higher plants, i.e., pea (Tu et al., 1999), maize (Asakura et al., 2004) and Arabidopsis (Asakura et al. 2008) indicated that CpFTSY is either associated with the thylakoid membrane or equally partitioned between the soluble stroma and thylakoid membrane in the chloroplasts. However, the CrCpFTSY in this study was localized exclusively in the soluble chloroplast stroma fraction of Chlamydomonas reinhardtii. This difference could be explained in part by slightly varying properties of the plant versus algal CpFTSY, as evidenced by their amino acid sequence divergence. Another reason of the difference could be traced to differences in cell fractionation and thylakoid membrane isolation protocols between plants and unicellular green algae. In the latter, powerful sonication or French-press methods are employed in order to rupture the cell wall, an approach that invariably breaks the continuity of the thylakoid membrane in the chloroplast. Under these harsh mechanical fractionation conditions, it is possible that loosely bound CpFTSY proteins separate from the nascent thylakoid membranes.

### Role of CpFTSY in LHC Assembly

It has been reported that CpFTSY of higher plants is essential for the biogenesis of thylakoid membranes, including both the assembly of the Chl a-b light-harvesting complexes and that of the two photosystems (Asakura et al., 2008). CpFTSY is assumed to play a role in the correct integration of these transmembrane complexes in developing thylakoid membranes. Accordingly, cpftsy null mutants of higher plants could not grow photoautotrophically, as they lacked not only the LHC but also PSII and PSI. The deletion mutant of cpftsy in Chlamydomonas, as evidenced by the examples described above, showed a significantly different phenotype: most of the peripheral light-harvesting antenna complexes of PSI and PSII did not accumulate in the thylakoid membrane. However, and contrary to the observation of seedling-lethal cpftsy mutants in higher plants, the tla2 mutant of C. reinhardtii grew well photoautotrophically with a quantum yield of photosynthesis similar to that of the wild type. This substantially different property of the CrCpFtsY gene in green microalgae permits application of the CrCpFtsY gene in the generation of green microalgal strains with a truncated light-harvesting antenna (TLA) phenotype, useful in commercial applications comprising biomass, biofuels, and industrial chemicals production.

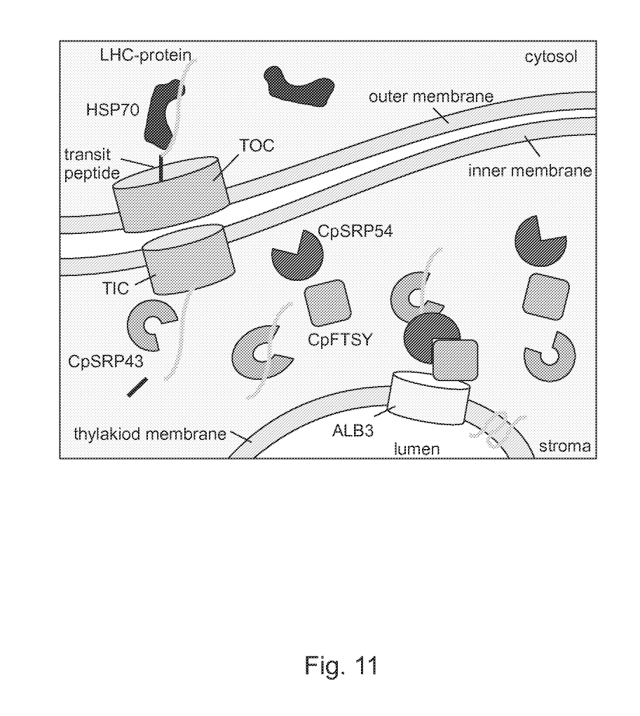

The CpFTSY in green microalgae plays a role in the integration of the photosystem-peripheral light-harvesting complexes into the thylakoid membrane. Not to be bound by theory, it presumably functions together with the other SRP pathway proteins CpSRP54, CpSRP43 and ALB3 (FIG. 11). CpSRP43 was shown to be a specific chaperon for light-harvesting proteins and plays a role to prevent and dissolve aggregation of the hydrophobic domains of the light-harvesting proteins after import into the chloroplast (Falk and Sinning, 2010; Jaru-Ampompan et al., 2010). CpSRP54 and CpFTSY are thought to bind to this LHC-protein/CpSRP43 complex and guide it to the membrane-bound translocase ALB3. Again not to be bound by theory, we suggest that the ALB3 translocase is specifically localized in the “polar” regions of the chloroplast, where the thylakoid biogenesis occurs. There, it receives the LHC-CpSRP43-CpSRP54-CpFTSY complex and guides the LHC in the nascent thylakoid membrane lipid bilayer. This concept of the localization of the LHC assembly apparatus is opposite to the notion of a uniform distribution of the ALB3 translocase throughout the entire thylakoid membrane. Upon GTP hydrolysis, the LHC-protein is integrated into the thylakoid membrane (Tu et al., 1999). SRP-proteins CpSRP54 and ALB3 are needed for the proper integration of other transmembrane proteins, as evident by the phenotype generated in the corresponding knockout mutants (Amin et al. 1999; Bellafiore et al. 2002).

Not to be bound by theory, it is proposed that the CpFTSY in green algae is most important in the integration of the light harvesting proteins, in contrast to higher plants where it appears to be involved in the integration of other transmembrane proteins in the thylakoid membrane as well (Asakura et al., 2004; Asakura et al., 2008). This is supported by the fact that the tla2 mutants grow well autotrophically and have functional PSII and PSI reaction centers. The lower reaction center protein content of the tla2 strain compared to a wild type is an indication of overall lower thylakoid membrane abundance in the chloroplast of the mutant. Not to be bound by theority, inability to assemble the imported light-harvesting proteins in the tla2 mutant may trigger a feedback inhibition in chlorophyll biosynthesis, indirectly affecting the chlorophyll supply and lowering the chloroplast ability to assemble the full amount of PSII and PSI reaction centers. In spite of the total absence of the CpFTSY, the tla2 mutant retained assembly activity for some of the light-harvesting proteins. It has been reported that chaperon CpSRP43, alone is sufficient to form a complex with the translocase ALB3 (Bals et al. 2010) and this could suffice to explain the observation that some light-harvesting antenna proteins can still become incorporated into the thylakoid membrane, albeit with a much lower efficiency.

It is of interest that rates of cellular respiration in the tla2 mutant were substantially lower, down to about 30% of those measured in the wild type. A truncated Chl antenna in the photosynthetic apparatus should not a priori affect the cell's respiration capacity (Polle et al., 2000; Polle et al., 2003). Rather, loss of respiration fitness in the tla2 mutant, and possibly also loss of PSII and PSI content, could be attributed to the loss of a number of genes flanking the pJD67 insertion site, which were deleted or rearranged in the tla2 genomic DNA. The deleted genes and the 358 kb genomic DNA 1800 flip are proximal to the insertion site and, therefore, could not be recovered in spite of the many crosses of the original tla2 strain with a wild type counterpart. The deleted genes, and those contained in the 358 kb 1800 flip, are predicted open reading frames of unknown function, and were not further analyzed in this work.

There are current on-going efforts to renewably generate fuel and chemical products for human consumption, through the process of microalgal photosynthesis. Such bio-products include H₂ and other suitable biofuel molecules (Melis, 2007; Hankamer et al., 2007; Greenwell et al., 2010; Hu et al., 2008; Mata et al., 2010), antigens (Dauvillee et al., 2010, Michelet et al., 2011) and high value bio-products (Mayfield, 2007). Sunlight energy conversion in photosynthesis must take place with the utmost efficiency, as this would help to make renewable fuel and chemical processes economically feasible. In plants and algae, the solar energy conversion efficiency of photosynthesis is thus a most critical factor for the economic viability of renewable fuel and chemical production (Melis, 2009). It has been shown that high-density cultures of algae with a truncated Chl antenna size are photosynthetically more productive under bright sunlight, due to the elimination of over-absorption and wasteful dissipation of excess energy (Nakajima and Ueda, 1997; 1999; Melis et al., 1999; Polle et al., 2003; Melis, 2009). The tla2 mutant has a permanently truncated light-harvesting antenna size phenotype and, in spite of a few collateral mutations in the plasmid insertion region, it shows a higher per chlorophyll photosynthetic productivity that the wild type cells.

The smaller light-harvesting Chl antenna size in the tla2 mutant requires a higher intensity to saturate photosynthesis than that in the wild type (FIG. 2). Thus, under limiting irradiance conditions in the wild, individual tla2 cells would be at a disadvantage over their wild type counterparts, as their light-harvesting ability has been compromised. It follows that the tla2 mutant strain, if released in the environment, would not be able to compete with fully pigmented strains and thus cannot survive. In high-density cultures, however, at light intensities greater than 800 μmol photons m⁻² s⁻¹, the tla2 mutant strains would collectively show greater productivity than their wild type counterparts, due to elimination of over-absorption and wasteful dissipation of sunlight by the former. Accordingly, application of the CpFTSY gene in tla2-type of mutations in microalgae can serve to minimize the ability of individual cells to over-absorb sunlight but at the same time helping to substantially improve the productivity of the overall mass culture.

### Materials and Methods

### Cell Cultivation

Chlamydomonas reinhardtii strains CC503, CC425. CC125 obtained from the Chlamydomonas Center (http://www.chlamy.org/), and laboratory strains 4A+ and tla2 were maintained under orbital shaking in 100 ml liquid cultures in Erlenmeyer flasks at 25° C. under continuous illumination at low light (30 μmol photons m⁻² s⁻¹). Irradiance was provided by balanced cool-white and warm-white fluorescent lamps. Cells were grown photo-heterotrophically in TAP medium (Gorman and Levine, 1965), or photo-autotrophically in HS medium (Harris, 1989) under a combination of cool-white, warm-white fluorescent, and incandescent irradiance at a light intensity of 450 μmol photons m⁻² s⁻¹. For physiological measurements, cultures were harvested during the logarithmic growth phase (˜1-3×10⁶ cells/ml).

### Cell Count and Chlorophyll Determination

Cell density was measured using an improved Neubauer ultraplane hemacytometer and a BH-2 light microscope (Olympus, Tokyo). Pigments from intact cells or thylakoid membranes were extracted in 80% acetone and cell debris removed by centrifugation at 20,000 g for 5 min. The absorbance of the supernatant was measured with a Shimadzu UV-1800 spectrophotometer, and the chlorophyll concentration of the samples was determined according to Arnon (1949), with equations corrected as in Melis et al. (1987).

### Mutagenesis and Screening Protocols

Mutants of Chlamydomonas reinhardtii were obtained upon DNA insertional mutagenesis and transformation by the glass-bead method, as described in Debuchy et al. (1989). Parental strain CC425, an arginine auxotroph, was transformed with 1 mg HindIII linearized plasmid pJD67, containing the structural gene (ARG7) of the argininosuccinate lyase to complement the arginine requiring phenotype of the CC425 strain (Davies et al., 1994; 1996). Wild type strain CC503 was transformed with 0.5 μg of KpnI linearized pBC1 plasmid (a gift from the lab of Dr. Krishna Niyogi) conferring paromomycin resistance. Transformants were selected on TAP-only media and initially screened upon measurement of the Chl a/Chl b ratio of the strains, following extraction of chlorophyll with 80% acetone. A Biotek Epoch (USA) spectrophotometer equipped with a 96-well plate-reader was used in these measurements.

### Nucleic Acid Extractions

Chlamydomonas reinhardtii genomic DNA was isolated for PCR analysis using Qiagen's Plant DNA purification kit. For Southern blot analysis, genomic DNA was isolated by harvesting cells from a 50 ml aliquot of the culture upon centrifugation at 5000 g for 5 min. followed by re-suspension of the pellet in 500 ml sterile water. Cells were lysed upon addition of 500 ml lysis buffer containing 2% SDS, 400 mM NaCl, 40 mM EDTA, 100 mM Tris/HCl (pH 8.0) and upon incubation for 2 h at 65° C. To this mix, 170 ml of 5 M NaCl solution was added. SDS and carbohydrates where precipitated upon addition of 135 ml 10% CTAB in 0.7 M NaCl and incubation for 10 min at 65° C. They were extracted by mixing with Chloroform:Isoamylalcohol 24:1 followed by centrifugation at 20,000 g for 5 minutes. Proteins were removed by extraction with Phenol:Chloroform:Isoamylalcohol 25:24:1 solution. RNA in the water-phase was digested using 10 ng/ml RNase A and upon incubation at 37° C. for 20 min. RNase A was removed by extraction with Phenol:Chloroform:Isoamylalcohol 25:24:1 solution. Desalting of the DNA solution was achieved upon precipitation with Isopropanol and resuspension in 10 mM Tris-HCl (pH 8.0).

### Southern Blot Analysis

Approximately 5 mg genomic DNA was digested by various restriction enzymes (NEB) in a 500 ml volume at 37° C. with overnight incubation (16 mh). The digested DNA was precipitated with isopropanol, washed in 70% ethanol and resuspended in 20 ml buffer containing 5 mM Tris, pH 8.0. DNA fragments were separated on a 0.6% agarose gel, transferred on a positively charged nylon membrane (Hybond-N⁺; Amersham) and UV cross-linked. Probes were obtained upon PCR reactions using specific primers (Table 3) and the pJD67 plasmid as template DNA, and labeled with alkaline phosphatase using the “Gene Images AlkPhos Direct Labeling and Detection System” kit (Amersham). The manufacture's protocol was used for labeling, hybridization, washing and signal detection with the following modifications: hybridization temperature and primary washing buffer temperature was maintained at 72° C.

TABLE 3
Primers

| | | | | anneal. |
|---|---|---|---|---|
| | | | restriction | temp. |
| Primer | Sequence | description | site | (° C.) |
| K001 | ATTGGGCGCTCTT | generates with HK002 and pJD67 as template DNA | | 60.7 |
| | CCGCTTC | probe 1 in FIG. 3 | | |
| K002 | GCCTCACTGATTA | generates with HK001 and pJD67 as template DNA | | 54.0 |
| | AGCATTGG | probe 1 in FIG. 3 | | |
| K003 | TATGAGTAAACTT | generates with HK004 and pJD67 as template DNA | | 52.0 |
| | GGTCTGACAG | probe 2 in FIG. 3 | | |
| K004 | AGGAAGAGTATG | generates with HK003 and pJD67 as template DNA | | 49.3 |
| | AGTATTCAAC | probe 2 in FIG. 3 | | |
| K081 | GGAATAAGGGCG | generates with HK082 and pJD67 as template DNA | | 59.6 |
| | ACACGGAAATGTT | probe 3 in FIG. 3 | | |
| K082 | CTCCTTTCGCTTTC | generates with HK081 and pJD67 as template DNA | | 59.4 |
| | TTCCCTTCCTTTC | probe 3 in FIG. 3 | | |
| K094 | CTAGAACTAGTGG | generates with HK095 and pJD67 as template DNA | | 58.2 |
| | ATCCCCCGAAC | probe 4 in FIG. 3 | | |
| K095 | CTCATCCTCCTCG | generates with HK094 and pJD67 as template DNA | | 59.5 |
| | CACTCGTG | probe 4 in FIG. 3 | | |
| K096 | GTTACAAGCGACG | generates with HK097 and pJD67 as template DNA | | 57.0 |
| | AATGCGTG | probe 5 in FIG. 3 | | |
| K097 | CTGTGCCGCACCT | generates with HK096 and pJD67 as template DNA | | 59.1 |
| | TGATGTC | probe 5 in FIG. 3 | | |
| K038 | GTTTGTGCAGGAG | generates with HK039 and pJD67 as template DNA | | 58.5 |
| | TGTTGGGAG | probe 6 in FIG. 3 | | |
| K039 | AACGTTCGATAGC | generates with HK038 and pJD67 as template DNA | | 54.9 |
| | TCTCACAAC | probe 6 in FIG. 3 | | |
| K120 | CCACTACGTGAAC | fine mapping of pJD67 sequence in tla2, generates a | | 58.8 |
| | CATCACCCTAATC | produce with tla2 gDNA template with HK082 | | |
| K119 | AGACCGAGATAG | fine mapping of pJD67 sequence in tla2, generates a | | 59.3 |
| | GGTTGAGTGTTGT | produce with tial gDNA template with HK082 | | |
| K118 | CAAATAGGGGTTC | fine mapping of pJD67 sequence in tla2, generates a | | 59.2 |
| | CGCGCAC | produce with tla2 gDNA template with HK082 | | |
| K081 | GGAATAAGGGCG | fine mapping of pJD67 sequence in tla2, generates no | | 59.6 |
| | ACACGGAAATGTT | produce with tla2 gDNA template with HK082 | | |
| K082 | CTCCTTTCGCTTTC | fine mapping of pJD67 sequence in tla2 | | 59.4 |
| | TTCCCTTCCTTTC | | | |
| K030 | NTCGWGTSCNA | arbitrary degenerate primers for TAIL PCR | | |
| | GC | | | |
| K031 | WGNTCWGNCANG | arbitrary degenerate primers for TAIL PCR. | | |
| | CG | | | |
| K121 | GACGGTTTTTCGC | TAIL PCR primary reaction, between bla and and ARG7 | | 64.4 |
| | CCTTTGACGTTGG | of pJD67 | | |
| | AGTC | | | |
| K122 | CACGTTCTTTAAT | TAIL PCR secondary reaction, between bla and and | | 54.9 |
| | AGTGGACTCTTGT | ARG7 of pJD67 | | |
| K123 | CCAAACTGGAACA | TAIL PCR tertiary reaction, between bla and and ARG7 | | 50.1 |
| | ACACT | pJD67 | | |
| K124 | AACGCGAATTTTA | TAIL PCR primary reaction, between bla and and ARG7 | | 58.8 |
| | ACAAAATATTAAC | of pJD67 | | |
| | GCTTACAATTTAG | | | |
| | G | | | |
| K125 | TGGCACTTTTCGG | TAIL PCR secondary reaction, between bla and and | | 57.6 |
| | GGAAATGT | ARG7 of pJD67 | | |
| K126 | GGAACCCCTATTT | TAIL PCR tertiary reaction, between bla and and ARG7 | | 50.6 |
| | GTTTATTTTTC | of pJD67 | | |
| K169 | GTCCAACATGGGC | TAIL PCR primary reaction, chromosome 5, 5′ of | | 64.4 |
| | GACCGCATCTG | insertion | | |
| K170 | CATGCCGGACCCG | TAIL PCR secondary reaction, chromosome 5, 5′ of | | 58.5 |
| | TTAACATC | insertion | | |
| K171 | CTCCACCAACGTC | TAIL PCR tertiary reaction, chromosome 5, 5′ of | | 55.8 |
| | ACCATC | insertion | | |
| K214 | GTTTATCAGATTG | generates with HK215 and WT C. reinhardtii gDNA as | | 60.5 |
| | AGAGTGAAACTGG | template DNA 3′ probe in FIG. 4 | | |
| | CGCTTTAC | | | |
| K215 | CATGACCTACTCG | generates with HK214 and WT C. reinhardtii gDNA as | | 59.7 |
| | GCTCGCATTC | template DNA 3′ probe in FIG. 4 | | |
| K131 | CAAAACCTCACCG | generates with HK132 and WT C. reinhardtii gDNA as | | 59.6 |
| | TGGATTTCGTCAA | template DNA 5′ probe in FIG. 4 | | |
| K132 | GGGTTGTAATACC | generates with HK131 and WT C. reinhardtii gDNA as | | 59.5 |
| | GTGGGGATTTTAA | template DNA 5′ probe in FIG. 4 | | |
| | GC | | | |
| K172 | GTAGACTATCCTC | generates with HK173 and WT C. reinhardtii gDNA as | | 60.0 |
| | CGCTTTGAATCAA | template DNA deletion probe in FIG. 4 | | |
| | GGTG | | | |
| K173 | CACGAGGTGTTTG | generates with HK172 and WT C. reinhardtii gDNA as | | 59.6 |
| | ACAGTTTGACTGA | template DNA deletion probe in FIG. 4 | | |
| | AG | | | |
| K193 | GCTTACGTGGAGC | crude deletion mapping on chromosome 5 in tla2, 10.1 | | 59.1 |
| | GTGCAG | kb downstream of insertion. No product with HK194 | | |
| | | using tla2 gDNA. | | |
| K194 | CAGTTTACTGGTG | crude deletion mapping on chromosome 5 in tla2, 10.1 | | 60.2 |
| | TCGTGGTGCAC | kb downstream of insertion. No product with HK193 | | |
| | | using tla2 gDNA. | | |
| K216 | CACCCAACCTCAC | crude deletion mapping on chromosome 5 in tla2, 11.7 | | 60.5 |
| | GACCACAC | kb downstream of insertion. No product with HK216 | | |
| | | using tla2 gDNA. | | |
| K217 | CACCCCTCCGTGT | crude deletion mapping on chromosome 5 in tla2, 11.7 | | 61.1 |
| | TGTAACCTATACA | kb downstream of insertion. No product with HK216 | | |
| | ACTAC | using tla2 gDNA. | | |
| K220 | GTGGCACTTTTAG | crude deletion mapping on chromosome 5 in tla2, 13.8 | | 59.7 |
| | TGTCAAGCATACT | kb downstream of insertion. Gives product with HK216 | | |
| | GTG | using tla2 gDNA. | | |
| K221 | GGATGCGGAATCC | crude deletion mapping on chromosome 5 in tla2, 13.8 | | 60.7 |
| | ACCCACAATG | kb downstream of insertion. Gives product with HK216 | | |
| | | using tla2 gDNA. | | |
| K220 | GTGGCACTTTTAG | fine deletion mapping on chromosome 5 in tla2. Gives | | 59.7 |
| | TGTCAAGCATACT | product with HK240 using tla2 gDNA. | | |
| | GTG | | | |
| K238 | CCTGAACAAGGGT | fine deletion mapping on chromosome 5 in tla2. Gives | | 59.2 |
| | GCATGCATG | product with HK240 using tla2 gDNA. | | |
| K237 | GTCGTGCGCGTTT | fine deletion mapping on chromosome 5 in tla2. No | | 59.5 |
| | CAGGTC | product with HK240 using tla2 gDNA. | | |
| K236 | CTTCGGAACCATT | line deletion mapping on chromosome 5 in tla2. No | | 60.1 |
| | GTTGATTACACTT | product with HK240 using tla2 gDNA. | | |
| | CTATAAAGCAG | | | |
| K240 | CACCTTATCATTC | fine deletion mapping on chromosome 5 in tla2. | | 59.9 |
| | CTTACAGCACAAA | | | |
| | TAAAGACTGAG | | | |
| K128 | GCTTTCTACCGCC | testing for co-segregation of the insertion with the tla2 | | 59.3 |
| | TGTCTCAATACC | phenotype; on chromosome 5, next to insertion, if used | | |
| | | with HK126 will only generate a product if insertion is | | |
| | | present, expected size: 1.1 kb | | |
| K126 | GGAACCCCTATTT | testing for c-segregation of the insertion with the tla2 | | 50.6 |
| | GTTTATTTTTC | phenotype; in pJD67 between bla and ARG7. If used with | | |
| | | HK128 will only generate a product if insertion is | | |
| | | present, expected size 1.1 kb | | |
| K134 | GCAGCCTTGCCAA | positive control for co-segregation PCR analysis. on | | 60.8 |
| | TCCCAAATAAGG | chromosome 5 downstream of insertion, is present in tla2 | | |
| | | and WT, if used with HK135 expected size of 0.5 kb | | |
| K135 | CTCGCACATGTCA | poisitive control for co-segregation PCR analysis. on | | 59.4 |
| | CATTAGGAGGTTC | chromosome 5 downstream of insertion, is present in tla2 | | |
| | | and WT | | |
| K289 | GATGCGGTCCACG | outer 5′ RACE for CpFTSY | | 59.5 |
| | ATCTTCAGAG | | | |
| K290 | GAGATGAGCACCT | inner 5′ RACE for CpFTSY | | 61.0 |
| | CCTCCAGCTC | | | |
| K297 | CTGTGGTGCTGAT | outer 3′ RACE for CpFTSY | | 61.6 |
| | TGTGGGAGTGAAC | | | |
| K298 | CAAGATCGCGTAC | inner 3′ RACE for CpFTSY | | 59.8 |
| | AAGTACGGCAAG | | | |
| K345 | GGAATTCCATATG | cloning of CpFTSY cDNA into pSL18 | NdeI | 68.8 |
| | CAGACGACCGTGG | | | |
| | GGCGCAAGTG | | | |
| K346 | GGAATTCCGTTCA | cloning of CpFTSY cDNA into pSL18 | EcoRI | 65.1 |
| | TTTACTTGGTGCC | | | |
| | GGCAGTGG | | | |
| ET28- | GGGAATTCCATAT | cloning CpFTSY cDNA into pET28 | deI | 0.7 |
| ftsY_up | GGCCAACGCGGGC | | | |
| | GGC | | | |
| ET28- | CCGGAATTCTTAC | cloning CpFTSY cDNA into pET28 | coRI | 0.2 |
| ftsY_down | TTGGTGCCGGCAG | | | |
| | TGGC | | | |
| ET28- | GGGAATTCCATAT | cloning CpSRP54 cDNA into pET28 | deI | 9.6 |
| srp54_up | GTCGGCCATGTTC | | | |
| | GACAGCCTG | | | |
| ET28- | CCGGAATTCCTAT | cloning CpFSTP54 cDNA into pET28 | coRI | 9.1 |
| srp54_down | TTGGACGATGAGC | | | |
| | CGAAGCCG | | | |

### Genetic Crosses and Analyses

All genetic crosses and strain matings were performed according to the protocol of Harris (1989). Prior to any physiological analysis, putative truncated light-harvesting antenna (tla) mutants were crossed four times to the Chlamydomonas wild type strain 4A+ (arg2). For co-segregation analysis of the tla2 phenotype with the pJD67 insert, tla2 was crossed to AG1x3.24 (ARG7-8). Progeny were plated on TAP medium containing arginine (TAP+Arg) and also on regular TAP-only medium (−Arg). Moreover, PCR reactions were used to test for co-segregation of the tla2 phenotype with the pJD67 insert, using the HK128/HK126 (Table S1) insertion flanking sequence specific primers set and a DNA isolation control HK135/HK134 (Table SI).

### Measurements of Photosynthetic Activity

The oxygen evolution activity of the cultures was measured at 22° C. with a Clark-type oxygen electrode illuminated with light from a halogen lamp projector. A Corning 3-69 filter (510 nm cut-off filter) defined the yellow actinic excitation via which photosynthesis measurements were made. Samples of 5 ml cell suspension contained 1.3 μM Chl were loaded into the oxygen electrode chamber. Sodium bicarbonate (100 μl of 0.5 M solution, pH 7.4) was added to the cell suspension prior to the oxygen evolution measurements to ensure that oxygen evolution was not limited by the carbon supply available to the cells. After registration of the rate of dark respiration by the cells, samples were illuminated with gradually increasing light intensities. The rate of oxygen exchange (uptake or evolution) under each of these irradiance conditions was recorded continuously for a period of about 5 min.

### Isolation of Thylakoid Membranes

Cells were harvested by centrifugation at 1,000 g for 3 min at 4° C., the pellet was stored frozen at −80° C. until all samples were ready for processing. Samples were thawed on ice and resuspended with ice-cold sonication buffer containing 50 mM Tricine (pH 7.8), 10 mM NaCl. 5 mM MgCl₂, 0.2% polyvinylpyrrolidone 40, 0.2% sodium ascorbate. 1 mM aminocaproic acid, 1 mM aminobenzamidine and 100 μM phenylmethylsulfonyl fluoride (PMSF). Cells were broken by sonication in a Branson 250 Cell Disrupter operated at 4° C. three times for 30 s each time (pulse mode, 50% duty cycle, output power 5) with 30 s cooling intervals on ice. Unbroken cells and starch grains were removed by centrifugation at 3,000 g for 4 min at 4° C. Thylakoid membranes were collected by centrifugation of the first supernatant at 75,000 g for 30 min at 4° C. The thylakoid membrane pellet was resuspended in a buffer containing 50 mM Tricine (pH 7.8), 10 mM NaCl, 5 mM MgCl₂ for spectrophotometric measurements, or 250 mM Tris-HCl (pH 6.8), 20% glycerol, 7% SDS and 2 M urea for protein analysis.

### Spectrophotometric and Kinetic Analyses

The concentration of the photosystems in thylakoid membranes was measured spectrophotometrically from the amplitude of the light-minus-dark absorbance difference signal at 700 nm (P700) for PSI, and 320 nm (Q_{A}) for PSII (Melis and Brown, 1980; Melis, 1989; Smith et al., 1990). The functional light-harvesting Chl antenna size of PSI and PSII was measured from the kinetics of P700 photo-oxidation and Q_{A} photoreduction, respectively (Melis, 1989).

### 5′ and 3′ RACE Analysis

Total RNA was isolated from CC503 cells in the early log phase of growth (0.5×10⁶ cells ml⁻¹) using the Trizol reagent (Invitrogen, USA). Genomic DNA in these samples was digested according to the protocol provided by the Turbo DNA-free kit (Ambion, USA). The RNA sample was used immediately for the 5′ and 3′ RACE analysis using the FirstChoice RLM-RACE kit (Ambio, USA) and with suitable primers (HK297/-HK298 outer/inner for 3′ RACE; HK289/HK290 outer/inner for 5′ RACE; Table SI). The manufacture's protocol was followed in all procedures.

Transformation of Chlamydomonas reinhardtii

Complementation of the tla2 strain was achieved by co-transformation of the mutant with BAC clones 08N24 and 36L15 and pBC1 plasmid (conferring paromomycin resistance) using the highly efficient electroporation method (Shimogawara et al., 1998). pBC1 contains a paromomycin resistance gene (selectable marker) operated under the control of the C. reinhardtii Hsp70A and RbcS2 promoters (Sizova et al., 2001). Further, CpFTSY cDNA was cloned into pSL18 and incorporated into the genomic DNA of the tla2 mutant using the conventional glass bead transformation protocol (Kindle, 1990). pSL18 also contains a paromomycin resistance gene (selectable marker) operated under the control of the C. reinhardtii Hsp70A and RbcS2 promoters (Sizova et al., 2001) and linked to the PsaD promoter and terminator that was used to express the CpFTSY gene. Transformants were isolated upon screening independent cell lines on the basis of the measured the Chl a/Chl b ratio of the cells.

### H6-CpFTSY and H6-CpSRP54 Protein Expression and Purification

Standard procedures were employed for isolation of plasmid DNA, restriction analysis, PCR amplification, ligation and transformation (Sambrook et al. 1989). Plasmid DNA was prepared with a plasmid purification kit supplied by Qiagen (USA). Restriction enzymes were purchased from New England Biolabs (USA). They were used according to the recommendation of the vendors. Oligonucleotides were purchased from Bioneer (USA) and sequence details are given in Table S1.

E. coli Rosetta (DE3) cells were transformed with plasmid pET28-H₆FtsY and pET28-H₆-SRP54 and grown in 1 L of LB medium in 2 L Fernbach flasks on a rotary shaker at 37° C. to a density of about OD₆₀₀=0.8. Protein expression was induced by the addition of 0.2 mM isopropyl β-D-thiogalacto-pyranoside (IPTG), and growth was continued for 4 h at 37° C. Cells were harvested by centrifugation at 4,500 g for 10 min at 4° C. Cells were resuspended in 10 ml of buffer I (50 mM Tris/HCl, pH 8.0, 400 mM NaCl. 10 mM b-ME) and lysed in a French pressure cell operated at 1,000 psi. To remove cell debris, the cell lysate was centrifuged at 13,000 g for 10 min at 4° C. The supernatant was mixed with 4 ml of Ni²⁺-NTA agarose resin (Qiagen, USA), equilibrated with buffer I, and incubated for 1 h at 4° C. The slurry was poured into a column, the flow-through was discarded and the slurry washed at 4° C. with buffer I⁵ (buffer I supplemented with 5 mM imidazole) and buffer I²⁵ (buffer I supplemented with 25 mM imidazole). H₆-FtsY was eluted with buffer I²⁰⁰ (buffer I supplemented with 200 mM imidazole). Protein fractions were analyzed by SDS-PAGE and fractions containing H₆-FtsY were pooled. The purified protein was concentrated with Amicon Ultra 15, 30 kD cut-off devices (Millipore, USA) to a final volume of 3 ml (2-3 mg/ml). The concentrate was centrifuged at 15,000 g for 5 min to remove precipitated protein. The resulting proteins were pure as judged by SDS-PAGE analysis and migrated to the expected molecular mass of about 39 kD for the CpFTSY and 54 kD for the CpSRP54 proteins, respectively (results not shown).

### Analysis of Genomic DNA Flanking the Plasmid Insert Site

Chlamydomonas reinhardtii genomic DNA flanking the plasmid insertion site was amplified using a TAIL-PCR protocol (Liu et al., 1995), optimized for Chlamydomonas genomic DNA, as recently described (Chen et al., 2003; Dent et al. 2005). Primers used for the TAIL-PCR are listed in Table S1. Briefly, flanking genomic DNA was amplified by PCR from the region adjacent to the inserted pJD67 plasmid that was used for DNA insertional mutagenesis. Specific primers for primary, secondary, and tertiary reactions were designed (Table S1). Two arbitrary degenerate primers were tested for amplification, HK030 and HK031, as previously described (Dent et al., 2005). The general TAIL-PCR protocol of Liu et al. (1995) was used with minor modifications for the various PCR amplification reactions. Nucleotide sequences of the resulting PCR products were obtained via an ABI3100 sequence analyzer. Chlamydomonas genomic DNA sequence information was obtained from the Chlamydomonas Genome Project website http:// followed by genome.jgi-psf.org/Chlre4/Chlre4.home.html and/or www site phytozome.net/chlamy/.

Chlamydomonas reinhardtii Cell Fractionation and Localization of the CpFTSY Protein

Chlamydomonas reinhardtii strain CC-503 (cw92 mt+) was cultured photo-heterotrophically in Tris-Acetate-Phosphate (TAP) medium (Harris, 1989) upon illumination of 30 μmol photons m⁻² s⁻¹ at 25° C. Cultures were grown to the early logarithmic phase with a maximum optical density of OD₇₅₀=0.5 (˜6×10⁶ cells ml⁻¹) in 250 ml Erlenmeyer flasks and harvested by centrifugation at 1,500 g for 5 min in a swinging bucket rotor (Eppendorf 5810 R centrifuge) prior to cell fractionation approaches. Cells were resuspended in cell lysis buffer (20 mM Hepes-KOH pH 7.5, 5 mM MgCl₂, 5 mM β-mercaptoethanol and 1 mM PMSF) at 4° C. and broken in a French press chamber (Aminco, USA) at 1500 psi. Total supernatant and total membrane were separated by centrifugation at 17,900 g for 30 min at 4° C. Total membranes were washed twice and resuspended to a final chlorophyll concentration of 1 mg/ml with thylakoid membrane buffer (20 mM Hepes-KOH pH 7.5, 300 mM sorbitol. 5 mM MgCl₂, 2.5 mM EDTA. 10 mM KCl and 1 mM PMSF). Total cell pellets were resuspended to 1 mg Chl per mL with 1 volume of lysis buffer and 1 volume of 2× denaturing cell extraction buffer (0.2 M Tris, pH 6.8, 4% SDS, 2 M Urea, 1 mM EDA and 20% glycerol). In addition, all denaturing samples were supplemented with a 5% (v/v) of β-mercaptoethanol and centrifuged at 17,900 g for 5 min prior to gel loading. Chloroplast enriched fractions were isolated from synchronized cultures with 12 h light/dark cycles of cell wall-deficient strain CC-503 (cw92 mt+) as in Zerges and Rochaix (1998). Western blot analyses were performed with total protein from cell extracts, resolved in precast SDS-PAGE Any KDTM (BIO-RAD, USA). Loading of samples was based on equal protein. Proteins were quantified with colorimetric Lowry-based Dc protein assay (BIO RAD, USA) and transferred to a polyvinylidene difluoride (PVDF) membrane (Immobilon-FL 0.45 μm, Millipore. USA) by a tank transfer system. Polyclonal antibodies cross-reacting with specific proteins were visualized by Supersignal West Pico Chemiluminiscent substrate detection system (Thermo Scientific, USA).

### Non-Denaturing Deriphat-PAGE

Non-denaturing Deriphat-PAGE was performed following the method developed by Peter and Thornber (1991) with the following modifications; continuous native resolving PAGE gradients (4 to 15% final concentration of acrylamide) with no stacking gel were prepared. Isolated thylakoid membranes, from wild type, tla2 mutant and tla2-complemented lines C1, C2, C3, and C4 were prepared with thylakoid membrane buffer and solubilized at a Chl concentration of 2, 1 and 0.4 mg/ml, respectively, with an equal volume of surfactant 10% n-Dodecyl-β-D-Maltoside (SIGMA). Thus, a 50:1 weight ratio of surfactant to Chl was used for the wild type. Thylakoid membranes were incubated on ice for 30 min and centrifuged at 17,900 g for 10 min in order to precipitate unsolubilized material. The amounts loaded per lane correspond to 10 μl of solubilized samples. Non-denaturing deriphat-PAGE was run for 2 h in the cold room at 5 mA constant current.

One-dimension non-denaturing deriphat PAGE strips were solubilized in the presence of Laemmli denaturing buffer (Laemmli et al. 1970) for 15 min and resolved in a denaturing 2 M Urea 12% SDS-PAGE second dimension. Acrylamide gels were stained with Coomassie (Fast Gel™ Blue R, GE Healthcare, USA) or silver nitrate gel staining according to Wray et al. (1981).

The above examples are provided by way of illustration only and not by way of limitation. Those of skill in the art will readily recognize a variety of noncritical parameters that could be changed or modified to yield essentially similar results.

All publications, accession numbers, and patent applications cited in this specification are herein incorporated by reference as if each individual publication or patent application were specifically and individually indicated to be incorporated by reference.

### REFERENCES

Aldridge C, Cain P, Robinson C (2009) Protein transport in organelles: Protein transport into and across the thylakoid membrane. FEBS J 276: 1177-86
Amin P. Sy D A C. Pilgrim M L, Parry D H, Nussaume L. Hoffman N E (1999) Arabidopsis Mutants Lacking the 43- and 54-Kilodalton Subunits of the Chloroplast Signal Recognition Particle Have Distinct Phenotypes. Plant Physiol. 121: 61-70
Arnon D (1949) Copper enzymes in isolated chloroplasts. Polyphenol oxidase in Beta vulgaris. Plant Physiol 24: 1-15
Asakura Y, Hirohashi T, Kikuchi S, Belcher S, Osborne E, Yano S, Terashima I. Barkan A, Nakai M (2004) Maize mutants lacking chloroplast FtsY exhibit pleiotropic defects in the biogenesis of thylakoid membranes. Plant Cell 16: 201-214
Asakura Y, Kikuchi S, Nakai M (2008) Non-identical contributions of two membrane-bound cpSRP components, cpFtsY and Alb3, to thylakoid biogenesis. Plant J. 56: 1007-1017
Ballottari M, Dall'Osto L. Morosinotto T. Bassi R (2007) Contrasting behavior of higher plant photosystem I and II antenna systems during acclimation. J. Biol. Chem. 282: 8947-8958
Bellafiore S, Ferris P. Naver H. Göhre V, Rochaix J D (2002) Loss of Albino3 leads to the specific depletion of the light-harvesting system. Plant Cell 14: 2303-2314
Bals T, Dünschede B, Funke S, Schünemann D (2010) Interplay between the cpSRP pathway components, the substrate LHCP and the translocase Alb3: an in vivo and in vitro study. Febs Lett. 584: 4138-41441
Chen H C, Yokthongwattana K, Newton A J. Melis A (2003) SuIP, a nuclear gene encoding a putative chloroplast-targeted sulfate permease in Chlamydomonas reinhardtii. Planta 218: 98-106
Dauvillée D, Delhaye S, Gruyer S, Slomianny C, Moretz S E, d'Hulst C, Long C A, Ball S G, Tomavo S (2010) Engineering the chloroplast targeted malarial vaccine antigens in Chlamydomonas starch granules. PLoS One 5: e15424
Davies J P, Yildiz F, Grossman A R (1994) Mutants of Chlamydomonas with aberrant responses to sulfur deprivation. Plant Cell 6: 53-63
Davies J P, Yildiz F, Grossman A R (1996) Sac1, a putative regulator that is critical for survival of Chlamydomonas reinhardtii during sulfur deprivation. EMBO J. 15: 2150-2159
Debuchy R, Purton S, Rochaix J D (1989) The arginosuccinate lyase gene of Chlamydomonas reinhardtii: an important tool for nuclear transformation and for correlating the genetic and molecular maps of the ARG7 locus. EMBO J 8: 2803-2809
Dent R M, Haglund C M, Chin B L, Kobayashi M C, Niyogi K K (2005) Functional genomics of eukaryotic photosynthesis using insertional mutagenesis of Chlamydomonas reinhardtii. Plant Physiol. 137: 545-56
Falk S, Sinning I (2010) cpSRP43 Is a Novel Chaperone Specific for Light-harvesting Chlorophyll a,b-binding Proteins. J Biol Chem. 285: 21655-21661
Glick R E. Melis A (1988) Minimum photosynthetic unit size in system-I and system-II of barley chloroplasts. Biochim Biophys Acta 934: 151-155
Gorman D S. Levine R P (1965) Cytochrome f and plastocyanin: their sequence in the photosynthetic electron transport chain of Chlamydomonas reinhardtii. Proc. Natl. Acad. Sci. USA 54: 1665-1669
Greene B A. Staehelin L A, Melis A (1988) Compensatory alterations in the photochemical apparatus of a photoregulatory, chlorophyll b-deficient mutant of maize. Plant Physiol. 87: 365-370
Greenwell H C, Laurens L M L, Shields R J, Lovitt R W, Flynn K J (2010) Placing microalgae on the biofuels priority list: a review of the technological challenges. J. R. Soc. Interface 7: 703-726
Hankamer B, Lehr F, Rupprecht J, Mussgnug J H, Posten C, Kruse O (2007) Photosynthetic biomass and H2 production by green algae: from bioengineering to bioreactor scale-up. Physiol Plant. 131: 10-21
Harris E H (1989) The Chlamydomonas source book: a comprehensive guide to biology and laboratory use. Academic Press, San Diego
Hu Q, Sommerfeld M. Jarvis E, Ghirardi M, Posewitz M, Seibert M, Darzins A (2008) Microalgal triacylglycerols as feedstocks for biofuel production: perspectives and advances. Plant J. 54: 621-39
Huesemann M H, Hausmann T S, Bartha R, Aksoy M, Weissman J C, Benemann J R (2009) Biomass productivities in wild type and pigment mutant of Cyclotella sp. (Diatom). Appl. Biochem. Biotechnol. 157: 507-526
Jaru-Ampornpan P, Shen K, Lam V Q, Ali M, Doniach S, Jia T Z, Shan S O (2010) ATP-independent reversal of a membrane protein aggregate by a chloroplast SRP subunit. Nat Struct Mol Biol. 17: 696-702
Kindle K L (1990). High-frequency nuclear transformation of Chlamydomonas reinhardtii. Proc. Natl. Acad. Sci. USA 87: 1228-1232.
Kirk J T O (1994) Light and photosynthesis in aquatic ecosystems. 2nd edn. Cambridge University Press, Cambridge
Klimyuk V I, Persello-Carticaux F, Havaux M, Contard-David P, Schuenemann D, Meiherhoff K, Gouet P, Jones J D, Hoffman N E, Nussaume L (1999) A chromodomain protein encoded by the Arabidopsis CAO gene is a plant-specific component of the chloroplast signal recognition particle pathway that is involved in LHCP targeting. Plant Cell 11: 87-99
Laemmli U K (1970) Cleavage of structural proteins during the assembly of the head of bacteriophage T4. Nature 227: 680-685
Ley A C. Mauzerall D C (1982) Absolute absorption cross sections for the photosystem II and the minimum quantum requirement for photosynthesis in Chlorella vulgaris. Biochim. Biophys. Acta 680: 95-106
Liu Y G. Mitsukawa N, Oosumi T, Whittier R F (1995) Efficient isolation and mapping of Arabidopsis thaliana T-DNA insert junctions by thermal asymmetric interlaced PCR. Plant J. 8: 457-463
Luirink J, Sinning I (2004) SRP-mediated protein targeting: structure and function revisited, BBA Mol. Cell. Res. 1694: 17-35
Mata T M, Martins A A, Caetano N S (2010) Microalgae for biodiesel production and other applications: a review. Renew Sust Energy Rev 14: 217-232
Mayfield S P, Manuell A L, Chen S, Wu J, Tran M, Siefker D, Muto M, Marin-Navarro J (2007) Chlamydomonas reinhardtii chloroplasts as protein factories. Curr. Opin Biotech. 18: 126-33
Melis A (1989) Spectroscopic methods in photosynthesis: photosystem stoichiometry and chlorophyll antenna size. Phil Trans R Soc London B 323: 397-409
Melis A (1991) Dynamics of photosynthetic membrane composition and function Biochim. Biophys. Acta 1058: 87-106
Melis A (2007) Photosynthetic H2 metabolism in Chlamydomonas reinhardtii (unicellular green algae). Planta 226: 1075-1086
Melis A (2009) Solar energy conversion efficiencies in photosynthesis: minimizing the chlorophyll antennae to maximize efficiency. Plant Science 177: 272-280
Melis A, Brown J S (1980) Stoichiometry of system I and system II in different photosynthetic membranes. Proc. Natl. Acad. Sci. USA 77: 4712-4716
Melis A, Anderson J M (1983) Structural and functional organization of the photosystems in spinach chloroplasts: Antenna size, relative electron transport capacity, and chlorophyll composition. Biochim. Biophys. Acta 724: 473-484
Melis A. Neidhardt J, Benemann J R (1999) Dunaliella salina (Chlorophyta) with small chlorophyll antenna sizes exhibit higher photosynthetic productivities and photon use efficiencies than normally pigmented cells. J. Appl. Phycol. 10: 515-525
Melis A, Spangfort M, Andersson B (1987) Light-absorption and electron transport balance between photosystem-II and photosystem-I in spinach chloroplasts. Photochem Photobiol 45: 129-136
Merchant S S, Prochnik S E, Vallon O, Harris E H, Karpowicz S J, Witman G B, Terry A, Salamov A, Fritz-Laylin L K, Maréchal-Drouard L, et al. (2007) The Chlamydomonas genome reveals the evolution of key animal and plant functions. Science 318: 245-250
Michelet L, Lefebvre-Legendre L, Burr S E, Rochaix J D, Goldschmidt-Clermont M. (2011)

Enhanced chloroplast transgene expression in a nuclear mutant of Chlamydomonas. Plant Biotechnol J. 9: 565-574
Mitra M. Melis A (2010) Genetic and biochemical analysis of the TLA1 gene in Chlamydomonas reinhardtii. Planta 231:729-740
Mussgnug J H, Wobbe L, Elles I, Claus C, Hamilton M, Fink A, Kahmann U, Kapazoglou A, Mullineaux C W. Hippler M, Nickelsen J, Nixon P J, Kruse O (2005) NAB1 Is an RNA Binding Protein Involved in the Light-Regulated Differential Expression of the Light-Harvesting Antenna of Chlamydomonas reinhardtii. Plant Cell 17: 3409-3421
Nakajima Y, Ueda R (1997) Improvement of photosynthesis in dense microalgal suspension by reduction of light harvesting pigments. J. Appl. Phycol. 9: 503-510
Nakajima Y, Ueda R (1999) Improvement of microalgal photosynthetic productivity by reducing the content of light harvesting pigments. J. Appl. Phycol. 11: 195-201
Peter G F, Thornber P J (1991) Biochemical composition and organization of higher plant photosystem 11 light-harvesting pigment proteins, J. Biol. Chem. 266: 16745-16754
Pilgrim M L, van Wijk K J. Parry D H, Sy D A, Hoffman N E (1998) Expression of a dominant negative form of cpSRP54 inhibits chloroplast biogenesis in Arabidopsis. Plant J 13: 177-186
Polle J E, Benemann J R. Tanaka A. Melis A (2000) Photosynthetic apparatus organization and function in wild type and a Chl b-less mutant of Chlamydomonas reinhardtii. Dependence on carbon source. Planta 211: 335-344
Polle J E, Kanakagiri S D, Melis A (2003) tla1, a DNA insertional transformant of the green alga Chlamydomonas reinhardtii with a truncated light-harvesting chlorophyll antenna size. Planta 217: 49-59
Powles S B. Critchley C (1980) Effect of light intensity growth on photoinhibition of intact attached bean leaflets. Plant Physiol. 65: 1181-1187
Sambrook J, Fritsch E F, Maniatis T (1989) Molecular Cloning: A Laboratory Manual. Cold Spring Harbor, N.Y.: Cold Spring Harbor Laboratory Press Shimogawara K, Fujiwara S, Grossman A. Usuda H (1998) High-efficiency transformation of Chlamydomonas reinhardtii by electroporation. Genetics 148: 1821-1828
Sizova I A, Fuhrmann M, Hegemann P (2001) A Streptomyces rimosus aph VIII gene coding for a new type phosphotransferase provides stable antibiotic resistance to Chlamydomonas reinhardtii. Gene 277: 221-229.
Smith B M. Morrissey P J, Guenther J E. Nemson J A, Harrison M A. Allen J F. Melis A (1990) Response of the photosynthetic apparatus in Dunaliella salina (green algae) to irradiance stress. Plant Physiol 93: 1433-1440
Tetali S D, Mitra M, Melis A (2007) Development of the light-harvesting chlorophyll antenna in the green alga Chlamydomonas reinhardtii is regulated by the novel Tla1 gene. Planta 225: 813-829
Tu C J, Schuenemann D, Hoffman N E (1999) Chloroplast FtsY, chloroplast signal recognition particle, and GTP are required to reconstitute the soluble phase of light-harvesting chlorophyll protein transport into thylakoid membranes. J. Biol. Chem. 274: 27219-27224
Wray W, Boulikas T, Wray V P, Hancock R (1981) Silver staining of proteins in polyacrylamide gels. Anal Biochem. 118: 197-203
Yi L, Dalbey R E (2005) Oxa1/Alb3/YidC system for insertion of membrane proteins in mitochondria, chloroplasts and bacteria (review). Mol. Membr. Biol. 22: 101-111
Zerges W, Rochaix J D (1998). Low-Density membranes are associated with RNA-binding proteins and thylakoids in the chloroplast of Chlamydomonas reinhardtii. J Cell Biol. 140: 101-110
Zouni A, Witt H T, Kern J, Fromme P, Krauss N, Saenger W, Orth P (2001) Crystal structure of photosystem II from Synechococcus elongatus at 3.8 A resolution. Nature 409: 739-743

Table of Illustrative Tla2 nucleic acid and polypeptide sequences
from Chlamydomonas reinhardtii

SEQ ID NO: 1 Tla2 cDNA sequence
ATGCAGACGACCGTGGGGCGCAAGTGCGTCGCGAGCTCCGCAGCTGGGCGCAGC
CGCAATGTTACTGTCTTCCGTAGGTGCAGCAGGGGCGGCCCTGTGAAGGTGGTCG
CCAACGCGGGCGGCGAGGCCGGCCCCGGCTTCCTGCAGCGCCTAGGCCGCGTCA
TCAAGGAGAAGGCCGCCGGCGATTTTGACCGCTTCTTCGCGGGGACCTCCAAGA
CGCGCGAGCGCCTGGGGCTGGTGGATGAGATGCTGGCGTTGTGGAGCCTGGAGG
ACTACGAGGACAGCCTGGAGGAGCTGGAGGAGGTGCTCATCTCCGCCGACTTCG
GCCCCCGCACTGCTCTGAAGATCGTGGACCGCATCCGCGAGGGCGTCAAGGCCG
GCCGCGTCAAgAGCGCCGAGGACATCCGCGCCTCGCTCAAGGCTGCCATTGTGG
AGCTGCTGACGGCACGGGGGCGCTCCAGCGAGCTCAAGCTGCAGGGCCGGCCGG
CTGTGGTGCTGATTGTGGGAGTGAACGGCGCGGGCAAGACCACCACTGTGGGCA
AGATCGCGTACAAGTACGGCAAGGAGGGAGCCAAGGTCTTCCTCATCCCAGGCG
ACACCTTCCGCGCCGCCGCCGCGGAGCAGCTGGCGGAGTGGAGCCGCCGCGCGG
GCGCCACCATCGGCGCCTTCCGAGAGGGTGCCCGACCGCAGGCCGTCATCGCCTC
GAACCTGGACGACCTGCGGCAGCGCACGTGCAAGGACGCGTCGGACGTGTACGA
CCTCATTCTGGTAGACACGGCAGGCCGGCTGCACACGGCCTACAAGCTGATGGA
GGAGCTGGCGCTGTGCAAGGCGGCGGTCAGCAACGCACTGCCGGGCCAGCCCGA
CGAGACCCTACTTGTGCTAGACGGCACCACAGGCCTCAACATGCTAAACCAGGC
CAAGGAGTTCAACGAGGCTGTGCGGCTGAGCGGACTCATCCTCACAAAGCTGGA
TGGCACTGCCCGAGGAGGCGCGGTGGTGAGTGTGGTGGACCAGTTGGGCCTGCC
CGTCAAGTTCATTGGTGTGGGCGAGACGGCCGAGGACCTGCAGCCCTTCGACCCC
GAGGCATTCGCTGAGGCCCTGTTCCCGAAGGTCAAGGAGCCCGCCACTGCCGGC
ACCAAGTAA
SEQ ID NO: 2 Tla2 polypeptide sequence
MQTTVGRKCVASSAAGRSNVTVFRRCSRGGPVKVVANAGGEAGPGFLQRLGRVI
KEKAAGDFDRFFAGTSKTRERLGLVDEMLALWSLEDYEDSLEELEEVLISADFGPRT
ALKFVDRIREGVKAGRVKSAEDIRASILKAAIVELLTARGRSSELKLQGRPAVVILIVGY
NGAGKTTTVGKIAYKYGKEGAKVFLIPGDTFRAAAAEQLAEWSRRAGATIGAFREG
ARPQAVIASNLDDLRQRTCKDASLWYDLILVDTAGRLHTAYKLMEELALCKAAVSN
ALPGQPDETLVLLDGITGLNMLNQAKEFNEAVRLSGULTKLDGTARGGAVVSVVD
QLGLPVKFIGIVGETAEDLQPFDPEAFAEALFPKVKEPATAGTK
Domains:
Amino acids 1-36: Transit peptide (ChloroP)
Amino acids 66-147: Helical bundle domain (Pfam), SRP54-type-protein
Amino acids 162-370: GTPase domain (Pfam), SRP54-type protein
Amino acids 164-183: P-loop nucleotide binding motif, (pre)
SEQ ID NO: 3 Tla2 genomic sequence
GTGACAGGCCCGGCGCGCACGCCGCATACACAACGCGTCTGCCTCTGGCCGGCC
GGCCTGCCGTGTCTGCCGCTGATATGCCCTGGTACCCCCGCTAACCCAGGAGCGC
AAGCTCACCCGCCGATGIAGAAGTAGGCATTTAGGAATAGCCATGCGTAGAAGA
TGACGGTGCGGGCCCGCAGCGGCAGCAGCGGCCGCCAAGCGGATGTGCGCCAGG
ACGGACGCGTCGCTGCGTACAGCACCCCGACGCCCAGCAGCAGCAGCACATTGA
TGGCCGCTGCCAGAGGAATTGTCTCCGCCCATGGCCATGGAAGCCCCCAAACGT
GGCTTTCATCGGGGGACAGCCAGACCACCGCTGTCFCCGAGGATGGCGACCGCGA
GGAGCGCCAGGCAGGGGCGCCACCCCACCGGAACTTGAATGCGAGCCGAGTAGG
TCATGCAGCTGCGCGTGCTGGTCTCCAGGAGGCGCCGAGCATGCCGGTAGACTAT
CCTCCGCTTTGAATCAAGGTGCAATTTCAGGTCCTCAGGCTGAGCAACAGTTGCC
TGACTCGGTGGAGCTGCAGGTTGTAATGGGTATTGTCCACCTCTTCTTCTTGCCGA
GGGCACCCATGGCCCCTGCGAGGCCATGGTTTGCGGCTTCGCGAACACCTAACTT
GCGTGCATAAAGCGTGCGACAGGCACGCGCCGCTGCTGAGCTGGCTTAATTTCTA
CAGTAGTCTCACTTGTGCAACTGAATATGTAATTGCGATTAAGGAGCGAGGTTTG
TGGGCTGTTGCGCCAATGGTCACTCCGGTACAACTCCGGAACCGACACGCAGCA
GTAGCCCCCAAGATTTAGCCCCAACTTCAGCCCTCAAGTTCAGCGGGTTTGGGTC
AGAACAACGACAGGGTGTGCTGATAGCGACTTAACACTCTAAGCAGCAAGAGCT
GGCAAGGCGGCTTTCATAAGCAACAGCCAGGGCAAGCGTGTGCTCGCGCTTCAG
TCAAACTGTCAAACACCTCGTGTTGTTACGGTATCAAGGCAGAAGCGCTCTGCAA
AACGACTAGGCCTAAGTGGACGTGCGTTCTTTAAATGGTAGCAACATGGCGCTGC
ATGGCGCCCTGCCGTTTGCCCGTTTCCCGTCGTTTCCCAGGGTTGGGGCACCCCTG
CCGTATCCCACTTTCTCCCGCTCTCAAGCGATCAGCCCGGTCCCCCCTTCCACACT
GCCGCGAGCACACAGGAGAATACCTGGCAGCATATACTTGCGCAGGACACTGGG
ACTGCGCCAATGCACGAATGTGACCTGCTGCGGAGGAACCCCTGTGTCGAGTCG
ACTGTTTCAATGCCAAACCCTTGAGTTATGGTCCAAGGTCAGACAGATGGTATGA
AAGACGGTACTAACACACGGCAGTCTACGCACAATATACCTCGTTCGGACACCG
ATGACCTGCGATGTCGATGCGCTTTAAGTGTTCCTTGCATAGTGTCAAAGGACGG
GTGCGGTGCGCATGAAGAACCTATAGTGACGTCCGGGGGTGTCAGGACGGGGCG
TTTTGTAAAGTGAGGTCCAGAGGAGCGCAGGGTAGGGGGCATGTGGAAGGTCGG
GGCGTGTCGGGGGGTGGGGAGTGGGGGCACGAGTGGGGGTGGGGAACCGCATT
GGAGATCCAGGTCGGTTGCCCCGGCGAGTTTTGCCGCGCGCCACCCAAATGCGG
ACTTTAACGCTTGAGGAACACATACAAACAGTAGCGAGTTCGCGCCTGCTTTCCG
CGTCTGCACCGCACTCCACGTCGACTGCTTTTTCGTTTTCAGGCTTTGTTTTACAA
GCCGACTTATCTTACAATTAATTGGTTGTTCATAGACCCTACATTGATCCGAGGA
TCTAGCGATTGTGCACAGGAAAGCCGAAATGCAGACGACCGTGGGGCGCAAGTG
CGTCGCGAGCTCCGCAGCTGGGCGCAGCCGCAATGTTACTGTCTTCCGTAGGTGC
AGCAGGGGCGGCCCTGTGAAGGTACGCGACAGGCTCAGTGGCCGTTTGGCGATT
GCACACTCCTGTTCGTCGCGCCAGCCTGCGAACCCAGCTGTCATACTTATATGAT
AGTGTGGAAGGCCACGGTGGTTGGGTTGGCCAGCGGGGCTCACGCGGAGCGGTT
CCGCGGACACGGTCCCTGCTTCAGACAGCCCTACCCCTTCCCGCCCTCTCATGCC
CCACGCGCGCTGCTCGCGGCTGTGCAGGTGGTCGCCAACGCGGGCGGCGAGGCC
GGCCCCGGCTTCCTGCAGCGCCTAGGCCGCGTCATCAAGGAGAAGGCCGCCGGC
GATTTTGACCGCTTCTTCGCGGGGACCTCCAAGACGCGCGAGCGCCTGGGGGTAA
GTGGCGCCGTGCAGTGCCCTTCACAAGCCCGCATAGGCCGCAACACCTGCCGCCC
GACGTGCTTTGCTGCACCCGTACTTGCCGACCCAATCACCACAAATGCACACAAG
CCCGCTCCCCGTGTGCTTTCCCAACAACCAACTTGCCCGCAGCTGGTGGATGAGA
TGCTGGCGTTGTGGAGCCTGGAGGACTACGAGGACAGCCTGGAGGAGCTGGAGG
AGGTGCTCATCGTGAGTGGGGCTTGGGCTTGGGCGGGGGGTGGAAGGGGAGGGG
TTACATTCATGACTAGTGACTAGCGGAAGGGGGGTTACTTTACTCCAAAGGCAGT
GGGCTGGTGGGGATGGGGAGCGGGCGGGTGGTCGTGGTGGTTGCGGTCGTGGTG
CTGATCGTGGATGAAGGTTGGGGCAGGGAGGGTGTGTGTGTGCTGGCAGCAGTG
TGCGTGGGTGTGGGTGTGGGTGTGGGTGTGGGACTGTGGGTGTGGGTGTGGGTGT
GGGTGTGGGTGTGGGTGTGGGTGTGGGTGTGGGTGTGGGTGTAGGACTGTGGGT
GTGGGTGTGGGTGTGGGTGTGGGTGTGGGACTGTGGGTGTGGGTGTGGGTGTGG
GTGTGGGTGCGGTGCGGGTGTGGGTTTGGGTTTGGGCGTGGGTGTGGGTGTGGG
GGTTAACTCTGAACCAATCCCATAAGGAAACAGAATCGCTGCACGGGGAAGGGT
GTGAAGGAAGAGGGAGAGGGTGCCGTGGAAGACAGGCACAATGGATGTTAGGT
GTTGCCGGGGGAGTGCCAGTTTGGGAGCGGCTTTGGATGGGGTTGTTTTCACCAT
TCCCACTAAGAAACCCACAGACACGACGGGGGGAACCTAGTTCCAAAAGGGCAT
GGTCGTTGCACACAAGAACGCATGACATGGGCAGTCAAGCCCAACCCTGAATAC
AGGGGGGCAGCGGAGCAGGGGGCAGCGCGTGCTAGACCTGACGGGTACTGGGG
TCTGAACGGTCGGAGGGGACAGGGTTCGGCAAGGGCATGCACCGAGTCCCAAGC
GCCAAGGGCACGCACCTTGCACCGCAGACCCAGACAGCCGTGTGACGGGCTGAC
GGCGAGAGGCTGGCTTTCTTGGCATGACCTGCATGCTGCAGAGGGATCAAGGCC
CAAGGGCCCCGGGGGTGTCCCCGCAATCACCAACCCGCCCACCCGCCCACCCCTT
CACCCGCTCTCCTGCGTCACCTCGAATCACACACCCCCACCGCCATCCGCTTCTGT
CCACAGTCCGCCGACTTCGGCCCCCGCACTGCTCTGAAGATCGTGGACCGCATCC
GCGAGGGCGTCAAGGCCGGCCGCGTCAAGAGCGCCGAGGACATCCGCGCCTCGC
TCAAGGTGGGTGTGGGTGTGTTAGTGTTTGTGTTTGTGTGTGTATGTGTGTGTGTG
TGTGTGTGTGTGTGTGTGTGTGTGTGTGTGTGTGTGTGTGTGTGTGTGTGTGTGTC
AAGTACGAAACGAAAACCCGCCCAATAACAGCACCGGAGTTAAAGTGTGTGTGG
GGGGGGGAGGTTTAACACTGAACCAATCCCGTAAGGAAACAGTCGCTACAAGGA
GAAACTGCATCCGCATATGCGAGCTACTTGACTTCGGTTGTCGGGGTTGACTTCG
GTTGTCGGGGTTGACTTCGGTTGTCGGGGTCCGCGGGTCGTTTACTCCAATGCCT
AAGATACGGGGTTGACATGCCCGAGCCCATTAGCCAAGGAATCGTCGCAAGGGA
TTGGGGGGTTGCAGATCTCTAAATGAAGCAGGGGGGTGCAGACGGTGTTGGGAG
CACGCGTGTGCGCGTGCGTGTGCGTATCTGGTGCGCGTGGCTGGGCGGGTGGTGT
GCGTGGGGAGGGTGGTTCAATGTGTGCAGACACGCCGGTCCAGCTCAGTTACGC
GGCCGTCCTTCACCTCGCTGCCATGGTCTGTACACGCAGGCTGCCATTGTGGAGC
TGCTGACGGCACGGGGGCGCTCCAGCGAGCTCAAGCTGCAGGTGCGTCATGAGG
GAGGCGGACCGGAACTGGAGAGGAAGCCAGAAACAAACTGAGGTTCAGGAAGG
AGTGAGATGACTTGGACCGCATTCACGGCTGCTGTTAACAGCTGCTGCAGCGGA
GCCCACGCGCGAGCGAGCGGAGCTCCTCAAAAGCTAGGTCCCTGAATTGCGCCG
CACGTTGCAACACTGCAGGGCCGGCCGGCTGTGGTGCTGATTGTGGGAGTGAAC
GGCGCGGGCAAGACCACCACTGTGGGCAAGATCGCGTACAAGTACGGCAAGGA
GGGAGCCAAGGTAAGCTTGGGCTGGAGCGGGCGGAGCAGAAGAGGTGGAGGAG
GAGGGAGGAGGAGTAGCTCGGAAGGAGGGTTGGGTAGGGCCGGAGGAGGCTGT
GGGGCGACTGGGTGGGTGCGGCAGGCAGTGGTAAAGGGAGCTGCTAGGCAGGT
GGTTGTGGGACCCGTGTCGGGATGCTGTACGGGCGTGATGGTGGGGAGTTCGGT
GGGCAGTTCGGATGTATCGTAAGCGCGTGGGCACACCAGCGGTTGTCGAGGCAG
CAGCAGCTGCGGCATCACGTGACCATTGCTACAACCCTGTCAGCACACGCGTCCG
CCACGCGAATGCTATATCCTTACATGCATACAGACCGCGCGCACACTCGCCTCCA
GACTGCTGCCGTTCCGAACCGGCATACGGTATGTCGTTATCCTGACCGCGCGACC
TTGCGGGCACACGCGACCGCACGTGCGCTGCGCAGGTCTTCCTCATCCCAGGCGA
CACCTTCCGCGCCGCCGCCGCGGAGCAGCTGGCGGAGTGGAGCCGCCGCGCGGG
CGCCACCATCGGCGCCTTCCGAGAGGGTGCCCGACCGCAGGCCGTCATCGCCTCG
GTGAGTGCGGGGTGCAGTGGGCGTAAGGGGTTGTGTTTACCAGCCCAAACTTGA
GCAAGACAAAGGGAATTCAGCGGAGCACAGGGGAGGGGGCTGTGGGCACGGTA
TGGGGCATGTTGCGGGTGCGGTTAGCGGAGGGTGTGTGCTAGGGGAGCACGTGC
AGCGGTGTGCAGTGGGGCAATTTCGAATCGTATAGCATCCCTGTCCTTGCTGGTA
TGCGTCTTGCCTCTGCCGTGCCCCTCATCTGTATGACCATTACCCACTCCACCTTT
CACGGACGGCTACCCCCAACCACCACCACTATCCGTCATCGCGCCCATCGGCAAC
CACCGTACACCAACTACCACCGGCCCAACCACCATCTCCCCAACCGCCAACCAAC
AGAACCTGGACGACCTGCGGCAGCGCACGTGCAAGGACGCGTCGGACGTGTACG
ACCTCATTCTGGTAGACACGGCAGGTGCGTGTCTGGGGGCGAGGGTTGCGGGTA
GCGTGCAATAGGGGTTGCFCTAACGTTCGGTTTCCTTCCGCCTAAAGGCTTGCGT
CCGCCTTCACACATCAAACGCCACTATCCATTTCCCACCAGCACCGCCCCACCAC
CCCAACCACCGCCCCAACCACCGCCCCAACCACCGCCCCGACCACCACCCCAAC
CACCGCCCCAACCACCGCCCCAACCACCACACCACCCCAACCACCAGGCCGGCT
GCACACGGCCTACAAGGTGATGGAGGAGCTGGCGCTGTGCAAGGCGGCGGTCAG
CAACGCACTGCCGGGCCAGGTGCGGGCGGCGGACGGGGGGTTACATTCATGATA
TTTAAGAAGTGAAGTCGTAGGGGGGGCGGGCGTTGACGTTGGCGCTGTTGCGTCT
TTGCGTGGATGCGTGGCGGGGCGCCGGTGCTCCTCAAGCCGGATCTGCAGTGCCT
GAAGGCCTCGCGCTGCCAGGCTGCCATGCATGCGGTCCATGCGACCCTTGCCTGG
GCCGTCTGCCGCAACCAGCCCCGATCACAGTCCGCCCCACGCATCTATCTGTGCA
CGCCGTTAACTCATCAATTGGCAATGCCTGGTGATGCCCGCTCCTTGTTGCCGCC
CCGCGCAGCCCGACGAGACCCTACJTGTGCTAGACGGCACCACAGGCCTCAACA
TGCTAAACCAGGTGGGGCGGAGTGCAGGATGTGTGTATGTGTTATACATGCAAA
GGTGCAAAACGCAAAGGCACAGTCGGCGGGTGGTGCCGCGCCGGCCCCACTTTG
CGCCACCGCATCATATGCGCTACCCGGCGCCTTTCFGCCTGGAGTCCTGAGGTCT
AGCTGCCCAACAGGCCCTTCCCTTGTGCTCTCTAACCTCGCCCAACCGCTGCGCTT
GCAGGCCAAGGAGTTCAACGAGGCTGTGCGGCTGAGCGGACTCATCCTCACAAA
GCTGGATGGCACTGCCCGAGGAGGTGCGCATATGTGTGTGTGTGTGTCTGTGCGT
GTCTGTGCGTGTACCGTATGTGTATGCGTGTGTGCGTGTGCGTGTGCTTGTAGTCC
CACGCGTATCAATCGCCCGCCCCCGGCTTGCAAGGATACCTCTCATGACTCATGG
GGCACTGTAAATGCAACTGGTGCATAGGGAAGCACAAGGGAAAAGGGTCGATAC
CATTTAAATGCGTGGGGTTGCGGCGTTGTATTCACACTGCCCCAAGCATTCCTGA
CAACGGCAGGCAGTGACCAGTGGATTTGTGAGTGTGAGGCGCCCATTGCTGCAC
CTCCCGGTGTTGCTCCATAGCCCACCGTTCTGTGTCCGCCGGTCCTTAGTGTGTCT
ACCGCTTGATTTCTCCTTGCCATTCGCTTGGTTFGGTTAGTTTAGGTTTGGGCTGG
TACCCAAGCTACCCCTGCCCCCCTGCTGTTCCGTGTCTCTCACCCCGTGTCGCTGT
CCCCCTCCAGGCGCGGTGGTGAGTGTGGTGGACCAGTTGGGCCTGCCCGTCAAGT
TCATTGGTGTGGGCGAGACGGCCGAGGACCTGCAGCCCTTCGACCCCGAGGTGG
GCAGCAGCAGACCGGGGGGGCGGGGGGTGGGGGGGCCAGCCGTGGCTCGAATG
GGGGAGAAGCGGAGAGTGGGGGAGGCGGCACGGGGAGGCGGTGGGGAGGGTTA
CGGGGATCAAACACATACCGTAGGAGCGCTGGGCTGCACTGCAATGCCCCTGCG
TCCCCGCGGGGGCGAGGCTTACGGCAGTTGGGTTGCAGTCATGAACGGGGCATT
CTGCGTGCCTGGTTTGCAGTAGCGAGCGGGGGGTGACGGCGTTGGCGTTGGCGT
GCGCAAATGCACACACGCTTTGCAAATTCCACACCACCGCTCTGCACGTCCGGAA
CGACGCAGCGTCCGGCGCCCCGCTCCATCCCCCCCTTTGGGGTTGGAACAAACTA
CTCCCTAAAATACAAGTACAATGCCGCCTTGCCCCTTCTATCCCCCCGACGCACC
CAGCCGACTTTCCCTATCCGATGCCGGTGCCTGTCCTGCCTGTCGCTACACAGGC
ATTCGCTGAGGCCCTGTTCCCGAAGGTCAAGGAGCCCGCCACTGCCGGCACCAA
GTAAATGAACGGGGCGGAGCACTGCTGGGGGCGCCTGGGGGCAAATACGTTTGG
GCATGCTGGTATGGATGTGGGCTTACTTGTTTTCTAAGTGGAGGTGGGCTTTGGG
ATGCAGGTGTGTGAAGTCTGTACTGCGTTTCGTGTTGGTTTGTGCGAGAGAGCGC
GATGAGTGACTCGTGGTGTGTCGCCATTTGCAAGGAATTTCAAACCTGCGTGAGG
GAAGGCAACAGGGGTTGCGGTCACTGGGTGCGCCCCTGCAAGGCCAAAGTTGAT
AGGATGGCGCGATTGGCGCCTAAAGCGTTTATACTGCTCGCGGCACAGAACTGG
GCGGGTACGCCTTCGCAATGTATGGAGGTGCAGTTTGTGGATGCCGGTCTCTTTG
GCGCGCCGCACTCTTGTGGAAGTGGGTCGCCGCGGTGTGGCGAACGGCCTAAGG
CGGCAACGGGCAACGCGTGTGAACGAGGGTGTGCGACGGGACAACGGTGGCAA
GGCAGGTTGGGAAGTCCCGTAGGATGCAGCTGATTCCCAGAACCCAGCAGATGG
CAAACAATTTGCAATGGGCAACGCGTGTGAACGAGGGGAGCACGGAGCACCCTG
GCGGGCACCCGGCACGCCAGCTGTGCAAGCAGTGGTACTGGCCTAGGTGGACGA
GGGCGGCCTGCCCTTGTCGGCCACTCAGCCCGGTACTTGCAGTGGAAGCGGAGG
AGGGATGCGAGCGGCCACCTAGGCAATCAGCGTGGCGGCGGACTAGCGGCGGCC
TCCTGGGGGCGGAGGAGTGCAAAGGAACGTGTAAATACGGAGGAAGCGGAGGG
AAGGAAGCAATAGGAGGTGTGGCTGTCGTAGGCTCGGATGGAATGGTGGAGCAA
GGCAGAGCACGTAAGGCGGAGCATGACTGGGACAAGCAGGCAGCACAGCCGTT
GTGCAAGCACTCCGTCTTCATTTGTGCTGTAAGGGATGATAAGGTGCATATGAGG
TTTCTGGCCCCCGGCGCGGTGGGTGTCGTGTGGCAAGTGGCAAGGGAGCTTCGCG
TGTTGGGACGTGAAGTCCGGTGGGGGCGCGAGCCCATGTGCCCATCCAGCGAAG
CCCTAGCGCCTGCGTCTGCGCGCATGTGAAGGCGGGGGCAAGCGCTGTCAGAGC
GCCAGGTGGATGTGGGCCTTGTGGGCGGAACAGCGGGGGGCGGCAGGTGTAGGT
GGCGAGGGCATCGGCTCAAGAGCCTCGGGAGTGCCTCGGGGGCCCGGGGGCCCA
AGCGGCAGCATGATGCGAGTGGCGCACGCATTGTGTGCATTCCGCATCCGGTCA
AGCGCGGCACATTCCATGCTGTTGTGCTTTCACTACTTGCGCAACTGCTACATGC
CGACACACAAATCTGTACTCGGTGCAGTTCACTGTGGCTTGGCTCCCATTCGCTTT
GACCGCAATAACCTACTTTGCCGCCGAGCGAGCGGCTGGCAGCGAGTGTTTGAT
ACCTTGCGCACCTCTAAGTGTTATTTAGGCGATAGCAAGCTTCAGTCACGTCACT
GCACGTGCTCATTTCTCGGTTCTTGGGAGTGCATGCCCTGGTTTCGGCCCCGACTA
GTGCCGCGCGCCCCTGCGTGTGAGCACAAATACCTTTGCCCAGCATATCATATGT
TTGCTATGAACGGCAGCAGTCTACATCGACGACTGTCGGAGCGTGTCGGAGCGT
GAACCCGGCTGCACGCCGACGTGGCTCGCTTCAATCCTTGGGGGACGCCCTGCGC
CCTTCACCTGCCCACGTAAGCTTTGCTGCGCGAGTCATGCAATAGCACATAATGA
GTAGTGCCATTGCATTGCTGTAGTACCGTGCGAGTCGGGCTGCAAGCGGTTGGGC
GCGTACCTACCCGTGTATGGCTGCGGCCGCGCTAAATGTGCCCCGTGGGTGGGCG
AGCTCTATATCGCGCTGCACGGGTTGACAGCACAGCAATAGATTGGCCGCCCAG
CTTGCATAGCTCGATGGTGTACGCGCCGGGCGGCACGTTGTCAAGCGCCGCCAG
GATGGAGGGCGGCAGCTTCGGCFGGCFGCTGAATCGCTGAATCTCCFCCAGCGCG
TCAGAGTCCAGCT

1. A method of decreasing chlorophyll antenna size in a strain of green microalgae, the method comprising:
inhibiting expression of a Tla2 nucleic acid in the green algae strain by introducing into the green microalgae strain an expression cassette comprising a promoter operably linked to a polynucleotide that suppresses expression of Tla2; and
selecting algae with decreased chlorophyll antenna size compared to green algae in which the expression cassette has not been introduced.

2. The method of claim 1, wherein the polynucleotide is at least 90% identical to at least 200 contiguous nucleotides of a nucleic acid sequence encoding SEQ ID NO:2; or comprises at least 20 contiguous nucleotides of SEQ ID NO:1 or SEQ ID NO:3.

3. The method of claim 1, wherein the promoter is constitutive.

4. The method of claim 1, wherein the promoter is inducible.

5. The method of claim 1, wherein the polynucleotide is operably linked to the promoter in the antisense orientation.

6. The method of claim 1, wherein the polynucleotide is operably linked to the promoter in the sense orientation.

7. The method of claim 1, wherein the polynucleotide is an siRNA.

8. The method of claim 1, wherein the polynucleotide comprises at least 200 contiguous nucleotides of a nucleic acid that encodes SEQ ID NO:2.

9. The method of claim 1, wherein the nucleic acid that encodes SEQ ID NO:2 has the sequence set forth in SEQ ID NO:3 or SEQ ID NO:1.

10. The method of claim 1, wherein the green microalgae is Chlamydomonas reinhardtii, Scenedesmus obliquus, Nannochloropsis, Chlorella, Botryococcus braunii, Botryococcus sudeticus, Dunaliella salina, or Haematococcus pluvialis.

11. The method of claim 1, wherein the green microalgae comprises a heterologous isoprene synthase gene operably linked to a promoter.

12. A strain of green microalgae comprising an expression cassette comprising a polynucleotide, or a complement thereof, that is at least 90% percent identical to at least 200 contiguous nucleotides of a sequence encoding SEQ ID NO:2; or comprises at least 20 contiguous nucleotides to SEQ ID NO:1 or SEQ ID NO:3, or the complement thereof.

13. The strain of green microalgae of claim 12, wherein the green microalgae is selected from Chlamydomonas reinhardtii, Scenedesmus obliquus, Nannochloropsis, Chlorella, Botryococcus braunii, Botryococcus sudeticus, Dunaliella salina, or Haematococcus pluvialis.

14. The strain of green microalgae of claim 12, wherein the green microalgae comprise a heterologous isoprene synthase gene operably linked to a promoter.

15. A method of enhancing yields of photosynthetic productivity under high cell-density growth conditions, the method comprising cultivating the strain of green microalgae of claim 12 under bright sunlight and high cell density growth conditions.

16. (canceled)

17. A method of enhancing isoprene production, the method comprising cultivating the strain of green microalgae of claim 14 under bright sunlight and high cell density growth conditions.

18. A method of enhancing bio-oil or bio-diesel production, the method comprising cultivating the strain of green microalgae of claim 12 under conditions in which bio-oil or bio-diesel is produced.

19. (canceled)

20. A method of enhancing Beta-carotene, lutein or zeaxanthin production, the method comprising cultivating the green microalgae of claim 12 under conditions in which Beta-carotene, lutein or zeaxanthin is produced.

21. (canceled)

22. A method of enhancing astaxanthin production, the method comprising cultivating the green microalgae of claim 12 under conditions in which astaxanthin is produced.

23. (canceled)

24. A method of screening for green algae that show enhanced yield of photosynthetic productivity under high-density growth conditions, the method comprising:
introducing mutations into a population of green algae; and
screening the green algae for inhibition of Tla2 gene expression, wherein inhibition of Tla2 gene expression is determined by measuring the level of Tla2 mRNA or Tla2 protein.

25.-26. (canceled)


SEQUENCE LISTING (62 sequences)

SEQ ID NO: 1 - DNA, 1146 bp, Chlamydomonas reinhardtii
  Signal Recognition Particle (SRP) component Tla2, CpFTSY cDNA
atgcagacgaccgtggggcgcaagtgcgtcgcgagctccgcagctgggcgcagccgcaatgttactgtcttccgtaggtgcagcaggggcggccctgtgaaggtggtcgccaacgcgggcggcgaggccggccccggcttcctgcagcgcctaggccgcgtcatcaaggagaaggccgccggcgattttgaccgcttcttcgcggggacctccaagacgcgcgagcgcctggggctggtggatgagatgctggcgttgtggagcctggaggactacgaggacagcctggaggagctggaggaggtgctcatctccgccgacttcggcccccgcactgctctgaagatcgtggaccgcatccgcgagggcgtcaaggccggccgcgtcaagagcgccgaggacatccgcgcctcgctcaaggctgccattgtggagctgctgacggcacgggggcgctccagcgagctcaagctgcagggccggccggctgtggtgctgattgtgggagtgaacggcgcgggcaagaccaccactgtgggcaagatcgcgtacaagtacggcaaggagggagccaaggtcttcctcatcccaggcgacaccttccgcgccgccgccgcggagcagctggcggagtggagccgccgcgcgggcgccaccatcggcgccttccgagagggtgcccgaccgcaggccgtcatcgcctcgaacctggacgacctgcggcagcgcacgtgcaaggacgcgtcggacgtgtacgacctcattctggtagacacggcaggccggctgcacacggcctacaagctgatggaggagctggcgctgtgcaaggcggcggtcagcaacgcactgccgggccagcccgacgagaccctacttgtgctagacggcaccacaggcctcaacatgctaaaccaggccaaggagttcaacgaggctgtgcggctgagcggactcatcctcacaaagctggatggcactgcccgaggaggcgcggtggtgagtgtggtggaccagttgggcctgcccgtcaagttcattggtgtgggcgagacggccgaggacctgcagcccttcgaccccgaggcattcgctgaggccctgttcccgaaggtcaaggagcccgccactgccggcaccaagtaa

SEQ ID NO: 2 - protein, 381 aa, Chlamydomonas reinhardtii
  Signal Recognition Particle (SRP) component Tla2, CpFTSY
MQTTVGRKCVASSAAGRSRNVTVFRRCSRGGPVKVVANAGGEAGPGFLQRLGRVIKEKAAGDFDRFFAGTSKTRERLGLVDEMLALWSLEDYEDSLEELEEVLISADFGPRTALKIVDRIREGVKAGRVKSAEDIRASLKAAIVELLTARGRSSELKLQGRPAVVLIVGVNGAGKTTTVGKIAYKYGKEGAKVFLIPGDTFRAAAAEQLAEWSRRAGATIGAFREGARPQAVIASNLDDLRQRTCKDASDVYDLILVDTAGRLHTAYKLMEELALCKAAVSNALPGQPDETLLVLDGTTGLNMLNQAKEFNEAVRLSGLILTKLDGTARGGAVVSVVDQLGLPVKFIGVGETAEDLQPFDPEAFAEALFPKVKEPATAGTK

SEQ ID NO: 3 - DNA, 9870 bp, Chlamydomonas reinhardtii
  Signal Recognition Particle (SRP) component Tla2, CpFTSY genomic DNA sequence
gtgacaggcccggcgcgcacgccgcatacacaacgcgtctgcctctggccggccggcctgccgtgtctgccgctgatatgccctggtacccccgctaacccaggagcgcaagctcacccgccgatgtagaagtaggcatttaggaatagccatgcgtagaagatgacggtgcgggcccgcagcggcagcagcggccgccaagcggatgtgcgccaggacggacgcgtcgctgcgtacagcaccccgacgcccagcagcagcagcacattgatggccgctgccagaggaattgtctccgcccatggccatggaagcccccaaacgtggctttcatcgggggacagccagaccaccgctgtgccgaggatggcgaccgcgaggagcgccaggcaggggcgccaccccaccggaacttgaatgcgagccgagtaggtcatgcagctgcgcgtgctggtctccaggaggcgccgagcatgccggtagactatcctccgctttgaatcaaggtgcaatttcaggtcctcaggctgagcaacagttgcctgactcggtggagctgcaggttgtaatgggtattgtccacctcttcttcttgccgagggcacccatggcccctgcgaggccatggtttgcggcttcgcgaacacctaacttgcgtgcataaagcgtgcgacaggcacgcgccgctgctgagctggcttaatttctacagtagtctcacttgtgcaactgaatatgtaattgcgattaaggagcgaggtttgtgggctgttgcgccaatggtcactccggtacaactccggaaccgacacgcagcagtagcccccaagatttagccccaacttcagccctcaagttcagcgggtttgggtcagaacaacgacagggtgtgctgatagcgacttaacactctaagcagcaagagctggcaaggcggctttcataagcaacagccagggcaagcgtgtgctcgcgcttcagtcaaactgtcaaacacctcgtgttgttacggtatcaaggcagaagcgctctgcaaaacgactaggcctaagtggacgtgcgttctttaaatggtagcaacatggcgctgcatggcgccctgccgtttgcccgtttcccgtcgtttcccagggttggggcacccctgccgtatcccactttctcccgctctcaagcgatcagcccggtccccccttccacactgccgcgagcacacaggagaatacctggcagcatatacttgcgcaggacactgggactgcgccaatgcacgaatgtgacctgctgcggaggaacccctgtgtcgagtcgactgtttcaatgccaaacccttgagttatggtccaaggtcagacagatggtatgaaagacggtactaacacacggcagtctacgcacaatatacctcgttcggacaccgatgacctgcgatgtcgatgcgctttaagtgttccttgcatagtgtcaaaggacgggtgcggtgcgcatgaagaacctatagtgacgtccgggggtgtcaggacggggcgttttgtaaagtgaggtccagaggagcgcagggtagggggcatgtggaaggtcggggcgtgtcggggggtggggagtgggggcacgagtgggggtggggaaccgcattggagatccaggtcggttgccccggcgagttttgccgcgcgccacccaaatgcggactttaacgcttgaggaacacatacaaacagtagcgagttcgcgcctgctttccgcgtctgcaccgcactccacgtcgactgctttttcgttttcaggctttgttttacaagccgacttatcttacaattaattggttgttcatagaccctacattgatccgaggatctagcgattgtgcacaggaaagccgaaatgcagacgaccgtggggcgcaagtgcgtcgcgagctccgcagctgggcgcagccgcaatgttactgtcttccgtaggtgcagcaggggcggccctgtgaaggtacgcgacaggctcagtggccgtttggcgattgcacactcctgttcgtcgcgccagcctgcgaacccagctgtcatacttatatgatagtgtggaaggccacggtggttgggttggccagcggggctcacgcggagcggttccgcggacacggtccctgcttcagacagccctaccccttcccgccctctcatgccccacgcgcgctgctcgcggctgtgcaggtggtcgccaacgcgggcggcgaggccggccccggcttcctgcagcgcctaggccgcgtcatcaaggagaaggccgccggcgattttgaccgcttcttcgcggggacctccaagacgcgcgagcgcctgggggtaagtggcgccgtgcagtgcccttcacaagcccgcataggccgcaacacctgccgcccgacgtgctttgctgcacccgtacttgccgacccaatcaccacaaatgcacacaagcccgctccccgtgtgctttcccaacaaccaacttgcccgcagctggtggatgagatgctggcgttgtggagcctggaggactacgaggacagcctggaggagctggaggaggtgctcatcgtgagtggggcttgggcttgggcggggggtggaaggggaggggttacattcatgactagtgactagcggaaggggggttactttactccaaaggcagtgggctggtggggatggggagcgggcgggtggtcgtggtggttgcggtcgtggtgctgatcgtggatgaaggttggggcagggagggtgtgtgtgtgctggcagcagtgtgcgtgggtgtgggtgtgggtgtgggtgtgggactgtgggtgtgggtgtgggtgtgggtgtgggtgtgggtgtgggtgtgggtgtgggtgtgggtgtaggactgtgggtgtgggtgtgggtgtgggtgtgggtgtgggactgtgggtgtgggtgtgggtgtgggtgtgggtgcggtgcgggtgtgggtttgggtttgggcgtgggtgtgggtgtgggggttaactctgaaccaatcccataaggaaacagaatcgctgcacggggaagggtgtgaaggaagagggagagggtgccgtggaagacaggcacaatggatgttaggtgttgccgggggagtgccagtttgggagcggctttggatggggttgttttcaccattcccactaagaaacccacagacacgacggggggaacctagttccaaaagggcatggtcgttgcacacaagaacgcatgacatgggcagtcaagcccaaccctgaatacaggggggcagcggagcagggggcagcgcgtgctagacctgacgggtactggggtctgaacggtcggaggggacagggttcggcaagggcatgcaccgagtcccaagcgccaagggcacgcaccttgcaccgcagacccagacagccgtgtgacgggctgacggcgagaggctggctttcttggcatgacctgcatgctgcagagggatcaaggcccaagggccccgggggtgtccccgcaatcaccaacccgcccacccgcccaccccttcacccgctctcctgcgtcacctcgaatcacacacccccaccgccatccgcttctgtccacagtccgccgacttcggcccccgcactgctctgaagatcgtggaccgcatccgcgagggcgtcaaggccggccgcgtcaagagcgccgaggacatccgcgcctcgctcaaggtgggtgtgggtgtgttagtgtttgtgtttgtgtgtgtatgtgtgtgtgtgtgtgtgtgtgtgtgtgtgtgtgtgtgtgtgtgtgtgtgtgtgtgtgtgtgtgtgtcaagtacgaaacgaaaacccgcccaataacagcaccggagttaaagtgtgtgtgggggggggaggtttaacactgaaccaatcccgtaaggaaacagtcgctacaaggagaaactgcatccgcatatgcgagctacttgacttcggttgtcggggttgacttcggttgtcggggttgacttcggttgtcggggtccgcgggtcgtttactccaatgcctaagatacggggttgacatgcccgagcccattagccaaggaatcgtcgcaagggattggggggttgcagatctctaaatgaagcaggggggtgcagacggtgttgggagcacgcgtgtgcgcgtgcgtgtgcgtatctggtgcgcgtggctgggcgggtggtgtgcgtggggagggtggttcaatgtgtgcagacacgccggtccagctcagttacgcggccgtccttcacctcgctgccatggtctgtacacgcaggctgccattgtggagctgctgacggcacgggggcgctccagcgagctcaagctgcaggtgcgtcatgagggaggcggaccggaactggagaggaagccagaaacaaactgaggttcaggaaggagtgagatgacttggaccgcattcacggctgctgttaacagctgctgcagcggagcccacgcgcgagcgagcggagctcctcaaaagctaggtccctgaattgcgccgcacgttgcaacactgcagggccggccggctgtggtgctgattgtgggagtgaacggcgcgggcaagaccaccactgtgggcaagatcgcgtacaagtacggcaaggagggagccaaggtaagcttgggctggagcgggcggagcagaagaggtggaggaggagggaggaggagtagctcggaaggagggttgggtagggccggaggaggctgtggggcgactgggtgggtgcggcaggcagtggtaaagggagctgctaggcaggtggttgtgggacccgtgtcgggatgctgtacgggcgtgatggtggggagttcggtgggcagttcggatgtatcgtaagcgcgtgggcacaccagcggttgtcgaggcagcagcagctgcggcatcacgtgaccattgctacaaccctgtcagcacacgcgtccgccacgcgaatgctatatccttacatgcatacagaccgcgcgcacactcgcctccagactgctgccgttccgaaccggcatacggtatgtcgttatcctgaccgcgcgaccttgcgggcacacgcgaccgcacgtgcgctgcgcaggtcttcctcatcccaggcgacaccttccgcgccgccgccgcggagcagctggcggagtggagccgccgcgcgggcgccaccatcggcgccttccgagagggtgcccgaccgcaggccgtcatcgcctcggtgagtgcggggtgcagtgggcgtaaggggttgtgtttaccagcccaaacttgagcaagacaaagggaattcagcggagcacaggggagggggctgtgggcacggtatggggcatgttgcgggtgcggttagcggagggtgtgtgctaggggagcacgtgcagcggtgtgcagtggggcaatttcgaatcgtatagcatccctgtccttgctggtatgcgtcttgcctctgccgtgcccctcatctgtatgaccattacccactccacctttcacggacggctacccccaaccaccaccactatccgtcatcgcgcccatcggcaaccaccgtacaccaactaccaccggcccaaccaccatctccccaaccgccaaccaacagaacctggacgacctgcggcagcgcacgtgcaaggacgcgtcggacgtgtacgacctcattctggtagacacggcaggtgcgtgtctgggggcgagggttgcgggtagcgtgcaataggggttgctctaacgttcgctttccttccgcctaaaggcttgcgtccgccttcacacatcaaacgccactatccatttcccaccagcaccgccccaccaccccaaccaccgccccaaccaccgccccaaccaccgccccgaccaccaccccaaccaccgccccaaccaccgccccaaccaccacaccaccccaaccaccaggccggctgcacacggcctacaagctgatggaggagctggcgctgtgcaaggcggcggtcagcaacgcactgccgggccaggtgcgggcggcggacggggggttacattcatgatatttaagaagtgaagtcgtagggggggcgggcgttgacgttggcgctgttgcgtctttgcgtggatgcgtggcggggcgccggtgctcctcaagccggatctgcagtgcctgaaggcctcgcgctgccaggctgccatgcatgcggtccatgcgacccttgcctgggccgtctgccgcaaccagccccgatcacagtccgccccacgcatctatctgtgcacgccgttaactcatcaattggcaatgcctggtgatgcccgctccttgttgccgccccgcgcagcccgacgagaccctacttgtgctagacggcaccacaggcctcaacatgctaaaccaggtggggcggagtgcaggatgtgtgtatgtgttatacatgcaaaggtgcaaaacgcaaaggcacagtcggcgggtggtgccgcgccggccccactttgcgccaccgcatcatatgcgctacccggcgcctttctgcctggagtcctgagctctagctgcccaacaggcccttcccttgtgctctctaacctcgcccaaccgctgcgcttgcaggccaaggagttcaacgaggctgtgcggctgagcggactcatcctcacaaagctggatggcactgcccgaggaggtgcgcatatgtgtgtgtgtgtgtctgtgcgtgtctgtgcgtgtaccgtatgtgtatgcgtgtgtgcgtgtgcgtgtgcttgtagtcccacgcgtatcaatcgcccgcccccggcttgcaaggatacctctcatgactcatggggcactgtaaatgcaactggtgcatagggaagcacaagggaaaagggtcgataccatttaaatgcgtggggttgcggcgttgtattcacactgccccaagcattcctgacaacggcaggcagtgaccagtggatttgtgagtgtgaggcgcccattgctgcacctcccggtgttgctccatagcccaccgttctgtgtccgccggtccttagtgtgtctaccgcttgatttctccttgccattcgcttggtttggttagtttaggtttgggctggtacccaagctacccctgcccccctgctgttccgtgtctctcaccccgtgtcgctgtccccctccaggcgcggtggtgagtgtggtggaccagttgggcctgcccgtcaagttcattggtgtgggcgagacggccgaggacctgcagcccttcgaccccgaggtgggcagcagcagaccgggggggcggggggtgggggggccagccgtggctcgaatgggggagaagcggagagtgggggaggcggcacggggaggcggtggggagggttacggggatcaaacacataccgtaggagcgctgggctgcactgcaatgcccctgcgtccccgcgggggcgaggcttacggcagttgggttgcagtcatgaacggggcattctgcgtgcctggtttgcagtagcgagcggggggtgacggcgttggcgttggcgtgcgcaaatgcacacacgctttgcaaattccacaccaccgctctgcacgtccggaacgacgcagcgtccggcgccccgctccatccccccctttggggttggaacaaactactccctaaaatacaagtacaatgccgccttgccccttctatccccccgacgcacccagccgactttccctatccgatgccggtgcctgtcctgcctgtcgctacacaggcattcgctgaggccctgttcccgaaggtcaaggagcccgccactgccggcaccaagtaaatgaacggggcggagcactgctgggggcgcctgggggcaaatacgtttgggcatgctggtatggatgtgggcttacttgttttctaagtggaggtgggctttgggatgcaggtgtgtgaagtctgtactgcgtttcgtgttggtttgtgcgagagagcgcgatgagtgactcgtggtgtgtcgccatttgcaaggaatttcaaacctgcgtgagggaaggcaacaggggttgcggtcactgggtgcgcccctgcaaggccaaagttgataggatggcgcgattggcgcctaaagcgtttatactgctcgcggcacagaactgggcgggtacgccttcgcaatgtatggaggtgcagtttgtggatgccggtctctttggcgcgccgcactcttgtggaagtgggtcgccgcggtgtggcgaacggcctaaggcggcaacgggcaacgcgtgtgaacgagggtgtgcgacgggacaacggtggcaaggcaggttgggaagtcccgtaggatgcagctgattcccagaacccagcagatggcaaacaatttgcaatgggcaacgcgtgtgaacgaggggagcacggagcaccctggcgggcacccggcacgccagctgtgcaagcagtggtactggcctaggtggacgagggcggcctgcccttgtcggccactcagcccggtacttgcagtggaagcggaggagggatgcgagcggccacctaggcaatcagcgtggcggcggactagcggcggcctcctgggggcggaggagtgcaaaggaacgtgtaaatacggaggaagcggagggaaggaagcaataggaggtgtggctgtcgtaggctcggatggaatggtggagcaaggcagagcacgtaaggcggagcatgactgggacaagcaggcagcacagccgttgtgcaagcactccgtcttcatttgtgctgtaagggatgataaggtgcatatgaggtttctggcccccggcgcggtgggtgtcgtgtggcaagtggcaagggagcttcgcgtgttgggacgtgaagtccggtgggggcgcgagcccatgtgcccatccagcgaagccctagcgcctgcgtctgcgcgcatgtgaaggcgggggcaagcgctgtcagagcgccaggtggatgtgggccttgtgggcggaacagcggggggcggcaggtgtaggtggcgagggcatcggctcaagagcctcgggagtgcctcgggggcccgggggcccaagcggcagcatgatgcgagtggcgcacgcattgtgtgcattccgcatccggtcaagcgcggcacattccatgctgttgtgctttcactacttgcgcaactgctacatgccgacacacaaatctgtactcggtgcagttcactgtggcttggctcccattcgctttgaccgcaataacctactttgccgccgagcgagcggctggcagcgagtgtttgataccttgcgcacctctaagtgttatttaggcgatagcaagcttcagtcacgtcactgcacgtgctcatttctcggttcttgggagtgcatgccctggtttcggccccgactagtgccgcgcgcccctgcgtgtgagcacaaatacctttgcccagcatatcatatgtttgctatgaacggcagcagtctacatcgacgactgtcggagcgtgtcggagcgtgaacccggctgcacgccgacgtggctcgcttcaatccttgggggacgccctgcgcccttcacctgcccacgtaagctttgctgcgcgagtcatgcaatagcacataatgagtagtgccattgcattgctgtagtaccgtgcgagtcgggctgcaagcggttgggcgcgtacctacccgtgtatggctgcggccgcgctaaatgtgccccgtgggtgggcgagctctatatcgcgctgcacgggttgacagcacagcaatagattggccgcccagcttgcatagctcgatggtgtacgcgccgggcggcacgttgtcaagcgccgccaggatggagggcggcagcttcggctggctgctgaatcgctgaatctcctccagcgcgtcagagtccagct

SEQ ID NO: 4 - DNA, 20 bp, Artificial Sequence
  synthetic PCR primer K001
attgggcgctcttccgcttc

SEQ ID NO: 5 - DNA, 21 bp, Artificial Sequence
  synthetic PCR primer K002
gcctcactgattaagcattgg

SEQ ID NO: 6 - DNA, 23 bp, Artificial Sequence
  synthetic PCR primer K003
tatgagtaaacttggtctgacag

SEQ ID NO: 7 - DNA, 22 bp, Artificial Sequence
  synthetic PCR primer K004
aggaagagtatgagtattcaac

SEQ ID NO: 8 - DNA, 25 bp, Artificial Sequence
  synthetic PCR primer K081
ggaataagggcgacacggaaatgtt

SEQ ID NO: 9 - DNA, 27 bp, Artificial Sequence
  synthetic PCR primer K082
ctcctttcgctttcttcccttcctttc

SEQ ID NO: 10 - DNA, 24 bp, Artificial Sequence
  synthetic PCR primer K094
ctagaactagtggatcccccgaac

SEQ ID NO: 11 - DNA, 21 bp, Artificial Sequence
  synthetic PCR primer K095
ctcatcctcctcgcactcgtg

SEQ ID NO: 12 - DNA, 21 bp, Artificial Sequence
  synthetic PCR primer K096
gttacaagcgacgaatgcgtg

SEQ ID NO: 13 - DNA, 20 bp, Artificial Sequence
  synthetic PCR primer K097
ctgtgccgcaccttgatgtc

SEQ ID NO: 14 - DNA, 22 bp, Artificial Sequence
  synthetic PCR primer K038
gtttgtgcaggagtgttgggag

SEQ ID NO: 15 - DNA, 22 bp, Artificial Sequence
  synthetic PCR primer K039
aacgttcgatagctctcacaac

SEQ ID NO: 16 - DNA, 26 bp, Artificial Sequence
  synthetic PCR primer K120
ccactacgtgaaccatcaccctaatc

SEQ ID NO: 17 - DNA, 25 bp, Artificial Sequence
  synthetic PCR primer K119
agaccgagatagggttgagtgttgt

SEQ ID NO: 18 - DNA, 20 bp, Artificial Sequence
  synthetic PCR primer K118
caaataggggttccgcgcac

SEQ ID NO: 19 - DNA, 25 bp, Artificial Sequence
  synthetic PCR primer K081
ggaataagggcgacacggaaatgtt

SEQ ID NO: 20 - DNA, 27 bp, Artificial Sequence
  synthetic PCR primer K082
ctcctttcgctttcttcccttcctttc

SEQ ID NO: 21 - DNA, 14 bp, Artificial Sequence
  synthetic PCR primer K030
ntcgwgwtscnagc

SEQ ID NO: 22 - DNA, 14 bp, Artificial Sequence
  synthetic PCR primer K031
wgntcwgncangcg

SEQ ID NO: 23 - DNA, 30 bp, Artificial Sequence
  synthetic PCR primer K121
gacggtttttcgccctttgacgttggagtc

SEQ ID NO: 24 - DNA, 26 bp, Artificial Sequence
  synthetic PCR primer K122
cacgttctttaatagtggactcttgt

SEQ ID NO: 25 - DNA, 18 bp, Artificial Sequence
  synthetic PCR primer K123
ccaaactggaacaacact

SEQ ID NO: 26 - DNA, 40 bp, Artificial Sequence
  synthetic PCR primer K124
aacgcgaattttaacaaaatattaacgcttacaatttagg

SEQ ID NO: 27 - DNA, 21 bp, Artificial Sequence
  synthetic PCR primer K125
tggcacttttcggggaaatgt

SEQ ID NO: 28 - DNA, 24 bp, Artificial Sequence
  synthetic PCR primer K126
ggaacccctatttgtttatttttc

SEQ ID NO: 29 - DNA, 24 bp, Artificial Sequence
  synthetic PCR primer K169
gtccaacatgggcgaccgcatctg

SEQ ID NO: 30 - DNA, 21 bp, Artificial Sequence
  synthetic PCR primer K170
catgccggacccgttaacatc

SEQ ID NO: 31 - DNA, 19 bp, Artificial Sequence
  synthetic PCR primer K171
ctccaccaacgtcaccatc

SEQ ID NO: 32 - DNA, 34 bp, Artificial Sequence
  synthetic PCR primer K214
gtttatcagattgagagtgaaactggcgctttac

SEQ ID NO: 33 - DNA, 23 bp, Artificial Sequence
  synthetic PCR primer K215
catgacctactcggctcgcattc

SEQ ID NO: 34 - DNA, 26 bp, Artificial Sequence
  synthetic PCR primer K131
caaaacctcaccgtggatttcgtcaa

SEQ ID NO: 35 - DNA, 28 bp, Artificial Sequence
  synthetic PCR primer K132
gggttgtaataccgtggggattttaagc

SEQ ID NO: 36 - DNA, 30 bp, Artificial Sequence
  synthetic PCR primer K172
gtagactatcctccgctttgaatcaaggtg

SEQ ID NO: 37 - DNA, 28 bp, Artificial Sequence
  synthetic PCR primer K173
cacgaggtgtttgacagtttgactgaag

SEQ ID NO: 38 - DNA, 19 bp, Artificial Sequence
  synthetic PCR primer K193
gcttacgtggagcgtgcag

SEQ ID NO: 39 - DNA, 24 bp, Artificial Sequence
  synthetic PCR primer K194
cagtttactggtgtcgtggtgcac

SEQ ID NO: 40 - DNA, 21 bp, Artificial Sequence
  synthetic PCR primer K216
cacccaacctcacgaccacac

SEQ ID NO: 41 - DNA, 31 bp, Artificial Sequence
  synthetic PCR primer K217
cacccctccgtgttgtaacctatacaactac

SEQ ID NO: 42 - DNA, 29 bp, Artificial Sequence
  synthetic PCR primer K220
gtggcacttttagtgtcaagcatactgtg

SEQ ID NO: 43 - DNA, 23 bp, Artificial Sequence
  synthetic PCR primer K221
ggatgcggaatccacccacaatg

SEQ ID NO: 44 - DNA, 29 bp, Artificial Sequence
  synthetic PCR primer K220
gtggcacttttagtgtcaagcatactgtg

SEQ ID NO: 45 - DNA, 22 bp, Artificial Sequence
  synthetic PCR primer K238
cctgaacaagggtgcatgcatg

SEQ ID NO: 46 - DNA, 19 bp, Artificial Sequence
  synthetic PCR primer K237
gtcgtgcgcgtttcaggtc

SEQ ID NO: 47 - DNA, 37 bp, Artificial Sequence
  synthetic PCR primer K236
cttcggaaccattgttgattacacttctataaagcag

SEQ ID NO: 48 - DNA, 37 bp, Artificial Sequence
  synthetic PCR primer K240
caccttatcattccttacagcacaaataaagactgag

SEQ ID NO: 49 - DNA, 25 bp, Artificial Sequence
  synthetic PCR primer K128
gctttctaccgcctgtctcaatacc

SEQ ID NO: 50 - DNA, 24 bp, Artificial Sequence
  synthetic PCR primer K126
ggaacccctatttgtttatttttc

SEQ ID NO: 51 - DNA, 25 bp, Artificial Sequence
  synthetic PCR primer K134
gcagccttgccaatcccaaataagg

SEQ ID NO: 52 - DNA, 26 bp, Artificial Sequence
  synthetic PCR primer K135
ctccgcacattcacattaggaggttc

SEQ ID NO: 53 - DNA, 23 bp, Artificial Sequence
  synthetic PCR primer K289
gatgcggtccacgatcttcagag

SEQ ID NO: 54 - DNA, 23 bp, Artificial Sequence
  synthetic PCR primer K290
gagatgagcacctcctccagctc

SEQ ID NO: 55 - DNA, 26 bp, Artificial Sequence
  synthetic PCR primer K297
ctgtggtgctgattgtgggagtgaac

SEQ ID NO: 56 - DNA, 25 bp, Artificial Sequence
  synthetic PCR primer K298
caagatcgcgtacaagtacggcaag

SEQ ID NO: 57 - DNA, 36 bp, Artificial Sequence
  synthetic PCR primer K345
ggaattccatatgcagacgaccgtggggcgcaagtg

SEQ ID NO: 58 - DNA, 34 bp, Artificial Sequence
  synthetic PCR primer K346
ggaattccgttcatttacttggtgccggcagtgg

SEQ ID NO: 59 - DNA, 29 bp, Artificial Sequence
  synthetic PCR primer ET28-ftsY_up
gggaattccatatggccaacgcgggcggc

SEQ ID NO: 60 - DNA, 30 bp, Artificial Sequence
  synthetic PCR primer ET28-ftsY_down
ccggaattcttacttggtgccggcagtggc

SEQ ID NO: 61 - DNA, 35 bp, Artificial Sequence
  synthetic PCR primer ET28-srp54_up
gggaattccatatgtcggccatgttcgacagcctg

SEQ ID NO: 62 - DNA, 34 bp, Artificial Sequence
  synthetic PCR primer ET28-srp54_down
ccggaattcctatttggacgatgagccgaagccg